(19) United States
(12) Patent Application Publication
(10) Pub. No.: US 2017/0226503 A1
(43) Pub. Date: Aug. 10, 2017

(54) Methods for Sequential Screening with Co-Culture Based Detection of Metagenomic Elements Conferring Heterologous Metabolite Secretion

(71) Applicant: MetaMixis Biologics, Inc., Vancouver (CA)

(72) Inventors: Cameron R. Strachan, Menlo Park, CA (US); Steven Hallam, Vancouver (CA)

(73) Assignee: MetaMixis Biologics, Inc., Vancouver, BC (CA)

(21) Appl. No.: 15/115,644

(22) PCT Filed: Feb. 6, 2015
(86) PCT No.: PCT/CA2015/000071

§ 371 Date: Jul. 29, 2016

Related U.S. Application Data
(60) Provisional application No. 61/936,448, filed on Feb. 6, 2014.

(51) Int. Cl. C12N 15/10

(52) U.S. Cl. CPC C12N 15/1079; C12N 15/1082; C12N 15/1086

(57) ABSTRACT

The present invention relates to methods associated with metagenomic screening for metabolite induced elements (MIEs) and the subsequent use of the MIEs in screening metagenomic libraries to identify metabolic pathways and pathway components in one or more partial or complete operons. In one aspect the method may be an iterative approach to metagenomic screening which involves substrate and product selection.

### TECHNICAL FIELD

This invention relates to the field of metagenomic screening. In particular, the invention relates to functional metagenomic library screening methods for detecting metabolite secretion or extracellular chemical transformations.

### BACKGROUND

It has long been appreciated that environmental micro-organisms are an excellent source of solutions to industrial problems. In particular, they may provide a source for enzymes and associated co-factors. However, there is also an increasing awareness that environmental microorganisms can be difficult to culture in the laboratory let alone on an industrial scale.

For example, lignin is the second most abundant biopolymer on earth and a promising feedstock for deriving energy and industrial chemical precursors from renewable plant resources^{6,7}. The synthesis of lignin occurs within plant cell walls by free radical reactions that cross-link diverse combinations of monoaromatic compounds into a heterogeneous matrix that is resistant to microbial and chemical assailment⁸. Lignin recalcitrance is further reflected in the deposition of coal throughout the Carboniferous period prior to the emergence of fungal enzymes associated with lignolysis in Permian forest soil ecosystems⁹. Although a few bacterial strains and enzymes capable of lignin transformation have been identified, including Enterobacter lignolyticus SCF1 and Rhodococcus jostii RHA1¹⁰⁻¹², white-rot basidiomycetes are currently the major source of lignin transforming enzymes, including laccases, manganese-dependent peroxidases, and lignin peroxidases¹³. This presents numerous technical challenges associated with the genetic tractability of fungal systems and the expression of fungal-derived enzymes in heterologous hosts such as E. coli¹⁴. Implementing high-throughput methods to expedite the discovery of bacterial lignin transformation pathway components provides one promising route toward overcoming these challenges. However, to date efforts to develop such functional screens have been unreliable due to the inherent complexity of the lignin polymer¹⁵.

A number of metagenome screening methods have been developed to isolate useful genes from metagenomes. For example, metagenomic nucleotide sequencing methods¹, and enzyme activity based screening². Further enzyme activity based screening methods have been developed, such as Substrate-Induced Gene-Expression (SIGEX) screening³ and more recently Product-Induced Gene-Expression (PIGEX) screening⁴. Furthermore, several screening strategies have been developed to discover genetic elements that are activated in response to a metabolite, including intragenic genomic libraries and promoter traps⁵.

### SUMMARY

The present application is based in part on the discovery that previously uncharacterized pathways or unknown enzymes or cofactors in a pathway may be identified using the methods described herein. Furthermore, based on insights gained herein, it has been discovered that the process may be applied in an interative manner to discover metabolite inducible elements (MIE) of interest under inducible expression control.

In most known metagenomic screening processes there is often a shortage of MIEs and inherent host incompatibility associated with the MIEs. These may in part be alleviated by screening environmental DNA to identify new MIEs from the same or similar functional metagenomic libraries. Such an approach also makes the processes described herein iterative and agile. Whereby pools of metabolite compounds may be used to screen for MIE's, where different metabolites of interest may be identified or different intermediates in a pathway may be screened to find MIEs. Accordingly, where a step or steps in a pathway was missing or where there was a desire to expand the biosynthesis pathways the method could easily be repeated with a different metabolite of interest and could perhaps even include the addition of further clones in co-culture. Furthermore, it was appreciated herein that the use of intermediate to large insert metagenomic libraries (5-45 KB) is beneficial to the success of the methods. For example, where several genes are present in an operon, it is sometime possible to clone the entire operon of interest with an intermediate to large insert library in association with MIEs and their cognate transcriptional regulators. Furthermore, the use of vectors able to accommodate large inserts (for example, fosmids) can be helpful. Furthermore, a transposon retrofitted MIE library has advantages to other MIE screening methods such as restriction digestion libraries. Restriction digestion libraries have several limitations. For example, if any regulators or machinery is found downstream (for example, beyond an operon and is necessary for the MIE) such other methods would miss it, since the reporter in these systems is the last gene in the construct and thus could inherently limit what could be retrieved.

There are biases based on transcription in any given bacterial host (for example, E. coli), but there is actually a lot of conservation with respect to transcription and translation control across taxa. In fact, some components of the transcription and translation machinery are so conserved they may be used as phylogentic anchors to differentiate taxa on the tree of life.

Often, in functional metagenomic screens, the metabolite of interest is only one enzymatic conversion away from the substrate. Focusing on degree of separation away greatly limits the ability to recover more extensive biosynthetic pathways, whether they comprise an operon, interact with host metabolism, or act in a segmented or distributed pathway between two or more members of the community. This is because the substrate selection creates a bias against the preceding steps in the biosynthesis pathway. Accordingly, to be sure that the biosynthetic pathway of interest is selected, it is often important to consider the media (for example, are all substrates present) and the final product you are interested in detecting.

In accordance with a first aspect of the invention, there is provided a method including: (a) randomly inserting a mobile genetic element into a first metagenomic library to produce a randomly inserted first metagenomic library, wherein the mobile genetic element comprises a promoter-less reporter gene and selectable marker; (b) screening the randomly inserted first metagenomic library by adding a metabolite of interest; (c) detecting reporter gene expression following the addition of the metabolite of interest to identify a metabolite induced element (MIE); (d) preparing a reporter strain, the reporter strain including: (i) the MIE; and (ii) a reporter gene adjacent the MIE; (e) co-culturing heterologous host cells expressing a second metagenomic library with the reporter strain; and (f) detecting the reporter gene activity in the co-culture.

In accordance with another aspect of the invention, there is provided a method including: (a) obtaining a reporter strain, the reporter strain including: (i) a metabolite induced element (MIE), wherein the MIE is responsive to a metabolite of interest; and (ii) a reporter gene adjacent the MIE; (b) co-culturing heterologous host cells expressing a functional metagenomic library with the reporter strain; and (c) detecting the reporter gene activity in the co-culture.

In accordance with another aspect of the invention, there is provided a method including: (a) obtaining a reporter construct, the reporter construct including: (i) a metabolite induced element (MIE), wherein the MIE may be responsive to a metabolite of interest; and (ii) a reporter gene; (b) transforming a reporter strain with the reporter construct from (a); (c) co-culturing the reporter strain with a heterologous host cells expressing a functional metagenomic library; and (d) detecting the reporter gene activity in the co-culture.

In accordance with another aspect of the invention, there is provided a method including: (a) obtaining a reporter construct, the reporter construct including: (i) a metabolite induced element (MIE), wherein the MIE may be responsive to a metabolite of interest; and (ii) a reporter gene; (b) transforming a cell with the reporter construct from (a) to form a reporter strain; (c) growing heterologous host cells expressing a functional metagenomic library; (e) adding the reporter strain from (b) to the heterologous host cells expressing a functional metagenomic library to form a co-culture; and (f) detecting the reporter gene activity in the co-culture.

The method may further include testing the MIE for specificity to the metabolite of interest prior to co-culturing the heterologous host cells expressing a functional metagenomic library with the reporter strain. The method may further include testing the MIE for sensitivity to the metabolite of interest prior to co-culturing the heterologous host cells expressing a functional metagenomic library with the reporter strain. The method may further include testing the MIE for avidity to the metabolite of interest prior to co-culturing the heterologous host cells expressing a functional metagenomic library with the reporter strain.

The method may further include engineering the MIE to obtain the desired substrate specificity, sensitivity, and/or avidity following testing the MIE for specificity, sensitivity and/or avidity to the metabolite of interest.

The functional metagenomic library may be a fosmid library. The method may further include mutagenesis of functional metagenomic host cells producing a product that results inreporter strain activity. The method may further include screening for production of the metabolite of interest.

The reporter strain cells and the heterologous host cells expressing a functional metagenomic library may be cultured in a plate-based format. The MIE may be obtained from a functional metagenomic library.

The reporter strain may be a bacterial cell. The heterologous host cells expressing a functional metagenomic library may be bacterial cells. The bacterial cell may be an E. coli cell.

The method may further include isolating the co-culture having reporter gene activity.

The method may further include culturing the host cells having reporter gene activity to produce the metabolite of interest.

In accordance with another aspect of the invention, there is provided a method including: (a) choosing a first metabolite of interest and a first substrate; (b) randomly inserting a mobile genetic element into a first metagenomic library to produce a randomly inserted first metagenomic library, wherein the mobile genetic element comprises a promoter-less reporter gene; (c) screening the randomly inserted first metagenomic library by adding the first metabolite of interest; (d) detecting reporter gene expression following the addition of the first metabolite of interest to identify a first metabolite induced element (MIE1); (e) preparing a first reporter strain, the reporter strain including: (i) the MIE1; and (ii) a reporter gene adjacent to MIE1; (f) co-culturing heterologous host cells expressing a second metagenomic library with the first reporter strain in the presence of the first substrate; (g) detecting the reporter gene activity in the co-culture; and (h) repeat steps (a)-(f) as desired, wherein the first metabolite of interest may be used as a second substrate and a new metabolite of interest may be a second metabolite of interest and may be used to generate an MIE2.

The method may further include testing the one or more MIEs for specificity to the metabolites of interest prior to co-culturing the heterologous host cells expressing a functional metagenomic library with the reporter strains. The method may further include testing the one or more MIEs for sensitivity to the metabolites of interest prior to co-culturing the heterologous host cells expressing a functional metagenomic library with the reporter strains. The method may further include testing the one or more MIEs for avidity to the metabolites and DNA binding site of interest prior to co-culturing the heterologous host cells expressing a functional metagenomic library with the reporter strains.

The method may further include engineering the one or more MIEs to obtain the desired substrate specificity, sensitivity and/or avidity following testing the one or more MIEs for specificity, sensitivity and/or avidity to the metabolites of interest.

The functional metagenomic library may be a fosmid library.

The method may further include mutagenesis of functional metagenomic host cells producing reporter strain activity and further screening for production of the metabolite of interest.

The reporter strain cells and the heterologous host cells expressing a functional metagenomic library may be cultured in a plate-based format.

The one or more MIEs may be obtained from a functional metagenomic library. The reporter strain may be a bacterial cell. The heterologous host cells expressing a functional metagenomic library may be bacterial cells. The bacterial cell may be E. coli cells.

The method may further include isolating the co-culture having reporter gene activity. The method may further include culturing the host cells having reporter gene activity to produce the metabolite of interest.

In accordance with another aspect of the invention, there is provided a method including the steps of: (a) randomly inserting a mobile genetic element into a first metagenomic library to produce a randomly inserted first metagenomic library, wherein the mobile genetic element comprises a promoter-less reporter gene; (b) screening the randomly inserted first metagenomic library by adding a metabolite of interest; (c) detecting reporter gene expression following the addition of the metabolite of interest to identify a metabolite induced element (MIE); and (d) preparing a reporter strain, the reporter strain including: (i) the MIE; and (ii) a reporter gene adjacent the MIE.

The method may further include the step of: (e) co-culturing heterologous host cells expressing a second metagenomic library with the reporter strain. The method may further include the step of: (f) detecting the reporter gene activity in the co-culture. The method may further include testing the MIE for specificity and sensitivity to the metabolite of interest prior to co-culturing the heterologous host cells expressing a functional metagenomic library with the reporter strain. The method may further include engineering the MIE to obtain the desired substrate specificity and sensitivity following testing the MIE for specificity and sensitivity to the metabolite of interest. The functional metagenomic library may be a fosmid library. The method may further include mutagenesis of functional metagenomic host cells producing reporter strain activity and further screening for production of the metabolite of interest. The reporter strain cells and the heterologous host cells expressing a functional metagenomic library may be cultured in a plate-based format. The MIE may be obtained from a functional metagenomic library. The reporter strain may be a bacterial cell. The heterologous host cells expressing a functional metagenomic library may be bacterial cells. The bacterial cell may be an E. coli cell. The bacterial cells may be E. coli cells. The method may further include isolating the co-culture having reporter gene activity. The method may further include culturing the host cells having reporter gene activity to produce the metabolite of interest.

### BRIEF DESCRIPTION OF THE DRAWINGS

In drawings which illustrate embodiments of the invention:

FIG. 1 shows PemrR-GFP biosensor discovery and characterization, wherein (A) Screening E. coli intragenic regions with monaromatic lignin transformation products including vanillin, vanillic acid, p-coumaric acid, vanillyl alcohol and veratryl alcohol. (B) Relative reporter signal after incubation with 0.5 mM of select benzene derivatives for 2 hrs. Tree represents hierarchical clustering of the compound similarity using the single linkage algorithm. (C) Reporter sensitivity after 2 hrs. (D) Monitoring in vitro lignin oxidation by DypB N246A in the presence of glucose oxidase, manganese and hydrogen peroxide. Controls did not contain manganese. Error bars represent 95% confidence intervals (n=3);

FIGS. 2A and 2B show the profiling of monoaromatic compounds by GC-MS, wherein relative ratios of lignin related monoaromatic compounds in culture supernatant as compared to a control strain harboring an empty fosmid. Clones were incubated with both (A) HKL-F1 and (B) HP-L™ in minimal media;

FIG. 3 shows genetic context maps for active fosmids, wherein functional classes related to lignin degradation, CAZy auxiliary enzymes, mobile elements, transposon insertions (Z-score ratio cutoff for decrease in GFP fluorescence, see TABLE 2), and tRNAs are annotated. The G+C ratio for every 200 nucleotides and gene abundance determined by mapping over 500 million illumina reads sourced from the coal bed milieu is also displayed. Connections represent protein homologs with minimum 50% identity and an e-value of 10E-20;

FIGS. 4A and 4B show the effect of plasmid copy number on PemrR-GFP biosensor activation, wherein compound dependent activation of the PemrR-GFP biosensor was assessed after 2 hrs under single copy (left) and high-copy (right) number for concentrations of (A) 1 mM and (B) 0.25 mM (Error bars represent 95% confidence intervals (n=3));

FIG. 5 shows the screening environmental isolates with the PemrR-GFP biosensor, wherein soil isolates, including known lignin degraders R. jostii RHA1 and E. lignolyticus SCF1, were cultured in the presence of HP-L™ for 2 weeks with (left) and without (right) a solid phase of 0.4% agarose, then culture supernatant was then added to an PemrR-GFP biosensor culture and incubated for 2 hrs before measuring fluorescence (Error bars represent 95% confidence intervals (n=3));

FIGS. 6A-C show emrRAB promoter activation in emrR and emrB knockout backgrounds, wherein the time course GFP fluorescence measurements for 1 mM of vanillin (∘), vanillic acid (□), and vanillyl alcohol (Δ) in (A) wild-type, (B) emrR and (C) emrB knockout backgrounds (n=3);

FIGS. 7A-F show the growth kinetics of emrR and emrB knockouts in sub-inhibitory concentrations of monoaromatic compounds, wherein wild-type (Δ), emrR(−) (∘), and emrB(−)(□) strains were grown in the presence of 0.5 mM of various monoaromatic compounds (n=3) as follows (A) control, (B) vanillic acid, (C) ferulic acid, (D) vanillin, (E) salicylic acid and (F) 4-benzoic acid;

FIG. 8 shows the effect of emrR knockout on growth kinetics in the presence of enzyme treated lignin, wherein the effect of 0.5 g/L of HWKL F1 (∘⋄Δ) and DypB N246A treated HWKL F1 (□∇X) in emrR and emrB knockout backgrounds (n=3);

FIGS. 9A-D show EmrR overexpression improves growth kinetics in inhibitory levels of monoaromatics, wherein growth kinetics with uninduced (circle) and induced (square) expression of emrR from pBAD24 (Error bars represent 95% confidence intervals (n=3)) as follows (A) control, (B) vanillic acid, (C) 4-hydroxybenzoic acid and (D) caffeic acid;

FIGS. 10A and B show Fosmid library screening by co-culture with the PemrR-GFP biosensor, wherein (A) Screening results for 8×384-well plates with selected hits (7 diamonds above 1.10 fold increase). (B) Validation of select fosmid clones by repeat screening. Error bars represent 95% confidence intervals (n=3); and

FIG. 11 shows precipitation phenotypes, wherein various fosmid clones incubated alone or in combination with HWKL F1 or HP-L™ in minimal media for 16 hrs.

FIG. 12 shows GC-MS profiles of transposon mutants, wherein the chromatograms compare two transposon mutants identified by screening with the PemrR-GFP biosensor (both interrupting putative oxidoreductase open reading frames). The data was normalized to an empty fosmid clone and lignin related compounds 2,4-dihydroxybenzoic acid, 1,4-dihydroxy-2,6-dimethoxybenzene and benzoic acid are marked by A, B and C, respectively (n=2).

FIG. 13 shows comparative analysis of active fosmids, wherein the bar graphs show the relative number of annotated genes falling within the six functional classes implicated in lignin transformation phenotypes (out of 813 total genes).

FIG. 14 shows a graphic overview of an embodiment of the method for isolating metabolite induced elements (MIEs) from a metagenomic library and construction of a metagenomic library for sequential screening with co-culture based detection of meteagenomic elements conferring heterologous metabolite secretion products from a functional metagenomic library.

FIG. 15 shows a graphic representation of environmental DNA (i.e. metagenomic DNA) libraries being “retroffited” randomly with a promoter less reporter gene (arrows) to produce clones that are screened for induction by addition of a metabolite of interest to identify a metabolite induced element (MIE).

FIGS. 16A and B show a fluoresence plot for a retroffited metagenomic library (constructed using the method in FIG. 15) that was assayed for fluorescence emitted by a fluorescent marker wherein the library was (A) Uninduced (i.e. no metabolite of interest is added) and (B) Induced (i.e. where the metabolite of interest is added—a pool of pCoumaric acid, Vanillic acid and Vanillin), also showing a circled data point that represents a fosmid clone harboring a putative MIE (p10c20) selected for further investigation.

FIG. 17 shows a bar graph of an assay to validate the MIE identified in FIG. 16 (p10c20), wherein the MIE p10c20 was found to be most responsive to 1 mM pCoumaric acid.

### DETAILED DESCRIPTION

Various alternative embodiments and examples are described herein. These embodiments and examples are illustrative and should not be construed as limiting the scope of the invention.

### Definitions

As used herein ‘metagenomic’ is meant to include any genetic material obtained from an environmental source, as opposed to a laboratory cultured source. In many cases the actual origin (i.e. species or strain) of organism from which the genetic material is obtained may not be known.

As used herein a ‘functional metagenomic library’ is a gene library produced from a metagenomic source or sources, wherein the genes within the library are capable of expression.

As used herein ‘mobile genetic element’ is meant to include any type of nucleic acid molecule that is capable of movement within a genome and from one genome to another. For example, transposons or transposable elements (including retrotransposons, DNA transposons, and insertion sequences); plasmids; bacteriophage elements (including Mu; and group II introns.

As used herein ‘promoter’ is meant to include any regulatory region of DNA often acting as a control sequence to regulate adjacent gene transcription.

As used herein ‘reporter’ or ‘reporter gene’ are used interchangeably and are meant to include any gene that when expressed produces a detectable product (for example green fluorescent protein (gfp); luciferase; β-galactosidase (LacZ); β-glucuronidase (GUS); chloramphenicol acetyltransferase (cat); or neomycin phosphotransferase (neo) to name just a few). The detection may be based on a coloured product, a fluorescent, a resistance to an antibiotic or other chemical substrate, etc. Reporters are often placed adjacent to a regulatory sequence and may be an indicator of another genes activity or in the case of the metabolite induced element (MIE) it may be an indication of the activation of the reporter by a metabolite of interest acting through a transcriptional regulatory mechanism and thereby an indication that the metabolite of interest is present.

As used herein ‘metabolite induced element’ or ‘MIE’ refers to one or more of the following: a promoter; an enhancer; an operator region; a transcriptional regulator; a DNA aptamer; or RNA aptamer, which facilitate a change in gene expression based on the presence of a metabolite of interest.

As used herein ‘a reporter strain’ is meant to refer to a cell comprising an MIE and a reporter gene adjacent the MIE.

As used herein ‘specificity’ is meant to refer to the dynamic range of metabolites that activate a given MIE.

As used herein ‘sensitivity’ is meant to refer to the dynamic range of reporter outputs possible with a given MIE.

As used herein ‘avidity’ is meant to refer to the accumulated strength of multiple affinities of individual binding reactions.

Several screening strategies have been developed to discover genetic elements that are activated in response to a metabolite or a Metabolite Inducible Element (MIE), including promoter trap and intragenic genomic libraries. The process described herein may begin by applying one of these methods to recover an inducible element that may then be further engineered, if necessary, to obtain the desired substrate specificity and sensitivity. Alternatively, a MIE may be obtained from a MIE library already discovered.

These known MIEs may also, if necessary to obtain the desired substrate specificity and sensitivity, be further engineered. The MIE may then be placed adjacent (usually upstream) of a marker gene, (for example, green fluorescent protein (GFP)), and transformed into a bacterial strain (for example, E. coli) to generate a reporter strain. Furthermore, it is also possible that a reporter strain may be developed to identify multiple metabolites of interest, by placing different MIEs adjacent different reporters (for example, a product that fluoresces in a different colour). Such reporters may be all in a single reporter strain. Target metabolites may then be selected based on the potential benefit to industry (for example, secreted and/or synthesized by a fermentative organism). Examples include, valuable isomeric compounds used as intermediates in the production of pharmaceuticals and those that can replace expensive crude oil dependent synthesis. The reporter strain therefore senses the presence of a valuable compound/metabolite input and generates an output that can be easily measured with spectroscopic robotics. Functional metagenomic libraries may be constructed in heterologous hosts, (for example, E. coli), to bioprospect the metabolic potential of uncultivated microbes from natural and human engineered ecosystems. Common vehicles for this process are fosmids as they have copy-control systems available for modulating gene expression and can stably harbor up to 40 kB of environmental DNA. The ability to harbor over 40 kB of environmental DNA is important since microbial genes are often found in operons, whereby the genes contained therein are regulated by a single promoter or regulatory signal, and work together to achieve a particular goal. For example, the processing of a substrate to produce a metabolite of interest. Once metagenomic libraries are constructed and grown in a plate-based format, a reporter strain may be added in co-culture. If the reporter strain is activated, the compound of interest will have had been secreted by the environmental DNA containing E. coli. The genes involved, which can comprise biosynthetic clusters, regulatory machinery and/or secretion apparatuses may be identified through transposon metagenesis and re-screening. These genes may then provide genetics scaffolds for engineering the desired production rate and titer needed for use in industrial batch fermentations.

To date, previously characterized reporters have been applied in various screening strategies. However, the approach described herein is unique, in that, the reporter constructs may be originally discovered through screening for compound-specific activation prior to interrogating metagenomic libraries. In one example, an E. coli library of GFP transcriptional fusions to approximately 2000 promoters on low copy plasmids was screened for substrate-induced expression using a pool of monocyclic aromatic acids. A single reporter was identified that regulates the emrRAB operon encoding a transcriptional regulator (emrR) and multidrug resistance pump (emrAB) for extrusion of toxic compounds. Previously, the expression of this resistance pump in E. coli was only known to be regulated by a small number of antibiotic substrates that do not include the compounds used in the screen. The substrate range of this reporter system was characterized and showed that sensitivity could be modulated via plasmid copy number. The reporter was then applied in screening a coal bed derived fosmid library before responsible genes were identified on selected clones and the compounds being secreted were identified by gas chromatography-mass spectrometry (GC-MS). Overall, the emrR reporter system described herein showed vast potential for use identifying metagenomic library clones useful in the production of fine chemicals relevant to both industrial and pharmaceutical applications.

Due to the iterative nature of the present methods, there are certain advantages to discover both the biosensor and the biosynthetic or catalytic operons of interest. Furthermore, some embodiments of the present methods may also address the problem of availability of MIEs and some embodiments also have the potential to address the inherent host compatibility problems associated with screening environmental DNA. The present methods may be iterative and thus more agile than prior art methods.

Uchiyama and Miyazaki⁵ ligate 7 kB fragments into a vector containing a promoter-less GFP. This was building from standard promoter trap methods that have been used in genetics for many years. The use of mobile genetic elements and large inserts (for example, more than 10 kB) give limitless combinatorial potential, agility and efficiency—to the extent that our method could possibly access every MIE that exists in prokaryotes. The same is not true for the Uchiyama and Miyazaki⁵ method.

The Uchiyama and Miyazaki⁵ method is dependent on ligation, restriction enzymes, and a static vector-based fluorescent marker. This puts both size limitations on the DNA fragments and excludes functioning components that happen to be downstream of the reporter. The size limitation is significant, since most (independent) genetic circuits in prokaryotes are organized into genomic architectures that range from 10 kB-500 kB (often called genomic islands). Further, the genetic logic that can be performed (to detect things) is constrained by the amount of gene products. Cloning large fragments still remains a very technically daunting and is inefficient, furthermore the Uchiyama and Miyazaki⁵ method does little to remedy this.

In terms of the static vector-based fluorescent marker, this not only prevents downstream functioning components, but also limits the probability of feedback loops (a very common circuit architecture). Accordingly, the genes are restricted to a linear orientation, whereby you can only insert the GFP in order going down the DNA strand. For example, in an operon, in order to capture all the functioning components of the operon, it would have to cut the detecting DNA right at the end before the terminator (a range of a few base pairs) or else interrupt the operon as you move toward the promoter. With a mobile genetic element (MGE), the marker can insert anywhere in the operon, whether it disrupts a gene or not.

Furthermore, the Uchiyama and Miyazaki⁵ method is locked in terms of directionality and number of markers. The MGE containing marker can insert in any direction with any number of other markers (or complimentary markers) into the same large insert. Thus demonstrating the possible combinations and possible permutations allowed for by the current methods.

A further benefit of using MGEs is that they can be non-biased or purposefully bias based on the flanking sequences. You can increase relative amounts of homologous recombination with your insertions (MGEs) and target different DNA properties or sequences. This could be as broad as to target specific GC contents or as specifically to target desired insertion sites. Since DNA synthesis is inexpensive, modifying MGEs in such as way becomes trivial and makes the present method much more agile.

MGEs enable all the retrofitting and MIE discovery steps in the method to be performed in vivo. Accordingly, DNA does not need to be cut up each time and re-cloned. Using the methods described herein, existing libraries may be retrofit in the cells they already reside in, which is much more efficient.

The transposon retrofitted MIE library method do not depend on restriction digestion as does SIGEX. Restriction digestion has several limitations, for example, if any regulators or machinery is downstream (beyond an operon and necessary for the MIE) SIGEX would miss it as GFP is the last gene in the construct. This would inherently limit what could be retrieved.

Furthermore, where the majority of proteins are not annotated with anything to do with the process being investigated, the current process differs significantly from PIGEX, which looks for known activities. Accordingly, the embodiments of the present method have the potential to identify “unknown” regulators, “unknown” pathways and “unknown” enzymes/cofactors etc. Furthermore, PIGEX adds a substrate that is one enzymatic conversion away from the step they are targeting. Doing this places limits on the ability to detect biosynthetic pathways (whether they comprise an operon, interact with host metabolism, or a segmented pathway). This is because the substrate creates a heavy selection against the preceding steps in the biosynthesis pathway. To make sure selection is for a biosynthetic pathway, there has to be careful considerations for the media (all substrates present) and the final product being detected.

Compatibility is also an issue, wherein the use of large insert libraries can be very powerful in overcoming this, identifying an MIE from a functional metagenomic library, examining the MIE for compatibility with the host strain, which may be selected by the MIE screen, since the same bacteria may be used in the MIE screen and metagenomic library screen.

One reason for using large insert libraries is to get an independent circuit. Otherwise, the components that sense the compound (say a transcription factor or signal transducer) would be limited to what is present in the host. Thus, if it worked in the screen, it is very unlikely to be incompatible with that host.

For example, a novel set of genes conferring the ability to secrete aromatics, including those that can be derived from lignin. The detection of heterogeneous aromatic secretion in growth media was identified using the emrR reporter system.

Furthermore, the system described has the potential to provide sustainable biological production of pure enantiomeric products. Such products could have decreased costs as compared to chemical synthesis. Furthermore, the methods described herein are promising for bioprospecting applications in the discovery of novel enzyme products for consumer and industrial markets. Furthermore, the number of potential diverse and often extreme environments that may be screened for novel microbial genes that may act as a rich source of material for novel enzyme products is somewhat limitless.

### Methods and Materials

Strains, plasmids and oligonucleotides used are set out below. Detailed procedures for construction of vectors, characterization of the PemrR-GFP biosensor and high-throughput screening are described in the Methods section. All DNA manipulations were performed according to standard procedures. Fosmid library preparation, transposon mutagenesis, and purification were performed with kits sourced from EpiCenter (Illumina™).

All chemicals used were of analytical grade and purchased from Sigma-Aldrich™ Monoaromatic lignin transformation products were identified by gas chromatography-mass spectrometry (GC-MS) as previously described.

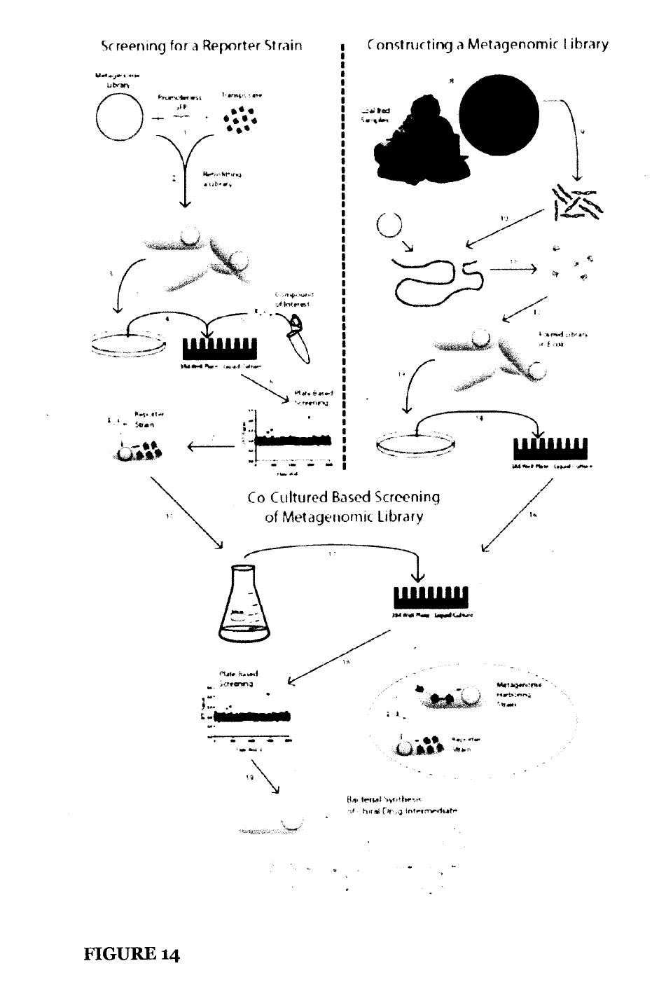

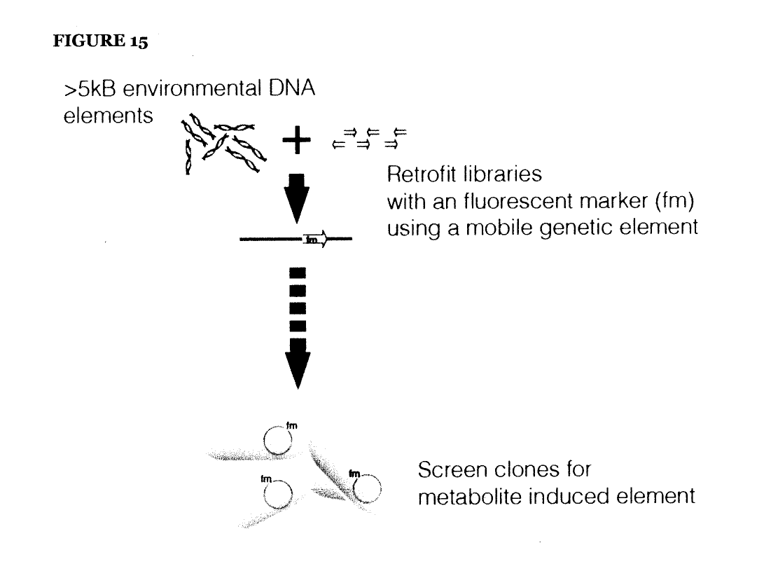

A graphic representation of an embodiment of the claimed method for (1) isolating metabolite induced elements (MIEs) from a metagenomic library, (2) construction of a metagenomic library, and (3) subsequently sequentially screening in co-culture, where the MIE is used in a reporter strain to act as a biosensor in the detection of meteagenomic elements producing heterologous metabolite secretion products from the functional metagenomic library (see FIG. 14 and FIG. 15). In FIG. 14, steps 1-3 show a random insertion of a mobile genetic element (for example, transposons) comprising a promoterless green fluorescent protein (gfp) gene into a metagenomic library to produce a metagenomic library retrofitted with a promoterless reporter. Steps 4-6 show screening with for a MIE using a metabolite of interest to obtain a reporter strain (step 7). In steps 8-14 a metagenomic library is assembled from bacterial samples obtained from a coal bed, but a person of skill in the art would appreciate that metagenomic libraries may be obtained from any number of sources depending on the metabolites of interest and samples that are available. Furthermore, the metagenomic library produced in steps 8-14 may be the same as the metagenomic library used to produce the metagenomic library retrofitted with a promoterless reporter of steps 1-7 or may be entirely different. In steps 15-18 a co-culture based screening is performed with the previously discovered reporter strain to select a metagenome element conferring metabolite secretion. Once the co-culture screening identifies one or more functional metagenomic library clones that produce the metabolite of interest, steps 1-7 may be repeated to identify additional metabolites of interest further down a pathway of interest as many times as needed. Alternatively as shown in step 19, the metabolite of interest may be produced by the clone or clones of interest for further characterization, study or as a source of the metabolite of interest.

### Strains and Growth Conditions

Bacterial strains, plasmids and primers are listed below. Minimal media consisted of M9 minimal media supplemented with glucose (0.4%), arabinose (100 μg ml-1), leucine (40 μg ml-1), MgSO4 (1 mM) and thiamine (2 μM). Lysogeny broth (LB) and minimal media were supplemented with Kanamycin (50 μg ml-1), Chloramphenicol (12.5 μg ml-1), and Ampicillin (100 μg ml-1) to maintain pUA66, PCC1fos and pBAD24, respectively. The emrR (JW2659-1), emrA (JW2660-1), and emrB (BW25112) knockout and cognate wild-type strains were obtained from the Keio collection through the Coli Genetic Stock Center (CGSC). All cultures were grown at 37° C. in a 220 r.p.m. rotary shaker unless otherwise stated.

### Plasmid Construction

The emrRAB promoter region (see the sequence below) and GFP were amplified from the pUA66 backbone with primers Frep (Promoter—EcoR1 (Forward) GCGGAATTCCGCAGCATTATCATCC) and Rrep (GFP—HindIII (Reverse) GCGAAGCTTCCTGCAGGTCTGGACATTTAT). The PCR product was digested with EcoRI and HindIII, and ligated with EcoRI/HindIII digested PCC1fos to generate a PCC1 reporter. The PCC1fos vector is under a copy-control system which is inducible in the EPI300 background host (EPICENTER™). PCCireporter under high-copy number in EPI300 is referred to as the PemrR-GFP biosensor. For inducible overexpression of emrR, emrR was amplified from E. coli K12 genomic DNA using the primers FemrR (emrR—EcoR1(Forward)GCGGAATTCatgGATAGTTCGTTTACGCCCA) and RemrR (emrR—HindIII (Reverse)GCGAAGCTTttaGCTCATCGCTTCGAGAACC). The resulting PCR product was also digested with EcoRI and HindIII, and ligated with EcoRI/HindIII digested pBAD24, yielding pBAD24emrR.

Sequence of the emrRAB Promoter

CGCAGCATTATCATCCCAACACTGCTTAGTGCGCTGGCCTATGGGCTCGC
CTGGAAAGTGATGGCGATTATATAACCCACAAGAATCATTTTTCTAAAAC
AATACATTTACTTTATTTGTCACTGTCGTTACTATATCGGCTGAAATTAA
TGAGGTCATACCCAAATGGATAGTTCGTTTACGCCCATTGAACAAATGCT
AAAATTTCGCGCCAGCCGCCACGAAGATTTTCCTT

Screening E. coli Reporter Library

A library of 1,820 E. coli K12 MC1655 intragenic regions fused to gfpmut2 on low copy plasmids was replicated into 96-well round-bottom culture plates containing M9 minimal medium supplemented with glucose. After growth overnight, a compound pool comprising 1 mM Vanillin, vanillic acid, p-coumaric acid, vanillyl alcohol and veratryl alcohol was added. The plates were then incubated and GFP fluorescence was measured by reading excitation at 481 nm and emission at 508 nm on a Varioscan Flash Spectral Scanning Multimode Reader (Thermo Scientific™) before selecting the most active clone (all GFP measurements were made as described here).

### PemrR-GFP Biosensor Characterization

The PemrR-GFP biosensor was grown overnight and diluted 1/10 in 180 μL of LB. The compound of interest, dissolved in 20 μL of 30% DMSO was then added before a 2 hr incubation and subsequent reading of GFP fluorescence. Arabinose was removed from the media when comparing the effect of plasmid copy number. For in vitro enzyme assays, PemrR-GFP was diluted 1/10 and incubated in M9 minimal medium with glucose (0.5%), 0.5 μg HKL-F1, manganese (40 mM), glucose oxidase (100 nM) and DypB N246A (50 nM). GFP measurements were made every 30 min.

### Environmental Isolate Screening

In vivo isolate cultures were carried out in 1/10 diluted low-salt (50 mg/L CaCl2*2H2O) LB, 1 g/L of HP-L™ and 1% DMSO that was filtered in a 0.2 μM filter (ExpressPlus™) (Millipore™). Cultures were inoculated 1/100 from saturated cultures grown in 1/10 diluted LB and grown in stationary flasks for 2 weeks before cells were centrifuged at 16,000×g and culture supernatant was removed and filtered with a 0.2 μM filter before being assayed with the PemrR-biosensor. The supernatant from duplicate cultures was aliquoted in 180 μL volumes in triplicate. To this, 20 μL of the PemrR-GFP biosensor, diluted 1/4 from an overnight culture in LB, was added and allowed to incubate stationary for 2 hrs before taking GFP measurements.

Gas Chromatography-Mass Spectrometry (GC-MS) Incubated with Lignin

Minimal media was stirring at 37° C. before 1 g/L of HP-L™ and HKL-F1 dissolved in DMSO (3% final DMSO) was added. The media (lignin amended media) was allowed to stir for 1 hr before being filtered through a 0.2 μM DMSO safe filter (ExpressPlus™ from Millipore™) to remove any precipitate. The EPI300 strains harboring fosmids were then inoculated 1/10,000 in 5 mL of lignin media from an overnight culture in LB. The cultures were allowed to grow for 16 hr before cells were spun down at 16,000×g for 10 min and culture supernatant was removed. The culture supernatant was acidified using formic acid (10% final concentration v/v). Acidification precipitated the residual lignin in all the clones, which was removed by centrifugation (16,000×g for 10 min) and filtration (0.2 μm DMSO safe filter—Pall™). The clear supernatant was extracted thrice using ethyl acetate (1:1). The extracts were dried over anhydrous magnesium sulfate and the solvent was evaporated under the stream of nitrogen. The air-dried samples were resuspended in 300 μl pyridine. To each of the sample, 4-chlorobenzoic acid (100 μg) was added as the internal standard. Subsequently, the samples were derivatized using BSTFA+TMCS− (99:1). GCMS was performed using an HP 66890 series GC system fitted with an HP5973 mass selective detector and a 30×250 μm HP-5MS Agilent™ column. The operating conditions were TGC (injector), 280° C.; TMS (ion source), 230° C.; oven time program (To min), 120° C.; T2 min, 120° C.; T45 min, 300° C. (heating rate 4° C. mini); and T54 min, 300° C. The injector volume was 1 μl.

### EmrRAB Characterization

All time course OD₆₀₀ and fluorescence measurements were made on an Infinite 200 PRO plate reader (TECAN™) with wild-type (BW25112), emrR(−) and emrB(−) E. coli strains. Monoaromatic compounds were added in DMSO to a final concentration of 3%. The pUA66 vector harboring the reporter construct was used in the BW25112 background for monitoring fluorescence. For studying the expression of emrR, pBAD24 expressing emrR was induced with 0.06 mM arabinose.

### Fosmid Library Production

A fosmid library was prepared from coal bed core samples provided by Alberta Innovates and DNA was extracted from the homogenized samples using previously described methods³⁰. The environmental DNA was cloned into the PCC1fos copy control vector and transformed into the EPI300 host (EPICENTER™) as previously described²¹. Both CO182 and CO183 samples yielded approximately 60,000 fosmid clones with an average insert size of 42 kB. Approximately 20,00 clones from each sample were Sanger end sequenced (Applied Biosystems 3730 system™) at Michael Smith's Genome Science Center (B.C., Canada) and metagenomes for the samples have been reported²⁰.

### High-Throughput Functional Screening

For fosmid library screening, 60,000 clone libraries were replicated using a Qpix2 robotic colony picker (Genetix™) in 384-well black plates. Clones were grown in 45 μL of LB for 12 hrs and 20 μL of LB containing HP-L™ (added as described in the GC-MS profile section) was then added for another 5 hr incubation. The PemrR-GFP biosensor was then added by diluting an overnight culture 1/4 and adding 20 μL and incubated for 3 hrs before florescent measurements were taken.

### Full Fosmid Sequencing

After 24 active clones were selected, fosmid DNA was extracted using the FosmidMax DNA preparation kit (EPICENTER™) according to the manufacturer's protocols. Contaminating E. coli DNA was removed using PlasmidSafe DNase™ (EPICENTER′). All DNA concentrations were determined using Quant-iT PicoGreen™ (Invitrogen™) and 500 ng of each fosmid was sent to Michael Smith's Genome Science Center (B.C., Canada) for sequencing on a Illumina GAIIx sequencer (Illumina™).

### Transposon Mutagenesis

For 11 of the active clones, a Tn5 transposon mutagenesis library was created using the EZ-Tn5 kan insertion kit (EPICENTER′). Approximately 384 mutants were arrayed for re-screening as described in the high-throughput screening section. Mutants were Sanger sequenced (Applied Biosystems 3730 system™) at Michael Smith's Genome Science Center (B.C., Canada) and activity was mapped to fosmid position using BLAST™. Statistically significant decreases in PemrR-GFP activity were then selected using a Z-score ratio.

### Bioinformatic Analyses

All open reading frames (ORFs) were determined using Prodigal (http://prodigal.ornl.gov/) and all ORFs were annotated using BLAST of NCBI nr databases. A custom perl script was designed that uses Circos (http://circos.ca) to visualize homology between fosmids. BLAST was used to map the location of the metagenomic reads (E-value cutoff of 1E-10) to the fosmid ORFs and custom python scripts were used to visualize the abundance of each ORF in the metagenome. Phylogenetic assignment binning was done using Sort-ITEMS (http://metagenomics.atc.tcs.com/binning/SOrt-ITEMS).

### EXAMPLES

### Example 1: Identification of Metabolite Induced Element (MIE) for Lignin Transformation Products

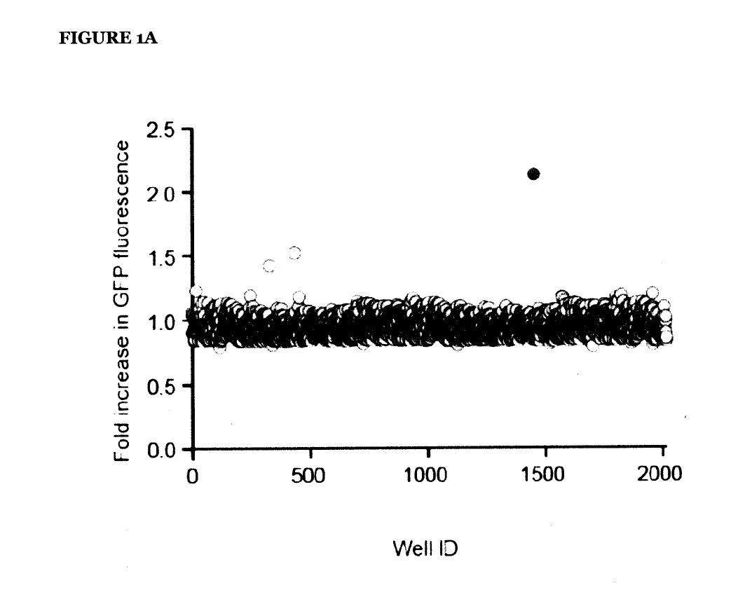

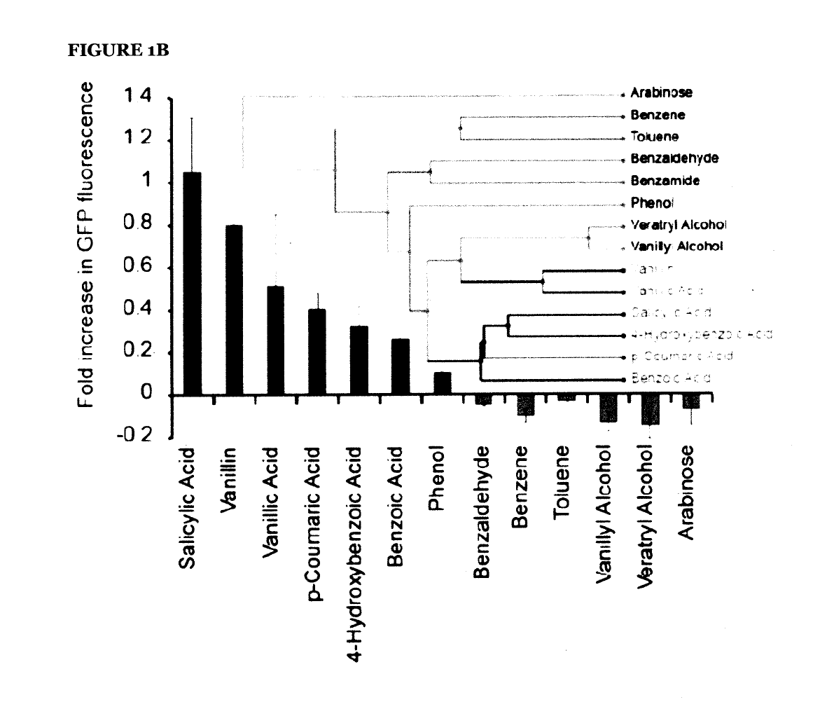

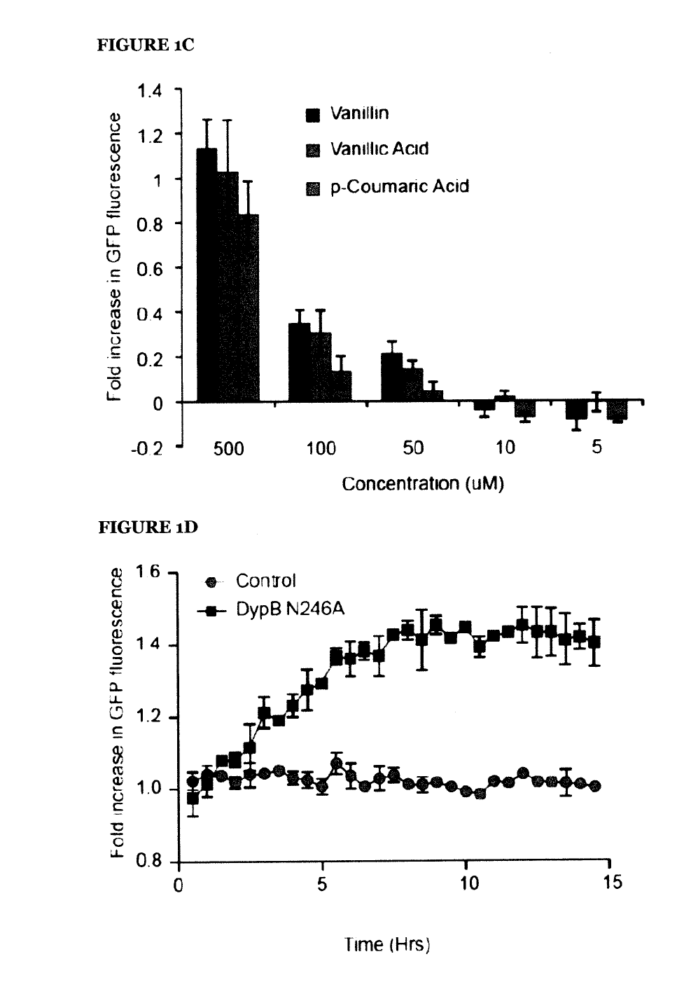

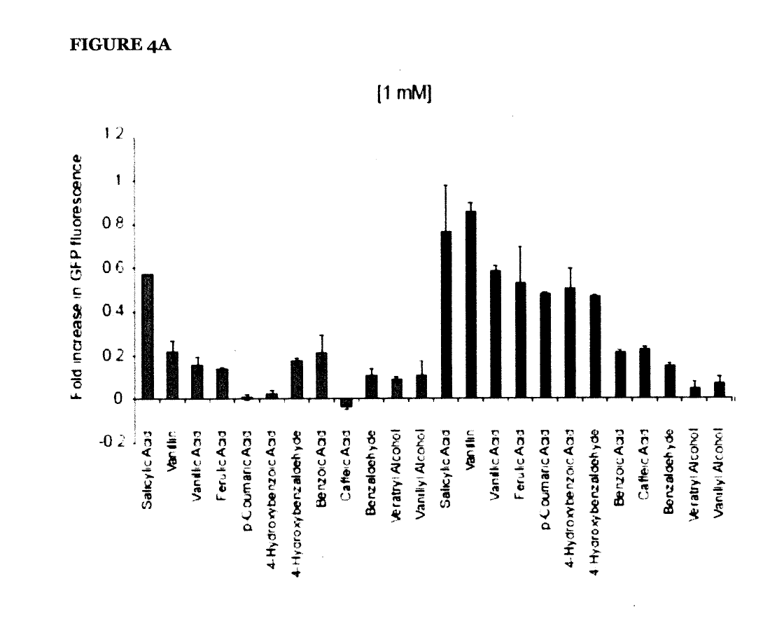

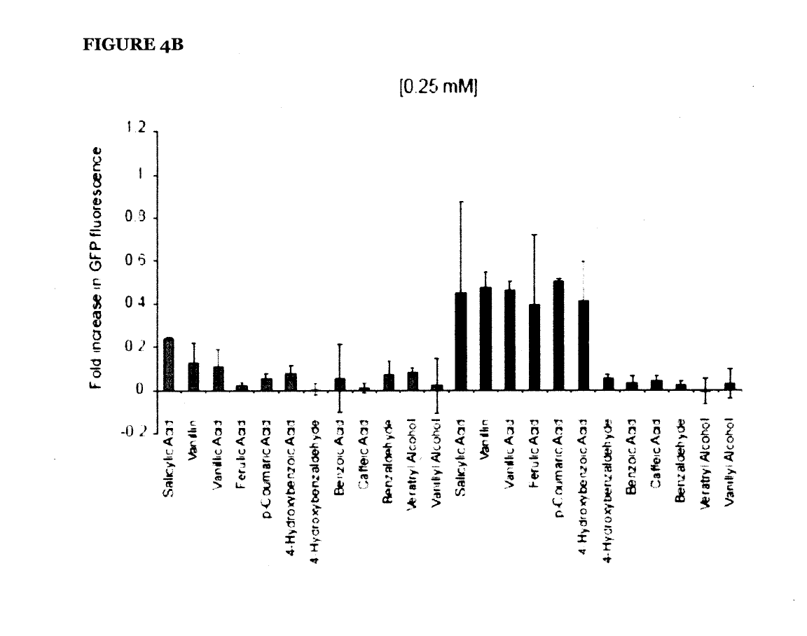

It was reasoned that sensing lignin transformation products rather than labeling the lignin polymer itself might improve signal detection across a wide range of substrate specificities. Accordingly, an E. coli clone library of fluorescent transcriptional reporters was interrogated with a mixture of lignin transformation products including vanillin, vanillic acid and p-coumaric acid (FIG. 1A)¹⁶. The most responsive clone harbored a promoter regulating the emrRAB operon, encoding a negative feedback transcriptional regulator (emrR) and multidrug resistance pump (emrAB) that is known to act on various structurally unrelated antibiotics^{17,18}. Since the compounds used to identify the emrR promoter were not previously been shown to induce emrRAB expression, response specificity was evaluated using a library of monoaromatic compounds (FIG. 1B). Sensitivity of detection was observed to increase with promoter copy number reaching a lower detection threshold of 50 μM using the three most active lignin transformation products (FIG. 4, FIG. 1C). The capacity of this promoter to detect in vitro lignin transformation was demonstrated by monitoring formation of monoaromatic products from a solvent fractionated hardwood kraft lignin (HKL-F1) using an engineered manganese-oxidizing dye decolorizing peroxidase (DypB N246A)¹⁰ (FIG. 1D).

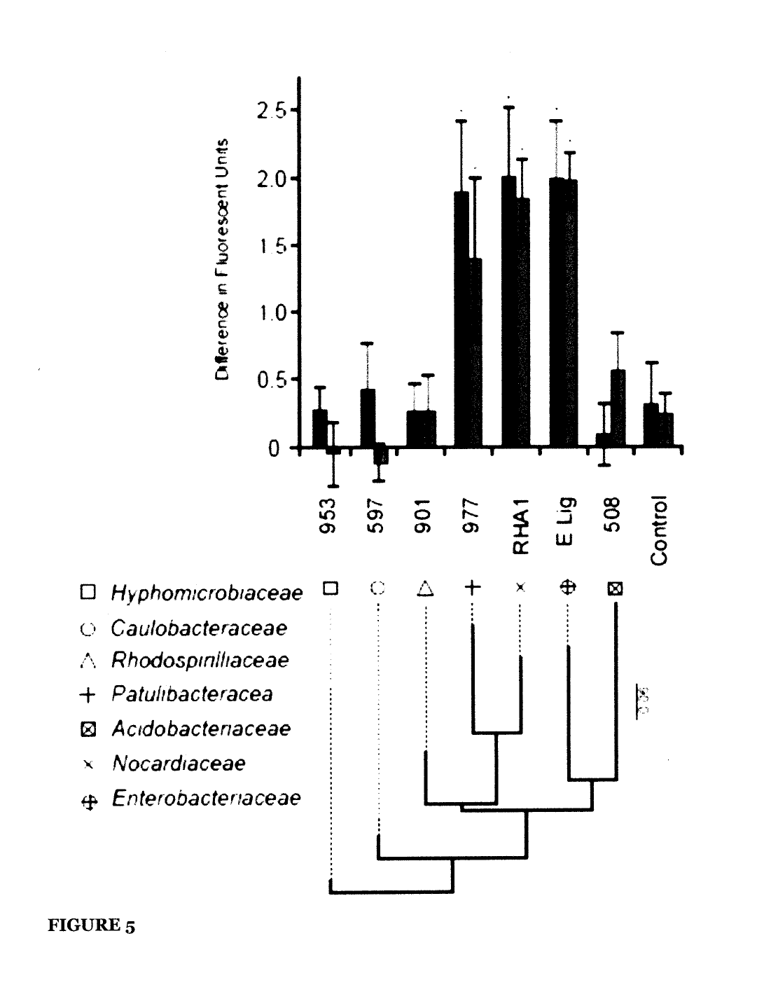

Co-culture based detection of lignin transformation products was also demonstrated using bacterial isolates affiliated with multiple phyletic groups, including Enterobacter lignolyticus SCF1 and Rhodococcus jostii RHA1 (FIG. 5).

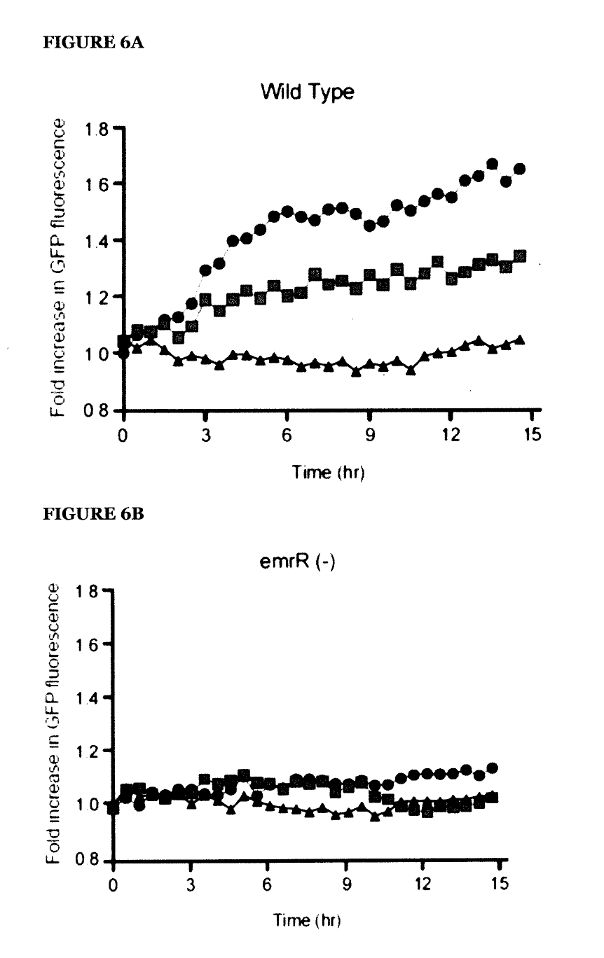

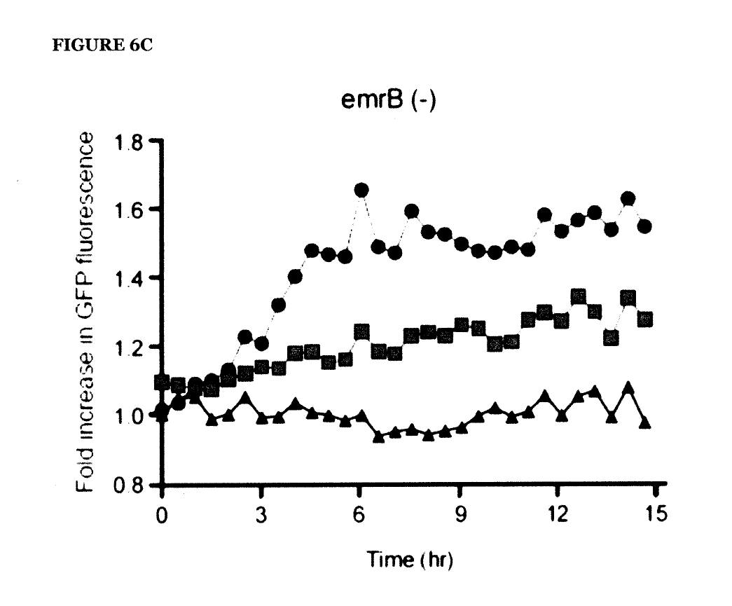

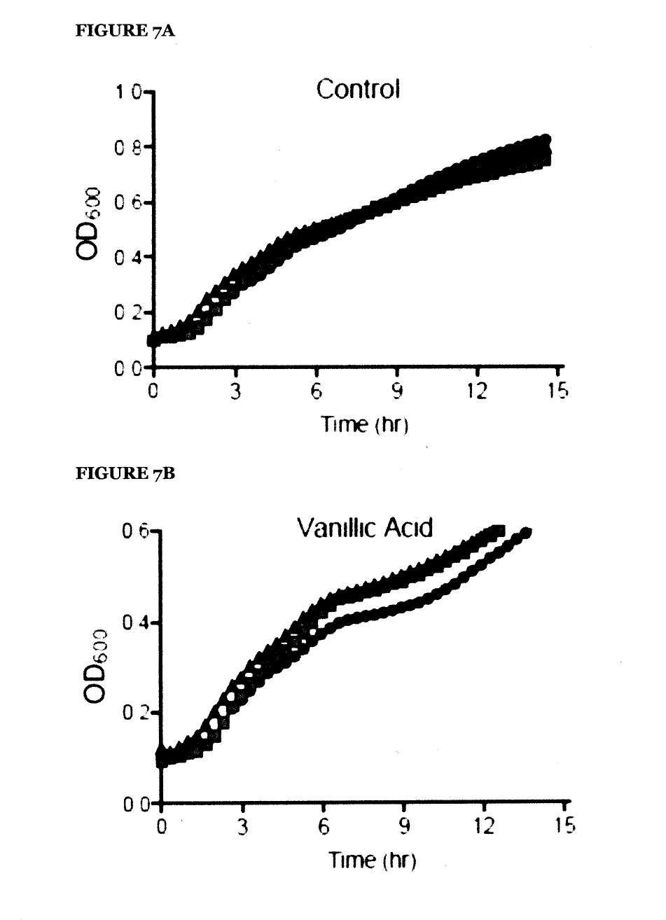

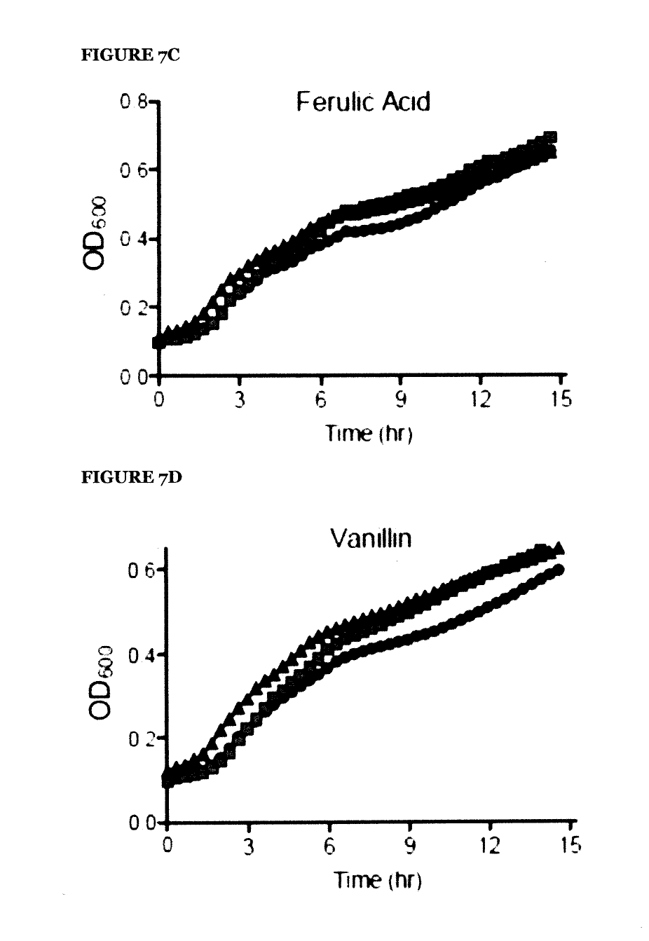

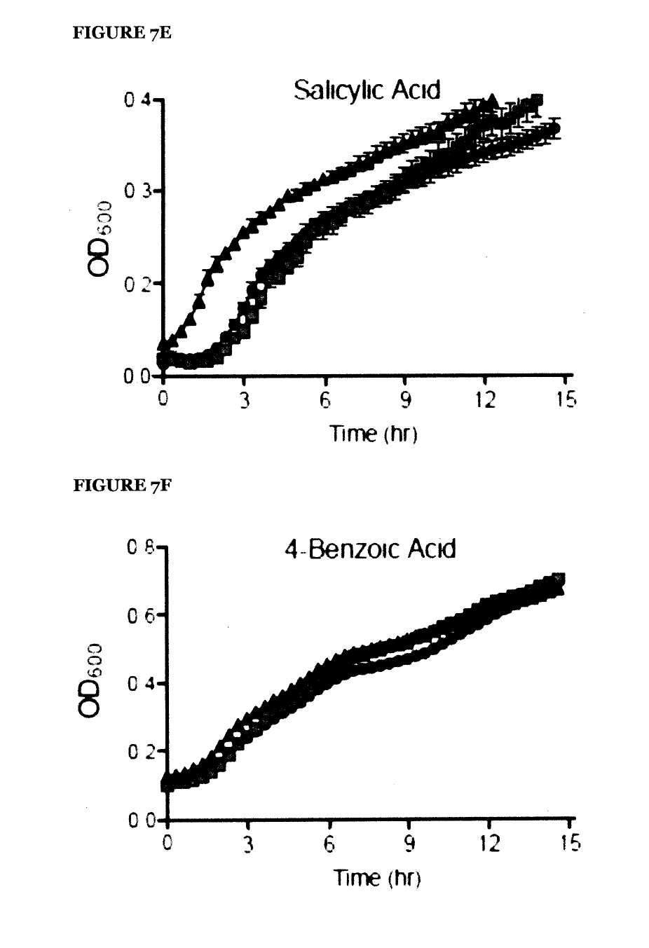

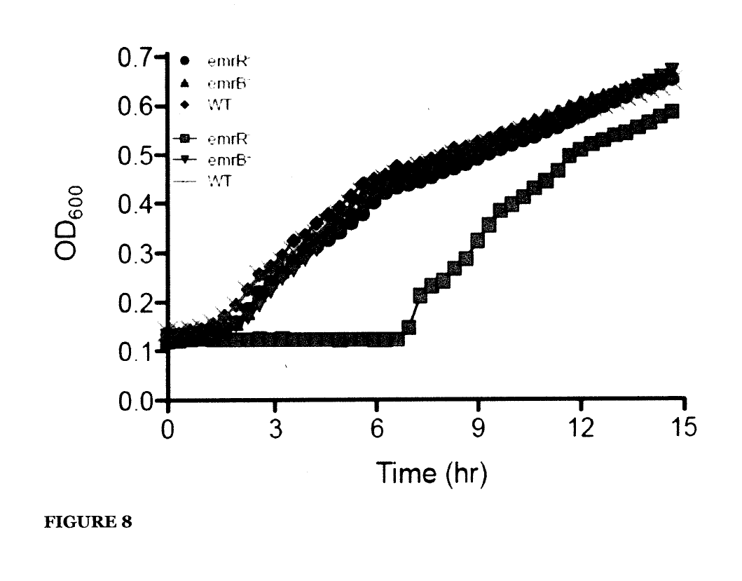

To evaluate the role of the EmrR transcriptional regulator in responding to lignin transformation products, it was shown that emrR is necessary and sufficient for the compound-dependent activation of the emrRAB operon (FIG. 6). Abolishing emrR, but not emrB activity caused slightly impaired growth kinetics in the presence of several lignin transformation products (FIG. 7). Moreover, a dramatic lag phase was consistently observed in emrR loss of function mutants exposed to HKL-F1 pretreated with DypB N246A (FIG. 8).

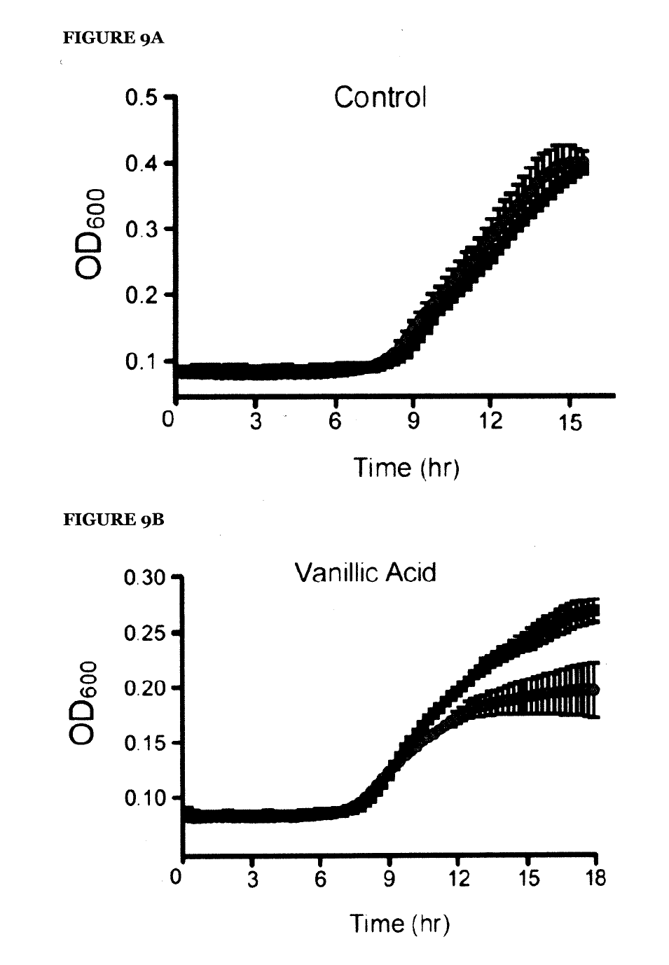

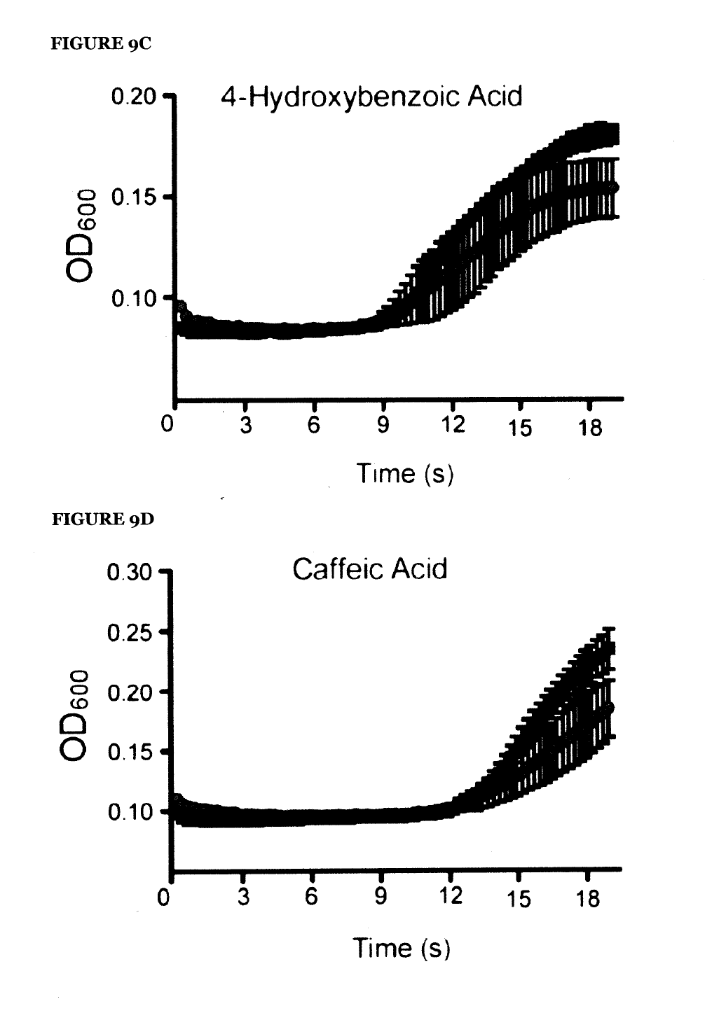

Complementation by over expression not only rescued the impaired growth phenotype in the presence of monoaromatic compounds, but also increased the growth rate and final biomass accumulation (FIG. 9). Taken together, these results are consistent with a role for EmrR in regulating an extended metabolic network responsive to monoaromatic exposure in the environment and reinforce the potential of using EmrR and its promoter as a versatile biosensor (PemrR-GFP) in functional screens for lignin transformation pathways.

### Example 2: Functional Metagenomic Library Screening

The structural composition of high molecular weight coal is derived from lignin but made increasingly recalcitrant through the processes of coalification¹⁹. It was reasoned that coal beds would be enriched for bacterial genes encoding lignin transformation pathways, where the primary transformation is not likely to be mediated by fungi^{9,20}, unlike forest soils. Therefore, bacterial dominated functional metagenomic libraries sourced from standard (CO182) and basal (CO183) coal formations in Alberta, Canada were interrogated for lignin transformation phenotypes using the PemrR-GFP biosensor²⁰. Metagenomic libraries from CO182 and CO183 were constructed using the Fosmid CopyControl system (pCC1FOS) from EpiCentre, as previous reports suggest that increased copy number enhances heterologous gene expression in the EPI300 E. coli host²¹. In parallel, the PemrR-GFP biosensor (a reporter strain) was transferred to the pCC1FOS vector used in library production to facilitate co-culture based screening using shared antibiotic selection. A total of 46,000 fosmids arrayed in 384-well plates were grown in the presence of HKL-F1 overnight prior to the addition of the biosensor.

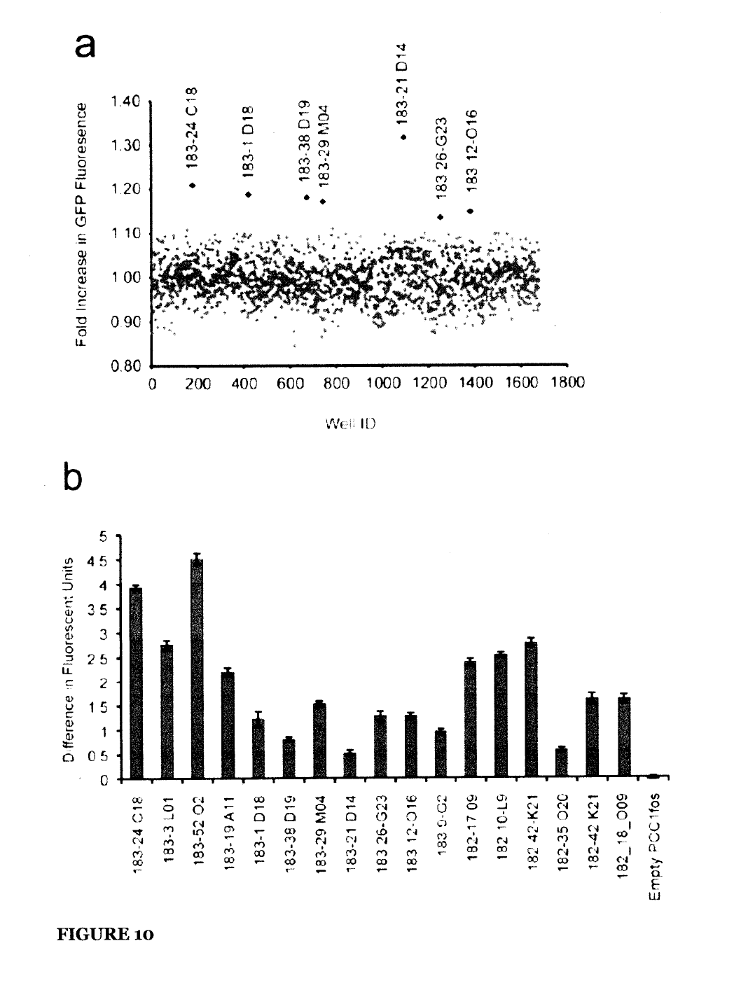

Co-cultures were subsequently grown for three hours prior to measuring GFP fluorescence. Fluorescent signals were normalized to background and corrected for edge effects. Consequently, 24 fosmids activating the emrR biosensor (16 from CO182 and 8 from CO183) were selected for downstream functional characterization and sequencing (FIG. 10).

### Example 3: Lignin Transformation Testing with Fosmids

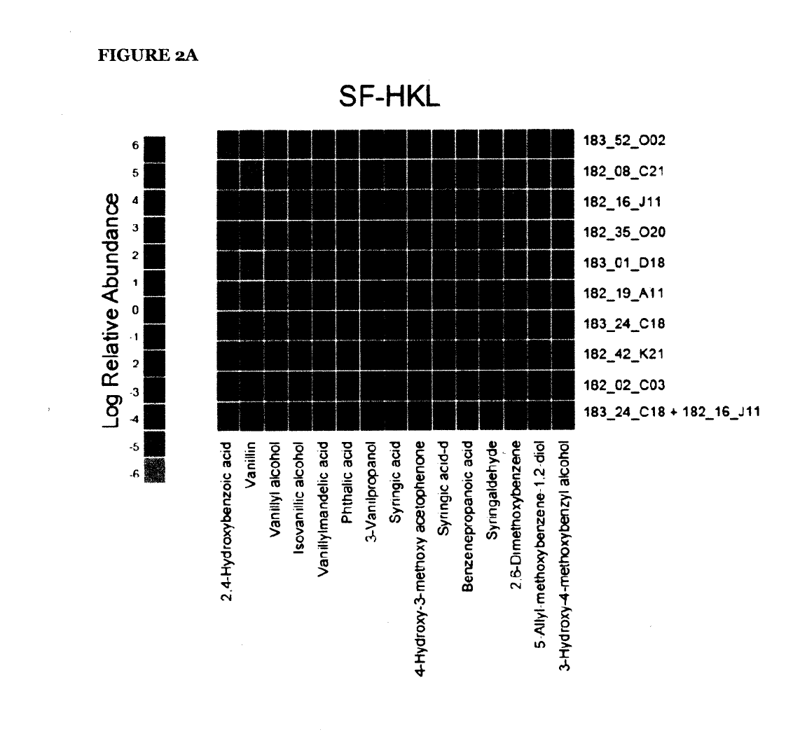

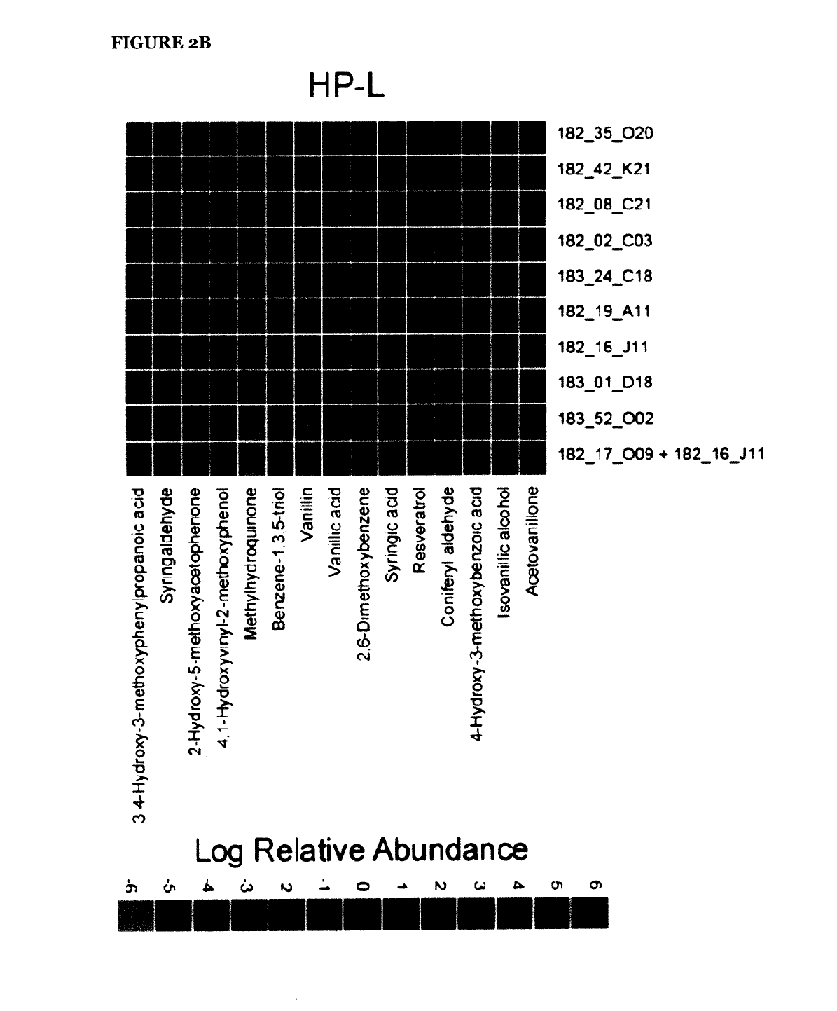

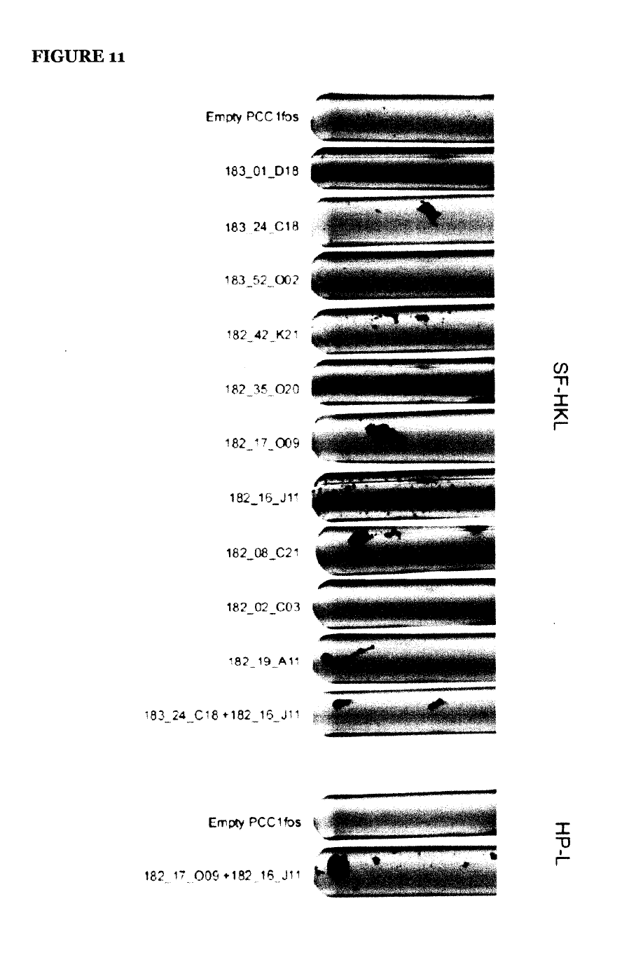

To verify the production of lignin transformation products by fosmids activating the PemrR-GFP biosensor, 11 of the most active clones were incubated in the presence of HKL-F1 and a second industrially purified high-performance lignin (HP-L™) substrate²². Lignin transformation products including vanillin, syringaldehyde and syringic acid were then measured by gas chromatography-mass spectrometry (GC-MS). An array of monoaromatic compound profiles were observed for single fosmid incubations, which varied between HKL-F1 and HP-L™ as consistent with different substrate properties or varying specificities of fosmid encoded enzymes (FIG. 2). Curiously, fosmid co-cultures exhibited synergy in combination, producing monoaromatic compound profiles that differed from individual fosmid incubation profiles in unexpected ways (FIG. 2). Moreover, while single fosmid incubations with HWKL F1 led to precipitate formation, only co-culture fosmid incubations were capable of forming precipitates with HP-L™. (FIG. 11).

The observations confirm that fosmids recovered in the PemrR-GFP biosensor screen confer lignin transformation phenotypes with different end product profiles, similar to observations made for fungal lignin transformation processes^{23,24}.

### Example 4: Gene Analysis

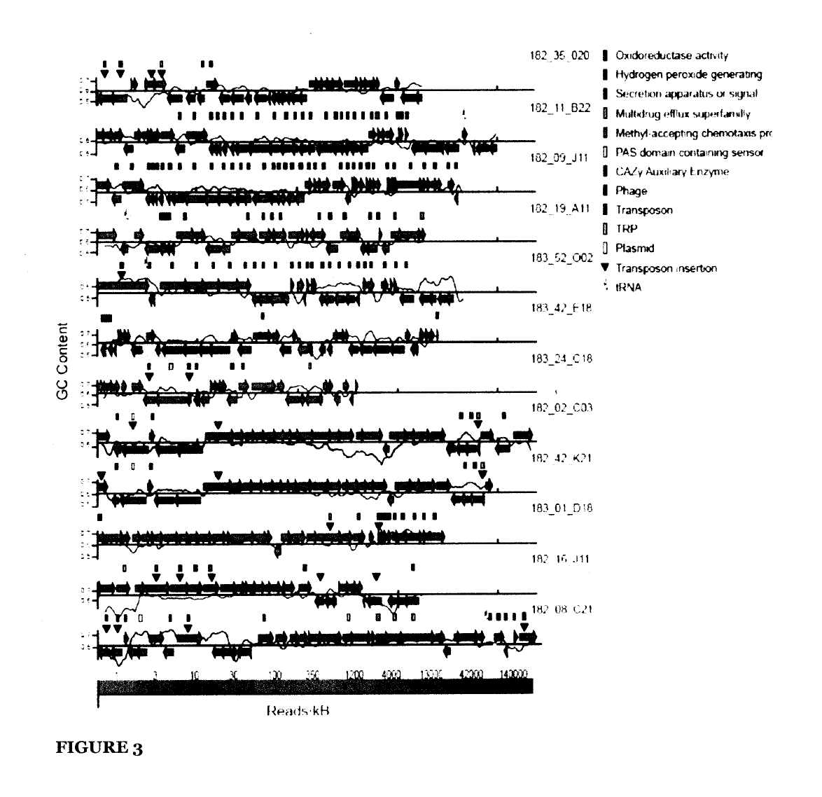

Random transposon mutagenesis identified genes encoded on the ii characterized fosmids necessary for activating the PemrR-GFP biosensor. Nine out of ii fosmids contained transposon insertions capable of reducing biosensor activation in two or more genes, suggesting that the observed lignin transforming phenotypes require multiple pathway components (FIG. 3).

Consistent with this observation, mapping the location of each transposon insertion identified six functional classes implicated in lignin transformation phenotypes. These included genes predicted to encode electron transfer (unassigned oxidoreductase activity), co-factor generation (hydrogen peroxide formation), protein secretion (secretion apparatus or signal peptide), small molecule transport (multidrug efflux superfamily), motility (methyl accepting chemotaxis proteins (MCP)), and signal transduction (PAS domain containing sensors) pathway components (FIG. 3). Full-fosmid sequencing and comparative analysis of all 24 fosmids activating the PemrR-GFP biosensor also identified recurring subsets of genes on typically non-syntenic clones encoding one or more of the six functional classes identified by transposon mutagenesis (FIG. 3).

While electron transfer, co-factor generation and protein secretion have well-defined roles in lignin transformation^{7,13}, the roles of the remaining three functional classes remain uncertain. It is notable that several of the fosmids identified with the PemrR-GFP biosensor actually encode small molecular transport systems similar to emrR and emrB, further reinforcing a role for these genes in regulating microbial responses to monoaromatic exposure in the environment (see TABLES 1A and 1B). Cell motility could then play a role in establishing optimal cell positioning along transformational gradients.

This relationship between lignin transformation and cell motility is highlighted by a recent study that observed an enrichment of MCP encoding genes and transcripts in wood feeding termites relative to dung-feeding termites²⁵.

Finally, signal transducers could play a role in mediating lignin substrate specificity among and between microbial groups and contribute to gradient formation. Indeed, recent cultivation-dependent studies using nitrated lignin substrates from Wheat, Miscanthus, and Pine identified alternative transformation phenotypes among and between bacterial and fungal isolates¹⁵. The necessity of genes encoding both MCP and signal transduction on the fosmids identified in this study directly implicates both of these functional classes in mediating lignin transformation phenotypes in the environment (FIG. 3).

In addition to the six functional classes described above, 16 of the 24 fully sequenced fosmids harbored mobile genetic elements (MGE). These elements were typically located proximal to one or more of the six functional classes suggesting a role for metabolic island or islet formation in propagating lignin transformation phenotypes in the environment (FIG. 3). To further explore the relationship between lignin transformation phenotypes and genomic island or islet formation coverage depth, G+C content variation and tRNA positioning on the active fosmids was examined (FIG. 3). Fragment recruitment of 500 million unassembled Illumina reads sourced from CO182 and CO183 environmental DNA identified abrupt changes in coverage depth in genomic intervals harboring MGE and one or more of the six functional classes consistent with island formation (FIG. 3). The presence of islands was further supported in 8 of the fosmids where coverage changes were associated with variation in median G+C composition or tRNA gene positioning (FIG. 3).

As genome regions, opposed to whole genomes, are more likely to sweep through populations, gene frequency can give insight into both the ecological and functional importance of environmental DNA²⁶. Islands and islets have been shown to transfer ecologically important traits throughout a habitat specific horizontal gene pool²⁷, with notable examples in symbiotic and marine ecosystems²⁶⁻²⁹. A number of environmental fosmids that confer lignin transformation phenotypes, share common enzymatic, regulatory and transport features, and display substantial evidence of horizontal gene transfer (HGT) were retrieved. Although the principle of rational engineering has driven the development of modern biorefinery systems, our results demonstrate the utility of exploiting ecological design principles to build a new generation of biorefining organisms through the use of naturally assembled genetic parts.

TABLE 2
Lignin Transformation Positive Clone Genes

| Gene ID | Annotation | Accession | Start | Stop | Signal | Class |
|---|---|---|---|---|---|---|
| 182_09_J11_1 | putative aminopeptidase 2 | YP_006457178.1 | 151 | 1440 | #N/A | #N/A |
| 182_09_J11_2 | NAD(P)(H)-dependent oxidoreductase | EHY79679.1 | 1513 | 2475 | #N/A | Oxido |
| 182_09_J11_3 | prc gene product | YP_005938086.1 | 2649 | 4733 | #N/A | #N/A |
| 182_09_J11_4 | TPR repeat, SEL1 subfamily protein | YP_006457174.1 | 4905 | 5372 | #N/A | #N/A |
| 182_09_J11_5 | hypothetical protein PSTAB_1345 | YP_004713715.1 | 5372 | 5743 | #N/A | #N/A |
| 182_09_J11_6 | Cro/CI family transcriptional regulator | EIK53833.1 | 5740 | 6066 | #N/A | #N/A |
| 182_09_J11_7 | hypothetical protein A458_07510 | YP_006457171.1 | 6223 | 6699 | #N/A | #N/A |
| 182_09_J11_8 | helix-hairpin-helix repeat-containing compet | YP_006523711.1 | 7028 | 7342 | #N/A | #N/A |
| 182_09_J11_9 | flagellar hook-associated protein FlgL | YP_006523710.1 | 7463 | 8731 | #N/A | #N/A |
| 182_09_J11_10 | flagellar hook-associated protein FlgK | YP_004713710.1 | 8744 | 10759 | SEC | #N/A |
| 182_09_J11_11 | flagellar rod assembly protein/muramidase | YP_006457167.1 | 10763 | 11935 | #N/A | Secretion |
| | Fl | | | | | |
| 182_09_J11_12 | flagellar basal body P-ring protein | YP_006457166.1 | 11946 | 13046 | SEC | Secretion |
| 182_09_J11_13 | flagellar basal body L-ring protein | YP_004713707.1 | 13061 | 13756 | SEC | Secretion |
| 182_09_J11_14 | flagellar basal body rod protein FlgG | YP_006457164.1 | 13841 | 14626 | #N/A | Secretion |
| 182_09_J11_15 | flagellar basal body rod protein FlgF | YP_006457163.1 | 14662 | 15402 | SEC | Secretion |
| 182_09_J11_16 | flagellar hook protein FlgE | YP_006523703.1 | 15598 | 17184 | SEC | Secretion |
| 182_09_J11_17 | flagellar basal body rod modification protei | YP_004713703.1 | 17214 | 17897 | #N/A | Secretion |
| 182_09_J11_18 | flagellar basal body rod protein FlgC | YP_004713702.1 | 17917 | 18360 | SEC | Secretion |
| 182_09_J11_19 | flagellar basal body rod protein FlgB | YP_004713701.1 | 18372 | 18836 | #N/A | Secretion |
| 182_09_J11_20 | chemotaxis protein methyltransferase CheR | YP_006457158.1 | 18972 | 19796 | #N/A | MACP |
| 182_09_J11_21 | chemotaxis protein CheV | YP_004713699.1 | 19831 | 20763 | #N/A | MACP |
| 182_09_J11_22 | flagellar basal body P-ring biosynthesis pro | YP_001171918.1 | 20855 | 21595 | SEC | Secretion |
| 182_09_J11_23 | negative regulator of flagellin synthesis Fl | YP_006457155.1 | 21709 | 22038 | #N/A | Secretion |
| 182_09_J11_24 | FlgN family protein | EHY75837.1 | 22074 | 22544 | #N/A | Secretion |
| 182_09_J11_25 | type IV pilus assembly PilZ | YP_005938063.1 | 22604 | 23350 | #N/A | Secretion |
| 182_09_J11_26 | phage integrase family site specific | ZP_10640976.1 | 23619 | 24830 | #N/A | #N/A |
| | recombinase | | | | | |
| 182_09_J11_27 | hypothetical protein PMI32_04729 | ZP_10640977.1 | 24827 | 25399 | #N/A | #N/A |
| 182_09_J11_28 | excisionase | YP_001350480.1 | 25528 | 25728 | #N/A | #N/A |
| 182_09_J11_30 | hypothetical protein G1E_09582 | ZP_08139536.1 | 25862 | 26206 | #N/A | #N/A |
| 182_09_J11_31 | virulence-associated protein E | YP_001350484.1 | 26260 | 26727 | #N/A | #N/A |
| 182_09_J11_35 | hypothetical protein PfraA_21814 | ZP_10850479.1 | 27828 | 28385 | #N/A | #N/A |
| 182_09_J11_36 | hypothetical protein PMI22_00482 | ZP_10695917.1 | 29037 | 29210 | #N/A | #N/A |
| 182_09_J11_37 | hypothetical protein PMI22_00494 | ZP_10695929.1 | 29207 | 29479 | #N/A | #N/A |
| 182_09_J11_41 | hypothetical protein | YP_001667999.1 | 30075 | 32627 | #N/A | #N/A |
| 182_09_J11_45 | possible bacteriophage terminase small | ZP_04979132.1 | 34092 | 34493 | #N/A | #N/A |
| | subuni | | | | | |
| 182_09_J11_46 | resolvase | ZP_10150764.1 | 35126 | 35752 | #N/A | #N/A |
| 182_02_C03_1 | acyl-CoA dehydrogenase | YP_006456114.1 | 1 | 1266 | #N/A | #N/A |
| 182_02_C03_2 | peptide methionine sulfoxide reductase | YP_006456115.1 | 1681 | 2328 | SEC | Oxido |
| 182_02_C03_3 | sensory box protein PAS/PAC and GAF | YP_006456116.1 | 2440 | 5112 | #N/A | PAS |
| | sensor-containing | | | | | |
| 182_02_C03_4 | TPR repeat-containing protein | YP_006456117.1 | 5223 | 5750 | #N/A | #N/A |
| 182_02_C03_5 | pyruvate dehydrogenase | YP_006456118.1 | 5838 | 7844 | #N/A | #N/A |
| | dihydrolipoyltransace | | | | | |
| 182_02_C03_6 | 2-oxo-acid dehydrogenase E1 subunit | YP_006456119.1 | 7872 | 10517 | #N/A | #N/A |
| 182_02_C03_7 | bifunctional glutamine-synthetase adenylyltr | YP_006456120.1 | 10784 | 13729 | #N/A | #N/A |
| 182_02_C03_8 | branched-chain amino acid aminotransferase | EHY79420.1 | 13780 | 14703 | #N/A | #N/A |
| 182_02_C03_9 | lipopolysaccharide heptosyltransferase II | YP_006456122.1 | 14778 | 15812 | #N/A | #N/A |
| 182_02_C03_10 | lipopolysaccharide heptosyltransferase I | YP_006456123.1 | 15813 | 16814 | #N/A | #N/A |
| 182_02_C03_11 | UDP-glucose:(heptosyl) LPS alpha 1,3- | YP_006456124.1 | 16814 | 17935 | #N/A | #N/A |
| | glucosy | | | | | |
| 182_02_C03_12 | lipopolysaccharide core heptose(I) kinase | EHY79416.1 | 17979 | 18785 | #N/A | #N/A |
| | RfaP | | | | | |
| 182_02_C03_13 | lipopolysaccharide kinase | YP_005940536.1 | 18785 | 19519 | #N/A | #N/A |
| 182_02_C03_14 | lipopolysaccharide kinase | YP_006456127.1 | 19516 | 20259 | #N/A | #N/A |
| 182_02_C03_15 | serine/threonine protein kinase | YP_006456128.1 | 20259 | 21704 | #N/A | #N/A |
| 182_02_C03_16 | carbamoyltransferase | YP_006456129.1 | 21717 | 23471 | #N/A | #N/A |
| 182_02_C03_17 | glycosyl transferase family protein | YP_006456130.1 | 23458 | 24375 | #N/A | #N/A |
| 182_02_C03_18 | hypothetical protein A458_02260 | YP_006456131.1 | 24390 | 25619 | #N/A | #N/A |
| 182_02_C03_19 | capsule polysaccharide biosynthesis | YP_006456132.1 | 25874 | 26743 | #N/A | #N/A |
| 182_02_C03_20 | O-antigen polymerase protein | YP_006456133.1 | 26740 | 28497 | #N/A | #N/A |
| 182_02_C03_21 | toluene tolerance protein | YP_006456135.1 | 28520 | 29122 | #N/A | #N/A |
| 182_02_C03_22 | transport protein MsbA | YP_006456136.1 | 29158 | 30975 | #N/A | #N/A |
| 182_02_C03_23 | Mig-14 family protein | YP_006456137.1 | 30975 | 31871 | #N/A | #N/A |
| 182_02_C03_24 | LmbE family protein | YP_006456138.1 | 31875 | 33269 | #N/A | #N/A |
| 182_02_C03_25 | bifunctional heptose 7-phosphate | EHY79406.1 | 33347 | 34768 | #N/A | #N/A |
| | kinase/heptose 1 | | | | | |
| 182_02_C03_26 | hypothetical protein PstZobell_18470 | EHY79405.1 | 34839 | 35726 | #N/A | #N/A |
| 182_02_C03_27 | aldo/keto reductase family oxidoreductase | YP_006456141.1 | 35817 | 36626 | #N/A | HPG |
| 182_02_C03_28 | oxidoreductase, FAD-binding protein | EHY79403.1 | 36623 | 37798 | #N/A | Oxido |
| 182_02_C03_29 | multidrug efflux SMR transporter | YP_006456143.1 | 37859 | 38191 | TM | MDES |
| 182_02_C03_30 | 3-deoxy-D-manno-octulosonic-acid | YP_001174283.1 | 38311 | 39579 | #N/A | #N/A |
| | transferase | | | | | |
| 182_02_C03_31 | outer membrane efflux protein TolC/Type 1 | YP_001174282.1 | 39753 | 41207 | SEC | Secretion |
| | secretion | | | | | |
| 182_02_C03_32 | thiamine biosynthesis protein ThiC | EHY79400.1 | 41590 | 43497 | #N/A | #N/A |
| 182_08_C21_1 | site-specific recombinase, phage integrase fa | ZP_07104809.1 | 1118 | 1465 | #N/A | #N/A |
| 182_08_C21_2 | hypothetical protein CLOSCI_03331 | ZP_02433069.1 | 1684 | 2343 | #N/A | #N/A |
| 182_08_C21_3 | general secretion pathway protein F | ZP_09329114.1 | 2693 | 3838 | #N/A | Secretion |
| 182_08_C21_4 | luciferase family oxidoreductase | ZP_08950429.1 | 3915 | 5096 | #N/A | Oxido |
| 182_08_C21_5 | methyl-accepting chemotaxis sensory | ZP_09329117.1 | 5112 | 5597 | #N/A | MACP |
| | transduce.. | | | | | |
| 182_08_C21_6 | response regulator of the LytR/AlgR family | ZP_10389812.1 | 5714 | 6478 | #N/A | #N/A |
| 182_08_C21_7 | integral membrane sensor signal | ZP_08948803.1 | 6459 | 7580 | TM | PAS |
| | transduction | | | | | |
| 182_08_C21_8 | argininosuccinate lyase | ZP_04765045.1 | 7632 | 9089 | #N/A | #N/A |
| 182_08_C21_9 | catalase | ZP_08948807.1 | 9173 | 10225 | SEC | HPG |
| 182_08_C21_10 | large extracellular alpha-helical protein | ZP_10389820.1 | 10420 | 12924 | SEC | Secretion |
| 182_08_C21_11 | phosphoserine aminotransferase | EHY77425.1 | 14078 | 14905 | #N/A | #N/A |
| 182_08_C21_12 | phosphoserine aminotransferase | EHY77426.1 | 14898 | 15998 | #N/A | #N/A |
| 182_08_C21_13 | hypothetical protein YO5_08308 | EEK51406.1 | 15979 | 16482 | #N/A | #N/A |
| 182_08_C21_14 | pyrroloquinoline quinone biosynthesis protei | YP_006524654.1 | 16568 | 17713 | #N/A | #N/A |
| 182_08_C21_15 | pyrroloquinoline quinone biosynthesis protei | YP_006457741.1 | 17682 | 17978 | #N/A | #N/A |
| 182_08_C21_16 | aldehyde dehydrogenase | YP_006457739.1 | 18520 | 20040 | #N/A | Oxido |
| 182_08_C21_17 | NADH:flavin oxidoreductase/NADH | YP_006457738.1 | 20285 | 21403 | #N/A | Oxido |
| | oxidase | | | | | |
| 182_08_C21_18 | pyrroloquinoline quinone biosynthesis protei | YP_006457736.1 | 21700 | 22611 | #N/A | #N/A |
| 182_08_C21_19 | pyrroloquinoline quinone biosynthesis protei | YP_004714338.1 | 22697 | 23449 | #N/A | #N/A |
| 182_08_C21_20 | pyrroloquinoline quinone biosynthesis | EHY79205.1 | 23543 | 23821 | #N/A | #N/A |
| | protein Pqq | | | | | |
| 182_08_C21_21 | pyrroloquinoline quinone biosynthesis protei | YP_006457733.1 | 23793 | 24947 | #N/A | #N/A |
| 182_08_C21_22 | prolyl oligopeptidase family protein | YP_006457732.1 | 24944 | 26857 | #N/A | #N/A |
| 182_08_C21_23 | iron-containing alcohol dehydrogenase | YP_005938743.1 | 26965 | 28128 | #N/A | #N/A |
| 182_08_C21_24 | PAS/PAC sensor hybrid histidine kinase | EHY79209.1 | 28112 | 29809 | #N/A | PAS |
| 182_08_C21_25 | hypothetical protein PstZobell_17449 | EHY79210.1 | 29861 | 30205 | #N/A | #N/A |
| 182_08_C21_26 | CzcC family heavy metal RND efflux outer | EHY79211.1 | 30298 | 31551 | SEC | MDES |
| | membrane | | | | | |
| 182_08_C21_27 | CzcB family heavy metal RND efflux | EHY79212.1 | 31548 | 33035 | SEC | MDES |
| | membrane fusio | | | | | |
| 182_08_C21_28 | CzcA family heavy metal RND efflux | EHY79213.1 | 33032 | 36154 | SEC | MDES |
| | protein | | | | | |
| 182_08_C21_29 | Co/Zn/Cd efflux system protein | YP_006459952.1 | 36268 | 37149 | #N/A | #N/A |
| 182_08_C21_30 | hypothetical protein PstZobell_17469 | EHY79214.1 | 37158 | 37874 | #N/A | #N/A |
| 182_08_C21_31 | DNA-binding response regulator GacA | YP_006457724.1 | 38081 | 38725 | #N/A | #N/A |
| 182_08_C21_32 | excinuclease ABC subunit C | YP_004714350.1 | 38725 | 40548 | #N/A | #N/A |
| 182_08_C21_33 | CDP-diacylglycerol-glycerol-3-phosphate | YP_001172597.1 | 40582 | 41139 | #N/A | #N/A |
| | 3-p | | | | | |
| 182_08_C21_34 | Putative integrase | EEK53995.1 | 41509 | 42912 | #N/A | #N/A |
| 182_08_C21_35 | thiol:disulfide interchange protein precursor | EEK53996.1 | 43055 | 44821 | #N/A | #N/A |
| 182_08_C21_36 | metal-binding protein | YP_004714879.1 | 44972 | 45430 | #N/A | #N/A |
| 182_08_C21_37 | copper-binding protein | EHY76266.1 | 45456 | 45728 | #N/A | #N/A |
| 182_08_C21_38 | copper-translocating P-type ATPase | EHY76267.1 | 45803 | 48124 | #N/A | #N/A |
| 182_08_C21_39 | hypothetical protein PstZobell_02371 | EHY76268.1 | 48373 | 48675 | #N/A | #N/A |
| 182_08_C21_40 | ferredoxin | EHY76269.1 | 48742 | 49071 | #N/A | #N/A |
| 182_08_C21_41 | sensor protein CopS | YP_004714875.1 | 49226 | 50632 | #N/A | #N/A |
| 182_08_C21_42 | transcriptional activator CopR | EHY76271.1 | 50629 | 50964 | #N/A | #N/A |
| 182_08_C21_43 | ISPssy, transposase | YP_006456860.1 | 51048 | 52028 | #N/A | #N/A |
| 182_08_C21_44 | transcriptional activator CopR | EHY76271.1 | 52163 | 52501 | #N/A | #N/A |
| 182_08_C21_45 | blue (type1) copper domain-containing | EHY76272.1 | 53011 | 53388 | #N/A | Oxido |
| | protein | | | | | |
| 182_08_C21_46 | copper resistance protein A/twin-arginine | EHY76273.1 | 53574 | 55274 | TAT | Oxido |
| | translocation pathway signal | | | | | |
| 182_11_B22_1 | lipoprotein | YP_004713730.1 | 1 | 972 | #N/A | #N/A |
| 182_11_B22_2 | surface lipoprotein | YP_006457186.1 | 965 | 1732 | #N/A | #N/A |
| 182_11_B22_3 | pirin-like protein | EHY79687.1 | 1825 | 2667 | TAT | Secretion |
| 182_11_B22_4 | lipid A biosynthesis lauroyl acyltransferase | EHY79686.1 | 2713 | 3651 | #N/A | #N/A |
| 182_11_B22_5 | septum formation inhibitor | YP_006457183.1 | 3810 | 4535 | #N/A | #N/A |
| 182_11_B22_6 | septum site-determining protein MinD | YP_004713725.1 | 4631 | 5446 | #N/A | #N/A |
| 182_11_B22_7 | cell division topological specificity factor | YP_004713724.1 | 5443 | 5700 | #N/A | #N/A |
| 182_11_B22_8 | ribosomal large subunit pseudouridine | EHY79682.1 | 5762 | 6397 | #N/A | #N/A |
| | synthase A | | | | | |
| 182_11_B22_9 | putative aminopeptidase 2 | EHY79680.1 | 6517 | 7806 | #N/A | #N/A |
| 182_11_B22_10 | NAD(P)(H)-dependent oxidoreductase | YP_006457177.1 | 7879 | 8841 | #N/A | Oxido |
| 182_11_B22_11 | periplasmic tail-specific protease | YP_005938086.1 | 9015 | 11099 | #N/A | #N/A |
| 182_11_B22_12 | TPR repeat, SEL1 subfamily protein | YP_006457174.1 | 11271 | 11738 | #N/A | #N/A |
| 182_11_B22_13 | hypothetical protein PSTAB_1345 | YP_004713715.1 | 11738 | 12106 | #N/A | #N/A |
| 182_11_B22_14 | Cro/CI family transcriptional regulator | EIK53833.1 | 12103 | 12429 | #N/A | #N/A |
| 182_11_B22_15 | hypothetical protein A458_07510 | YP_006457171.1 | 12586 | 13062 | #N/A | #N/A |
| 182_11_B22_16 | helix-hairpin-helix repeat-containing compet | YP_006523711.1 | 13390 | 13704 | #N/A | #N/A |
| 182_11_B22_17 | flagellar hook-associated protein FlgL | YP_006523710.1 | 13825 | 15093 | #N/A | Secretion |
| 182_11_B22_18 | flagellar hook-associated protein FlgK | YP_004713710.1 | 15106 | 17121 | SEC | Secretion |
| 182_11_B22_19 | flagellar rod assembly protein/muramidase | YP_004713709.1 | 17125 | 18297 | #N/A | Secretion |
| | Fl | | | | | |
| 182_11_B22_20 | flagellar basal body P-ring protein | YP_006457166.1 | 18308 | 19408 | SEC | Secretion |
| 182_11_B22_21 | flagellar basal body L-ring protein | YP_004713707.1 | 19423 | 20118 | SEC | Secretion |
| 182_11_B22_22 | flagellar basal body rod protein FlgG | YP_006457164.1 | 20203 | 20988 | #N/A | Secretion |
| 182_11_B22_23 | flagellar basal body rod protein FlgF | YP_006457163.1 | 21024 | 21764 | SEC | Secretion |
| 182_11_B22_24 | flagellar hook protein FlgE | YP_006523703.1 | 21960 | 23546 | SEC | Secretion |
| 182_11_B22_25 | flagellar basal body rod modification protei | YP_004713703.1 | 23576 | 24259 | #N/A | Secretion |
| 182_11_B22_26 | flagellar basal body rod protein FlgC | YP_004713702.1 | 24279 | 24722 | SEC | Secretion |
| 182_11_B22_27 | flagellar basal body rod protein FlgB | YP_004713701.1 | 24734 | 25198 | #N/A | Secretion |
| 182_11_B22_28 | chemotaxis protein methyltransferase CheR | YP_006457158.1 | 25334 | 26158 | #N/A | MACP |
| 182_11_B22_29 | chemotaxis protein CheV | YP_004713699.1 | 26193 | 27125 | #N/A | MACP |
| 182_11_B22_30 | flagellar basal body P-ring biosynthesis pro | YP_001171918.1 | 27217 | 27957 | SEC | Secretion |
| 182_11_B22_31 | negative regulator of flagellin synthesis Fl | YP_006457155.1 | 28071 | 28400 | #N/A | Secretion |
| 182_11_B22_32 | FlgN family protein | EHY75837.1 | 28436 | 28906 | #N/A | Secretion |
| 182_11_B22_33 | type IV pilus assembly PilZ | YP_005938063.1 | 28966 | 29712 | #N/A | Secretion |
| 182_11_B22_34 | hypothetical protein A458_07350 | YP_006457139.1 | 30179 | 30391 | #N/A | #N/A |
| 182_11_B22_35 | hypothetical protein PSTAB_1320 | YP_004713690.1 | 30436 | 30621 | #N/A | #N/A |
| 182_11_B22_36 | alginate biosynthesis transcriptional activa | YP_004713688.1 | 30849 | 31166 | #N/A | #N/A |
| 182_11_B22_37 | oxaloacetate decarboxylase subunit beta | YP_005938059.1 | 31468 | 32604 | #N/A | #N/A |
| 182_11_B22_38 | pyruvate carboxylase subunit B | YP_004713686.1 | 32615 | 34393 | #N/A | #N/A |
| 182_11_B22_39 | sodium pump decarboxylase, gamma | YP_006457134.1 | 34416 | 34658 | #N/A | #N/A |
| | subunit | | | | | |
| 182_11_B22_40 | magnesium transporter | YP_004713684.1 | 34799 | 36235 | #N/A | #N/A |
| 182_11_B22_41 | hypothetical protein PST_1375 | YP_001171909.1 | 36572 | 37054 | #N/A | #N/A |
| 182_11_B22_42 | carbon storage regulator | YP_001171908.1 | 37591 | 37776 | #N/A | #N/A |
| 182_11_B22_43 | aspartate kinase | YP_006457130.1 | 37957 | 39195 | #N/A | #N/A |
| 182_11_B22_44 | alanyl-tRNA synthetase | YP_001171906.1 | 39275 | 40012 | #N/A | #N/A |
| 182_13_F13_1 | phage integrase family protein | EGM14140.1 | 3 | 2480 | #N/A | #N/A |
| 182_13_F13_2 | phage integrase family protein | EGM16032.1 | 2486 | 3868 | #N/A | #N/A |
| 182_13_F13_3 | oxygen-independent coproporphyrinogen III | YP_006458415. 1 | 4026 | 4151 | #N/A | Oxido |
| | ox | | | | | |
| 182_13_F13_4 | TetR family transcriptional regulator | YP_006458416.1 | 4183 | 4803 | #N/A | #N/A |
| 182_13_F13_5 | class V aminotransferase | YP_001173104.1 | 4879 | 6012 | #N/A | #N/A |
| 182_13_F13_6 | aromatic amino acid transport protein AroP1 | YP_006458418.1 | 6233 | 7627 | #N/A | #N/A |
| 182_13_F13_7 | hydrolase, TatD family | YP_006458419.1 | 7739 | 8524 | #N/A | #N/A |
| 182_13_F13_8 | type 4 fimbrial biogenesis protein PilZ | YP_006458420.1 | 8628 | 8984 | #N/A | Secretion |
| 182_13_F13_9 | DNA polymerase III subunit delta′ | YP_006458421.1 | 9016 | 10002 | #N/A | #N/A |
| 182_13_F13_10 | thymidylate kinase | YP_001173109.1 | 9995 | 10627 | #N/A | #N/A |
| 182_13_F13_11 | hypothetical protein PST_2618 | YP_001173110.1 | 10624 | 11694 | #N/A | #N/A |
| 182_13_F13_12 | 4-amino-4-deoxychorismate lyase | EHY78332.1 | 11691 | 12512 | #N/A | #N/A |
| 182_13_F13_13 | 3-oxoacyl-(acyl carrier protein) synthase II | EHY78333.1 | 12509 | 13753 | #N/A | #N/A |
| 182_13_F13_14 | acyl carrier protein | YP_001173113.1 | 13926 | 14162 | #N/A | #N/A |
| 182_13_F13_15 | 3-ketoacyl-ACP reductase | YP_001173114.1 | 14355 | 15098 | #N/A | #N/A |
| 182_13_F13_16 | malonyl-CoA- | YP_001173115.1 | 15113 | 16051 | #N/A | #N/A |
| 182_13_F13_17 | plsX gene product | YP_005939366.1 | 16115 | 17185 | #N/A | #N/A |
| 182_13_F13_18 | 50S ribosomal protein L32 | EHY78338.1 | 17189 | 17371 | #N/A | #N/A |
| 182_13_F13_19 | metal-binding protein | YP_006458431.1 | 17384 | 17911 | #N/A | #N/A |
| 182_13_F13_20 | Maf-like protein | EHY78340.1 | 18015 | 18593 | #N/A | #N/A |
| 182_13_F13_21 | signal peptide peptidase | EHY78341.1 | 18604 | 19587 | #N/A | #N/A |
| 182_13_F13_22 | HAD superfamily hydrolase | YP_006458434.1 | 19577 | 20263 | #N/A | #N/A |
| 182_13_F13_23 | ribosomal large subunit pseudouridine | YP_006458435.1 | 20256 | 21209 | #N/A | #N/A |
| | syntha | | | | | |
| 182_13_F13_24 | ribonuclease E | YP_006458436.1 | 21768 | 24965 | #N/A | #N/A |
| 182_13_F13_25 | UDP-N-acetylenolpyruvoylglucosamine | YP_006458437.1 | 25357 | 26376 | #N/A | #N/A |
| | reductas | | | | | |
| 182_13_F13_26 | protein-tyrosine-phosphatase | YP_005939375.1 | 26373 | 26537 | #N/A | #N/A |
| 182_16_E12_1 | putative secreted protein | ZP_10760484.1 | 2 | 382 | SEC | Secretion |
| 182_16_E12_2 | hypothetical protein | YP_001186721.1 | 476 | 1171 | #N/A | #N/A |
| 182_16_E12_3 | MACP | ZP_10706566.1 | 1168 | 2310 | #N/A | MACP |
| 182_16_E12_4 | AraC family transcriptional regulator | ZP_09709445.1 | 2468 | 3217 | #N/A | #N/A |
| 182_16_E12_5 | methyl-accepting chemotaxis sensory | YP_001186850.1 | 3254 | 4891 | TM | MACP |
| | transduc | | | | | |
| 182_16_E12_6 | putA gene product | NP_249473.1 | 5214 | 8381 | #N/A | #N/A |
| 182_16_E12_7 | hypothetical protein BN5_00960 | ZP_10760509.1 | 8682 | 10169 | #N/A | #N/A |
| 182_16_E12_8 | NADH:flavin oxidoreductase | ZP_10788615.1 | 10278 | 11519 | SEC | Oxido |
| 182_16_E12_9 | response regulator | YP_431980.1 | 11752 | 12867 | #N/A | #N/A |
| 182_16_E12_10 | Multidrug resistance protein | YP_004380949.1 | 12901 | 16056 | #N/A | MDES |
| 182_16_E12_11 | RND family efflux transporter MFP subunit | YP_001187176.1 | 16058 | 17143 | SEC | MDES |
| 182_16_E12_12 | HTH-type transcriptional regulator betI | ZP_10761345.1 | 17146 | 17805 | #N/A | #N/A |
| 182_16_E12_13 | conserved hypothethical protein, SAM- | CBJ39758.1 | 18244 | 19002 | #N/A | #N/A |
| | dependent m | | | | | |
| 182_16_E12_14 | FAD-dependent oxidoreductase | EJX16335.1 | 19089 | 20378 | #N/A | Oxido |
| 182_16_E12_15 | XRE family transcriptional regulator | ZP_10149413.1 | 20418 | 20975 | #N/A | #N/A |
| 182_16_E12_16 | glutamine synthetase | YP_001268868.1 | 21009 | 22361 | #N/A | #N/A |
| 182_16_E12_17 | MerR family transcriptional regulator | YP_932982.1 | 22412 | 22789 | #N/A | #N/A |
| 182_16_E12_18 | NADPH-dependent reductase | EKA19030.1 | 22844 | 23422 | #N/A | Oxido |
| 182_16_E12_19 | hypothetical protein PST_2845 | YP_001173333.1 | 23588 | 24127 | #N/A | #N/A |
| 182_16_E12_20 | hypothetical protein | YP_001188111.1 | 24085 | 24297 | #N/A | #N/A |
| 182_16_E12_21 | glycine/D-amino acid oxidase | ZP_10761703.1 | 24435 | 25718 | SEC | HPG |
| 182_16_E12_22 | threonyl-tRNA synthetase | YP_004380708.1 | 26204 | 28126 | #N/A | #N/A |
| 182_16_E12_23 | translation initiation factor IF-3 | YP_001748840.1 | 28144 | 28677 | #N/A | #N/A |
| 182_16_E12_24 | 50S ribosomal protein L35 | YP_004474812.1 | 28738 | 28932 | #N/A | #N/A |
| 182_16_E12_25 | rplT gene product | YP_001187472.1 | 28961 | 29317 | #N/A | #N/A |
| 182_16_E12_26 | pheS gene product | YP_001187473.1 | 29412 | 30428 | #N/A | #N/A |
| 182_16_E12_27 | phenylalanyl-tRNA synthetase subunit beta | YP_004380703.1 | 30471 | 32273 | #N/A | #N/A |
| 182_16_J11_1 | Alcohol dehydrogenase GroES domain | ZP_09686224.1 | 182 | 361 | #N/A | Oxido |
| | protein | | | | | |
| 182_16_J11_2 | aromatic hydrocarbon degradation outer | AAC03445.1 | 409 | 1773 | SEC | Secretion |
| | membrane protein | | | | | |
| 182_16_J11_3 | methyl-accepting chemotaxis | YP_933345.1 | 1897 | 3249 | SEC | PAS |
| | transducer/PAS protein | | | | | |
| 182_16_J11_4 | Glycosyl hydrolase, BNR repeat | ZP_01893293.1 | 3749 | 4765 | #N/A | #N/A |
| 182_16_J11_5 | RND superfamily exporter | YP_005887713.1 | 4776 | 7232 | TM | MDES |
| 182_16_J11_6 | cox2 cytochrome oxidase subunit | ZP_01893295.1 | 7277 | 9166 | SEC | Oxido |
| 182_16_J11_7 | hypothetical protein | YP_005887711.1 | 9185 | 10489 | SEC | Secretion |
| 182_16_J11_8 | MACP, PAS domain S-box | ZP_01893297.1 | 10588 | 12135 | #N/A | MACP |
| 182_16_J11_9 | malonate decarboxylase, alpha subunit | EHY77067.1 | 12240 | 13907 | #N/A | #N/A |
| 182_16_J11_10 | triphosphoribosyl-dephospho-CoA synthase | YP_004716471.1 | 13907 | 14782 | #N/A | #N/A |
| 182_16_J11_11 | malonate decarboxylase subunit delta | YP_006455802.1 | 14785 | 15084 | #N/A | #N/A |
| 182_16_J11_12 | mdcD gene product | YP_005940865.1 | 15077 | 15943 | #N/A | #N/A |
| 182_16_J11_13 | malonate decarboxylase, gamma subunit | EHY77071.1 | 15940 | 16725 | #N/A | #N/A |
| 182_16_J11_14 | phosphoribosyl-dephospho-CoA transferase | YP_004716467.1 | 16798 | 17415 | #N/A | #N/A |
| 182_16_J11_15 | malonyl CoA-acyl carrier protein | YP_005940862.1 | 17412 | 18338 | #N/A | #N/A |
| | transacylas | | | | | |
| 182_16_J11_16 | malonate transporter, MadL subunit | EHY77074.1 | 18463 | 18885 | #N/A | #N/A |
| 182_16_J11_17 | malonate transporter subunit MadM | YP_001174576.1 | 18891 | 19655 | #N/A | #N/A |
| 182_16_J11_18 | FAD-dependent oxidoreductase | YP_006455809.1 | 20092 | 21351 | SEC | Oxido |
| 182_16_J11_19 | LysR family transcriptional regulator | EHY77076.1 | 21665 | 22582 | #N/A | #N/A |
| 182_16_J11_20 | hypothetical protein A458_00600 | YP_006455811.1 | 22641 | 23282 | #N/A | #N/A |
| 182_16_J11_21 | RNA polymerase sigma factor | YP_006455812.1 | 23297 | 23848 | #N/A | #N/A |
| 182_16_J11_22 | hypothetical protein A458_00610 | YP_006455813.1 | 24041 | 24313 | #N/A | #N/A |
| 182_16_J11_23 | hypothetical protein PstZobell_06533 | EHY77080.1 | 24330 | 25157 | #N/A | #N/A |
| 182_16_J11_24 | hypothetical protein PstZobell_06538 | EHY77081.1 | 25154 | 25930 | #N/A | #N/A |
| 182_16_J11_25 | DoxX family protein | EHY77082.1 | 25942 | 26433 | #N/A | #N/A |
| 182_16_J11_26 | hypothetical protein A458_00630 | YP_006455817.1 | 26455 | 26661 | #N/A | #N/A |
| 182_16_J11_27 | lipase, class 3 | YP_006455818.1 | 26827 | 28341 | #N/A | #N/A |
| 182_16_J11_28 | lipoprotein | YP_001186317.1 | 28975 | 29718 | #N/A | #N/A |
| 182_16_J11_29 | hypothetical protein A458_00645 | YP_006455820.1 | 29723 | 30568 | #N/A | #N/A |
| 182_16_J11_30 | Rhs element Vgr protein, type VI secretion | YP_006455821.1 | 30565 | 32076 | #N/A | Secretion |
| | system Vgr family protein | | | | | |
| 182_17_09_1 | choline transport protein BetT | EHY79645.1 | 3 | 1040 | #N/A | #N/A |
| 182_17_09_2 | glycine betaine aldehyde dehydrogenase | YP_005938402.1 | 1127 | 2599 | #N/A | Oxido |
| 182_17_09_3 | choline dehydrogenase | YP_001172266.1 | 2614 | 4287 | #N/A | HPG |
| 182_17_09_4 | ribosomal protein S12 methylthiotransferase | YP_006458067.1 | 4389 | 5711 | #N/A | #N/A |
| 182_17_09_5 | YesN family response regulator | EIK53762.1 | 5872 | 6744 | #N/A | #N/A |
| 182_17_09_6 | Flp pilus assembly protein, pilin Flp | ZP_10704409.1 | 7093 | 7284 | #N/A | Secretion |
| 182_17_09_7 | Flp pilus assembly protein, protease CpaA | ZP_10599436.1 | 7291 | 7812 | SEC | Secretion |
| 182_17_09_8 | hypothetical protein PMI26_01591 | ZP_10673849.1 | 7825 | 9147 | #N/A | Secretion |
| 182_17_09_9 | Flp pilus assembly protein, RcpC family | ZP_10173383.1 | 9160 | 9969 | TM | Secretion |
| 182_17_09_10 | type II and III secretion system protein | EIK53757.1 | 10024 | 11538 | SEC | Secretion |
| 182_17_09_11 | hypothetical protein Y05_15635 | EIK53756.1 | 11554 | 11823 | #N/A | #N/A |
| 182_17_09_12 | Flp pilus assembly protein TadG | EIK53755.1 | 11834 | 13168 | SEC | Secretion |
| 182_17_09_13 | Flp pilus assembly protein TadG | EIK53754.1 | 13180 | 13650 | TM | Secretion |
| 182_17_09_14 | Flp pilus assembly protein TadG | ZP_10665569.1 | 13650 | 14153 | TM | Secretion |
| 182_17_09_15 | type II/IV secretion system ATPase TadZ | EIK53752.1 | 14147 | 15376 | #N/A | Secretion |
| 182_17_09_16 | type II/IV secretion system protein | ZP_10614051.1 | 15366 | 16787 | #N/A | Secretion |
| 182_17_09_17 | type II secretion system protein F | ZP_10639299.1 | 16784 | 17770 | #N/A | Secretion |
| 182_17_09_18 | type II secretion system protein; membrane | YP_004355493.1 | 17781 | 18749 | #N/A | #N/A |
| | P | | | | | |
| 182_17_09_19 | TPR repeat protein | EIK53748.1 | 18751 | 19794 | #N/A | #N/A |
| 182_17_09_20 | O-antigen acetylase | YP_005938405.1 | 19807 | 20877 | #N/A | #N/A |
| 182_17_09_21 | glycosyl transferase family protein | YP_005938406.1 | 20900 | 21973 | #N/A | #N/A |
| 182_17_09_22 | hypothetical protein PSTAB_1644 | YP_004714014.1 | 21985 | 22179 | #N/A | #N/A |
| 182_17_09_23 | hypothetical protein PSTAB_1645 | YP_004714015.1 | 22219 | 22557 | #N/A | #N/A |
| 182_17_09_24 | glycoside hydrolase family protein | YP_001172271.1 | 22605 | 23807 | #N/A | #N/A |
| 182_17_09_25 | hypothetical protein PST_1752 | YP_001172272.1 | 23864 | 24910 | #N/A | #N/A |
| 182_17_09_26 | glycoside hydrolase family protein | YP_001172273.1 | 24965 | 26071 | #N/A | #N/A |
| 182_17_09_27 | hypothetical protein PstZobell_19633 | EHY79634.1 | 26277 | 27590 | #N/A | #N/A |
| 182_17_09_28 | hypothetical protein PST_1755 | YP_001172275.1 | 27616 | 28866 | #N/A | #N/A |
| 182_17_09_29 | glycosyl transferase, group 1 family protein | YP_006458056.1 | 28808 | 30172 | #N/A | #N/A |
| 182_17_09_30 | transcriptional activator RfaH | EHY79631.1 | 30180 | 30689 | #N/A | #N/A |
| 182_17_09_31 | hypothetical protein | YP_005938416.1 | 30744 | 31487 | #N/A | #N/A |
| 182_17_09_32 | tyrosine-protein kinase | YP_001172279.1 | 31510 | 33723 | #N/A | #N/A |
| 182_17_09_33 | glycosyl transferase family protein | YP_001172280.1 | 33773 | 34705 | #N/A | #N/A |
| 182_17_09_34 | polysaccharide biosynthesis protein | YP_006458051.1 | 34674 | 35285 | #N/A | #N/A |
| 182_35_020_1 | type 4 prepilin peptidase PilD | YP_004713329.1 | 1 | 165 | TM | Secretion |
| 182_35_020_2 | type II secretory pathway, component | YP_006458941.1 | 169 | 1389 | #N/A | Secretion |
| 182_35_020_3 | type IV-A pilus assembly ATPase PilB | YP_006458942.1 | 1392 | 3095 | #N/A | Secretion |
| 182_35_020_4 | Tfp structural protein | YP_004378671.1 | 3460 | 3645 | SEC | Secretion |
| 182_35_020_6 | hypothetical protein | YP_004378672.1 | 4854 | 5261 | #N/A | #N/A |
| 182_35_020_7 | hypothetical protein | YP_004378673.1 | 5262 | 6104 | #N/A | #N/A |
| 182_35_020_8 | putative ABC transporter ATP-binding | YP_004378674.1 | 6101 | 6997 | #N/A | MDES |
| | protein | | | | | |
| 182_35_020_9 | bifunctional sulfate adenylyltransferase | EHY76696.1 | 7083 | 8621 | #N/A | #N/A |
| | subunit | | | | | |
| 182_35_020_10 | sulfate adenylyltransferase subunit 2 | YP_001171589.1 | 8992 | 9909 | #N/A | #N/A |
| 182_35_020_11 | dinuclear metal center protein, putative | YP_006458951.1 | 10095 | 10853 | #N/A | HPG |
| | hydrolase-oxidas | | | | | |
| 182_35_020_12 | 2-alkenal reductase | EHY76699.1 | 11014 | 12171 | TM | Oxido |
| 182_35_020_13 | histidinol-phosphate aminotransferase | EHY76700.1 | 12267 | 13313 | #N/A | #N/A |
| 182_35_020_14 | bifunctional histidinal dehydrogenase/ histi | YP_006458954.1 | 13410 | 14720 | #N/A | #N/A |
| 182_35_020_15 | ATP phosphoribosyltransferase catalytic | YP_006458955.1 | 14890 | 15522 | #N/A | #N/A |
| | subu | | | | | |
| 182_35_020_16 | UDP-N-acetylglucosamine 1- | EHY76703.1 | 15758 | 17023 | #N/A | #N/A |
| | carboxyvinyltransferase | | | | | |
| 182_35_020_17 | toluene-tolerance protein | EHY76704.1 | 17127 | 17366 | #N/A | #N/A |
| 182_35_020_18 | hypothetical protein PST_1042 | YP_001171581.1 | 17466 | 17957 | #N/A | #N/A |
| 182_35_020_19 | toluene-tolerance protein | YP_004713314.1 | 17971 | 18285 | #N/A | #N/A |
| 182_35_020_20 | toluene-tolerance protein | EHY76707.1 | 18278 | 18925 | #N/A | #N/A |
| 182_35_020_21 | toluene tolerance ABC transporter | YP_001171578.1 | 18937 | 19395 | #N/A | #N/A |
| | periplasmi | | | | | |
| 182_35_020_22 | toluene tolerance ABC efflux transporter, pe | YP_001171577.1 | 19395 | 20192 | #N/A | #N/A |
| 182_35_020_23 | toluene tolerance ABC efflux transporter, | YP_001171576.1 | 20185 | 21000 | #N/A | #N/A |
| | AT | | | | | |
| 182_35_020_24 | hypothetical protein A458_16580 | YP_006458964.1 | 21282 | 22256 | #N/A | #N/A |
| 182_35_020_25 | YrbI family phosphatase | YP_001171574.1 | 22256 | 22780 | #N/A | #N/A |
| 182_35_020_26 | hypothetical protein A458_16590 | YP_006458966.1 | 22789 | 23361 | #N/A | #N/A |
| 182_35_020_27 | OstA family protein | YP_006458967.1 | 23348 | 23893 | #N/A | #N/A |
| 182_35_020_28 | hypothetical protein | CAA11111.1 | 23893 | 24618 | #N/A | #N/A |
| 182_35_020_29 | sigma factor sigma-54 | CAA11112.1 | 24764 | 26272 | #N/A | #N/A |
| 182_35_020_30 | sigma54 modulation protein | YP_006458970.1 | 26347 | 26655 | #N/A | #N/A |
| 182_35_020_31 | phosphotransferase enzyme HA | YP_006458971.1 | 26663 | 27127 | #N/A | #N/A |
| 182_35_020_32 | glmZ(sRNA)-inactivating NTPase | YP_006458972.1 | 27143 | 28000 | #N/A | #N/A |
| 182_35_020_33 | phosphotransferase system, phosphocarrier | YP_006458973.1 | 28015 | 28287 | #N/A | #N/A |
| | pr | | | | | |
| 182_35_020_34 | PmbA protein | YP_006458974.1 | 28340 | 29686 | #N/A | #N/A |
| 182_35_020_35 | hypothetical protein A458_16635 | YP_006458975.1 | 29799 | 30320 | #N/A | #N/A |
| 182_35_020_36 | peptidase U62, modulator of DNA gyrase | EHY76723.1 | 30396 | 31838 | #N/A | #N/A |
| 182_35_020_37 | carbon-nitrogen hydrolase family protein | YP_006458977.1 | 31841 | 32551 | #N/A | #N/A |
| 182_42_K21_1 | acyl-CoA dehydrogenase domain-containing | EHY77519.1 | 2 | 1057 | #N/A | #N/A |
| | protein | | | | | |
| 182_42_K21_2 | peptide methionine sulfoxide reductase | YP_006456115.1 | 1472 | 2119 | SEC | Oxido |
| 182_42_K21_3 | PAS/PAC and GAF sensor-containing | YP_006456116.1 | 2231 | 4903 | #N/A | PAS |
| 182_42_K21_4 | TPR repeat-containing protein | YP_001174319.1 | 5014 | 5541 | #N/A | #N/A |
| 182_42_K21_5 | dihydrolipoamide acetyltransferase | YP_004716174.1 | 5650 | 7656 | #N/A | #N/A |
| 182_42_K21_6 | pyruvate dehydrogenase subunit E1 | YP_004716173.1 | 7681 | 10326 | #N/A | #N/A |
| 182_42_K21_7 | glutamate-ammonia-ligase | YP_004716172.1 | 10593 | 13538 | #N/A | #N/A |
| | adenylyltransferase | | | | | |
| 182_42_K21_8 | branched-chain amino acid aminotransferase | YP_004716171.1 | 13589 | 14512 | #N/A | #N/A |
| 182_42_K21_9 | heptosyltransferase II | YP_004716170.1 | 14591 | 15625 | #N/A | #N/A |
| 182_42_K21_10 | lipopolysaccharide heptosyltransferase I | YP_004716169.1 | 15626 | 16627 | #N/A | #N/A |
| 182_42_K21_11 | waaG gene product | YP_005940538.1 | 16627 | 17748 | #N/A | #N/A |
| 182_42_K21_12 | lipopolysaccharide core biosynthesis protein | YP_004716167.1 | 17792 | 18598 | #N/A | #N/A |
| 182_42_K21_13 | lipopolysaccharide kinase | YP_005940536.1 | 18598 | 19332 | #N/A | #N/A |
| 182_42_K21_14 | lipopolysaccharide kinase | YP_005940535.1 | 19329 | 20072 | #N/A | #N/A |
| 182_42_K21_15 | serine/threonine protein kinase | YP_006456128.1 | 20072 | 21517 | #N/A | #N/A |
| 182_42_K21_16 | carbamoyl transferase | YP_005940533.1 | 21530 | 23284 | #N/A | #N/A |
| 182_42_K21_17 | group 1 glycosyl transferase | YP_005940532.1 | 23271 | 24383 | #N/A | #N/A |
| 182_42_K21_18 | putative acetyltransferase | YP_004378444.1 | 24461 | 25189 | #N/A | #N/A |
| 182_42_K21_19 | hypothetical protein | YP_004378445.1 | 25186 | 26145 | #N/A | #N/A |
| 182_42_K21_20 | hypothetical protein PSTAB_3787 | YP_004716157.1 | 26148 | 27020 | #N/A | #N/A |
| 182_42_K21_21 | hypothetical protein PSTAB_3786 | YP_004716156.1 | 27024 | 27782 | #N/A | #N/A |
| 182_42_K21_22 | hypothetical protein PSTAB_3785 | YP_004716155.1 | 27779 | 28930 | #N/A | #N/A |
| 182_42_K21_23 | Ttg8 | YP_004716154.1 | 28955 | 29572 | #N/A | #N/A |
| 182_42_K21_24 | lipid A ABC exporter, fused ATPase and | YP_004716153.1 | 29687 | 31495 | #N/A | #N/A |
| | inner | | | | | |
| 182_42_K21_25 | Mig-14 family protein | YP_006456137.1 | 31495 | 32391 | #N/A | #N/A |
| 182_42_K21_26 | LmbE family protein | YP_006456138.1 | 32395 | 33789 | #N/A | #N/A |
| 182_42_K21_27 | rfaE gene product | YP_005940529.1 | 33870 | 35291 | #N/A | #N/A |
| 182_42_K21_28 | hypothetical protein PST_3822 | YP_001174287.1 | 35326 | 36213 | #N/A | #N/A |
| 182_42_K21_29 | putative oxidoreductase, aryl-alcohol | YP_004716148.1 | 36299 | 37108 | #N/A | HPG |
| | dehydro | | | | | |
| 182_42_K21_30 | oxidoreductase, FAD-binding protein | EHY79403.1 | 37105 | 38280 | #N/A | Oxido |
| 182_42_K21_31 | multidrug efflux SMR transporter | YP_005940525.1 | 38273 | 38671 | TM | MDES |
| 182_42_K21_32 | 3-deoxy-D-manno-octulosonic-acid | YP_004716145.1 | 38766 | 39464 | #N/A | #N/A |
| | transferase | | | | | |
| 183_01_D18_1 | Alcohol dehydrogenase zinc-binding domain | YP_146887.1 | 1 | 813 | #N/A | Oxido |
| 183_01_D18_2 | acyl-CoA dehydrogenase | BAL25048.1 | 859 | 2646 | #N/A | #N/A |
| 183_01_D18_3 | nitroreductase | NP_889788.1 | 2673 | 3263 | #N/A | #N/A |
| 183_01_D18_4 | resorcinol hydroxylase small subunit | ABK58619.1 | 3476 | 4375 | #N/A | #N/A |
| 183_01_D18_5 | 6-phosphogluconate dehydrogenase NAD- | ZP_05878811.1 | 4438 | 5319 | #N/A | #N/A |
| | binding | | | | | |
| 183_01_D18_7 | Enoyl-CoA hydratase/isomerase | YP_004304180.1 | 5769 | 6632 | #N/A | #N/A |
| 183_01_D18_8 | aldehyde dehydrogenase | BAL55144.1 | 6692 | 8170 | #N/A | #N/A |
| 183_01_D18_9 | Dehydrogenase E1 component superfamily | ZP_05095591.1 | 8195 | 9181 | #N/A | #N/A |
| | protei | | | | | |
| 183_01_D18_10 | Transketolase, C-terminal domain protein | ZP_05095594.1 | 9196 | 10179 | #N/A | #N/A |
| 183_0l_D18_11 | 2-oxo acid dehydrogenases acyltransferase | ZP_05095650.1 | 10189 | 11457 | #N/A | #N/A |
| | (ca | | | | | |
| 183_01_D18_12 | dihydrolipoyl dehydrogenase | ZP_05095656.1 | 11466 | 12878 | #N/A | #N/A |
| 183_01_D18_13 | acyl-CoA hydrolase | YP_005026581.1 | 12892 | 13329 | #N/A | #N/A |
| 183_01_D18_14 | Putative bifunctional protein 3-hydroxyacyl- | ZP_08505254.1 | 13364 | 15748 | #N/A | #N/A |
| | C | | | | | |
| 183_01_D18_15 | acetyl-CoA acetyltransferase | BAL26541.1 | 15758 | 16954 | #N/A | #N/A |
| 183_01_D18_16 | thioesterase | YP_568644.1 | 16959 | 17375 | #N/A | #N/A |
| 183_01_D18_17 | transcriptional regulator, TetR family | EJO59715.1 | 17632 | 18285 | #N/A | #N/A |
| 183_01_D18_18 | hypothetical protein | YP_003607667.1 | 18317 | 19375 | #N/A | #N/A |
| 183_01_D18_19 | Protein of unknown function (DUF1329) | ZP_10701087.1 | 19388 | 20749 | #N/A | #N/A |
| 183_01_D18_20 | putative photosystem II stability/assembly fa | ZP_10606344.1 | 20878 | 21975 | #N/A | #N/A |
| 183_01_D18_21 | putative RND superfamily exporter | YP_003607682.1 | 21975 | 24422 | TM | MDES |
| 183_01_D18_22 | hypothetical protein AZKH_p0596 | BAL27479.1 | 24441 | 25466 | #N/A | #N/A |
| 183_01_D18_23 | major facilitator transporter | YP_001811493.1 | 25543 | 26712 | TM | MDES |
| 183_01_D18_24 | integrase catalytic region protein | YP_004127365.1 | 27088 | 27576 | #N/A | #N/A |
| 183_01_D18_25 | putative type III effector Hop protein | YP_002354618.1 | 27956 | 28321 | SEC | Secretion |
| 183_01_D18_26 | Integrating conjugative element protein | YP_986434.1 | 28318 | 28557 | #N/A | #N/A |
| 183_01_D18_27 | integrating conjugative element | YP_002354616.1 | 28580 | 28948 | #N/A | #N/A |
| 183_01_D18_28 | conjugative transfer region protein | YP_004713584.1 | 28961 | 29371 | #N/A | #N/A |
| 183_01_D18_29 | integrating conjugative element protein | YP_002354614.1 | 29451 | 30143 | #N/A | #N/A |
| 183_01_D18_30 | putative secreted protein | YP_001021581.1 | 30140 | 31054 | #N/A | #N/A |
| 183_01_D18_31 | integrating conjugative element protein | YP_004387701.1 | 31044 | 32477 | #N/A | #N/A |
| 183_01_D18_32 | Conjugative transfer region lipoprotein | YP_001021583.1 | 32458 | 32892 | #N/A | #N/A |
| 183_01_D18_33 | conjugative transfer ATPase | EKA41066.1 | 32892 | 34694 | #N/A | #N/A |
| 183_12_O16_1 | RND efflux transporter permease | YP_002890274.1 | 2 | 928 | TM | MDES |
| 183_12_O16_2 | RND family efflux transporter MFP subunit | YP_002890275.1 | 943 | 2001 | SEC | MDES |
| 183_12_O16_3 | cobalamin (vitamin B12) biosynthesis CbiX | YP_002890276.1 | 2082 | 2468 | #N/A | #N/A |
| | pr | | | | | |
| 183_12_O16_4 | UBA/THIF-type NAD/FAD-binding protein | YP_002890277.1 | 2548 | 3348 | #N/A | #N/A |
| 183_12_O16_5 | Zn-dependent hydrolase | YP_002890278.1 | 3356 | 4318 | #N/A | #N/A |
| 183_12_O16_6 | single-strand binding protein | YP_002890279.1 | 4506 | 5009 | #N/A | #N/A |
| 183_12_O16_7 | major facilitator superfamily protein | YP_002890280.1 | 5067 | 6338 | TM | MDES |
| 183_12_O16_8 | excinuclease ABC subunit A | BAL26253.1 | 6432 | 9317 | #N/A | #N/A |
| 183_12_O16_9 | UDP-glucose 4-epimerase | YP_002890282.1 | 9278 | 10324 | #N/A | #N/A |
| 183_12_O16_10 | 50S ribosomal protein L17 | YP_002890283.1 | 10425 | 10823 | #N/A | #N/A |
| 183_12_O16_11 | DNA-directed RNA polymerase subunit | YP_002890284.1 | 10849 | 11829 | #N/A | #N/A |
| | alpha | | | | | |
| 183_12_O16_12 | 30S ribosomal protein S4 | YP_002890285.1 | 11869 | 12498 | #N/A | #N/A |
| 183_12_O16_13 | 30S ribosomal protein S11 | YP_002890286.1 | 12513 | 12902 | #N/A | #N/A |
| 183_12_O16_14 | rpsM gene product | YP_934896.1 | 12915 | 13277 | #N/A | #N/A |
| 183_12_O16_15 | 50S ribosomal protein L36 | YP_002890288.1 | 13331 | 13444 | #N/A | #N/A |
| 183_12_O16_16 | translation initiation factor 1F-1 | YP_002890289.1 | 13470 | 13688 | #N/A | #N/A |
| 183_12_O16_17 | preprotein translocase subunit SecY | YP_002890290.1 | 13693 | 15018 | #N/A | #N/A |
| 183_12_O16_18 | 50S ribosomal protein L15 | YP_002890291.1 | 15034 | 15471 | #N/A | #N/A |
| 183_12_O16_19 | 50S ribosomal protein L30 | YP_002890292.1 | 15473 | 15655 | #N/A | #N/A |
| 183_12_O16_20 | 30S ribosomal protein S5 | YP_002890293.1 | 15659 | 16183 | #N/A | #N/A |
| 183_12_O16_21 | 50S ribosomal protein L18 | YP_002890294.1 | 16196 | 16549 | #N/A | #N/A |
| 183_12_O16_22 | rplF gene product | YP_934904.1 | 16561 | 17094 | #N/A | #N/A |
| 183_12_O16_23 | 30S ribosomal protein S8 | YP_002890296.1 | 17105 | 17500 | #N/A | #N/A |
| 183_12_O16_24 | 30S ribosomal protein S14 | YP_002890297.1 | 17514 | 17819 | #N/A | #N/A |
| 183_12_O16_25 | 50S ribosomal protein L5 | YP_002890298.1 | 17827 | 18366 | #N/A | #N/A |
| 183_12_O16_26 | 50S ribosomal protein L24 | YP_002890299.1 | 18376 | 18693 | #N/A | #N/A |
| 183_12_O16_27 | 50S ribosomal protein L14 | YP_002890300.1 | 18705 | 19073 | #N/A | #N/A |
| 183_12_O16_28 | 30S ribosomal protein S17 | YP_002890301.1 | 19227 | 19496 | #N/A | #N/A |
| 183_12_O16_29 | 50S ribosomal protein L29 | YP_002890302.1 | 19493 | 19687 | #N/A | #N/A |
| 183_12_O16_30 | 50S ribosomal protein L16 | YP_002890303.1 | 19690 | 20106 | #N/A | #N/A |
| 183_12_O16_31 | 30S ribosomal protein S3 | YP_002890304.1 | 20106 | 20876 | #N/A | #N/A |
| 183_12_O16_32 | 50S ribosomal protein L22 | BAL26278.1 | 20886 | 21215 | #N/A | #N/A |
| 183_12_O16_33 | 30S ribosomal protein S19 | YP_002890306.1 | 21230 | 21505 | #N/A | #N/A |
| 183_12_O16_34 | 50S ribosomal protein L2 | YP_002890307.1 | 21516 | 22343 | #N/A | #N/A |
| 183_12_O16_35 | 50S ribosomal protein L23 | YP_002890308.1 | 22350 | 22655 | #N/A | #N/A |
| 183_12_O16_36 | 50S ribosomal protein L4 | YP_002890309.1 | 22652 | 23272 | #N/A | #N/A |
| 183_12_O16_37 | 50S ribosomal protein L3 | YP_002890310.1 | 23283 | 23921 | #N/A | #N/A |
| 183_12_O16_38 | 30S ribosomal protein S10 | YP_002890311.1 | 24034 | 24345 | #N/A | #N/A |
| 183_12_O16_39 | elongation factor Tu | YP_002890312.1 | 24432 | 25622 | #N/A | #N/A |
| 183_12_O16_40 | elongation factor G | YP_002890313.1 | 25673 | 27772 | #N/A | #N/A |
| 183_12_O16_41 | rpsG gene product | YP_934923.1 | 27880 | 28347 | #N/A | #N/A |
| 183_12_O16_42 | 30S ribosomal protein S12 | YP_002890315.1 | 28381 | 28758 | #N/A | #N/A |
| 183_12_O16_43 | DNA-directed RNA polymerase subunit | YP_002890316.1 | 28893 | 32855 | #N/A | #N/A |
| | beta′ | | | | | |
| 183_21_D14_1 | HAD-superfamily hydrolase, subfamily IA, | ZP_04763958.1 | 2 | 862 | #N/A | #N/A |
| | vari | | | | | |
| 183_21_D14_2 | integral membrane protein | ZP_09330623.1 | 908 | 1774 | #N/A | #N/A |
| 183_21_D14_3 | conserved hypothetical protein | ZP_04763956.1 | 1771 | 1983 | #N/A | #N/A |
| 183_21_D14_4 | glutathione S-transferase-like protein | ZP_08405276.1 | 2033 | 2692 | SEC | #N/A |
| 183_21_D14_5 | RND efflux system outer membrane | ZP_10392050.1 | 2799 | 4214 | SEC | MDES |
| | lipoprotein | | | | | |
| 183_21_D14_6 | RND family efflux transporter MFP subunit | ZP_09331408.1 | 4318 | 5673 | TM | MDES |
| 183_21_D14_7 | ABC transporter related protein | ZP_04764465.1 | 5670 | 6419 | #N/A | #N/A |
| 183_21_D14_8 | ABC-type antimicrobial peptide transport | ZP_10392053.1 | 6419 | 7621 | #N/A | #N/A |
| | syst | | | | | |
| 183_21_D14_9 | response regulator receiver modulated | ZP_09330508.1 | 7712 | 8917 | #N/A | #N/A |
| | diguany PAS | | | | | |
| 183_21_D14_10 | heat shock protein Hsp20 | ZP_04764463.1 | 9126 | 9512 | #N/A | #N/A |
| 183_21_D14_11 | abc-type branched-chain amino acid | ZP_08946973.1 | 9607 | 10953 | #N/A | #N/A |
| | transporte | | | | | |
| 183_21_D14_12 | alpha/beta hydrolase fold protein | ZP_09752241.1 | 11016 | 11927 | #N/A | #N/A |
| 183_21_D14_13 | transposase IS116/IS110/IS902 family | YP_985458.1 | 12278 | 13240 | #N/A | #N/A |
| | protein | | | | | |
| 183_21_D14_14 | alpha/beta hydrolase family protein | ZP_09752242.1 | 13267 | 14127 | #N/A | #N/A |
| 183_21_D14_15 | acyltransferase, WS/DGAT/MGAT | ZP_09752243.1 | 14137 | 15723 | #N/A | #N/A |
| 183_21_D14_16 | PAS/PAC sensor-containing diguanylate | YP_003674696.1 | 15903 | 16730 | #N/A | PAS |
| 183_21_D14_17 | lytic murein transglycosylase B | ZP_04764460.1 | 16941 | 18035 | #N/A | #N/A |
| 183_21_D14_18 | transglutaminase-like enzyme, predicted | ZP_10392071.1 | 18032 | 20077 | #N/A | #N/A |
| | cyste | | | | | |
| 183_21_D14_19 | hypothetical protein AradN_05929 | ZP_08946985.1 | 20160 | 21170 | #N/A | #N/A |
| 183_21_D14_20 | ATPase | ZP_08946986.1 | 21191 | 22111 | #N/A | #N/A |
| 183_21_D14_21 | histone deacetylase superfamily protein | ZP_08946987.1 | 22154 | 23107 | #N/A | #N/A |
| 183_2l_D14_22 | enoyl-CoA hydratase/carnithine racemase | ZP_10392075.1 | 23126 | 23911 | #N/A | #N/A |
| 183_21_D14_23 | mechanosensitive ion channel protein MscS | ZP_09330497.1 | 23911 | 25233 | #N/A | #N/A |
| 183_21_D14_24 | electron transfer flavoprotein subunit alpha | YP_001563413.1 | 25488 | 26237 | #N/A | Oxido |
| 183_21_D14_25 | electron transfer flavoprotein subunit alpha | YP_004490435.1 | 26390 | 27322 | #N/A | Oxido |
| 183_21_D14_26 | acyl-CoA dehydrogenase domain protein | ZP_04763262.1 | 27499 | 29289 | #N/A | #N/A |
| 183_21_D14_27 | 2-nitropropane dioxygenase | YP_987024.1 | 29416 | 30372 | #N/A | #N/A |
| 183_21_D14_28 | acetate-CoA ligase | ZP_08947242.1 | 30482 | 32476 | #N/A | #N/A |
| 183_21_D14_29 | cytochrome c class I | ZP_09330598.1 | 33163 | 33468 | SEC | Oxido |
| 183_21_D14_30 | conserved hypothetical protein | ZP_04761189.1 | 33570 | 33812 | #N/A | #N/A |
| 183_21_D14_31 | dihydroxy-acid dehydratase | ZP_04761188.1 | 34036 | 35892 | #N/A | #N/A |
| 183_21_D14_32 | virulence-associated protein C | YP_002019545.1 | 36115 | 36525 | #N/A | #N/A |
| 183_21_D14_33 | Virulence-associated protein | YP_001791982.1 | 36525 | 36758 | #N/A | #N/A |
| 183_21_D14_34 | type III restriction protein res subunit | ZP_08951097.1 | 36895 | 38076 | #N/A | #N/A |
| 183_24_C18_1 | hypothetical protein | NP_715697.1 | 2 | 685 | #N/A | #N/A |
| 183_24_C18_3 | hypothetical protein PMI14_02990 | ZP_10390311.1 | 836 | 1678 | #N/A | #N/A |
| 183_24_C18_4 | lactoylglutathione lyase | ZP_09329249.1 | 1777 | 2247 | #N/A | #N/A |
| 183_24_C18_5 | hypothetical protein MEA186_14922 | ZP_09088162.1 | 2432 | 2938 | #N/A | #N/A |
| 183_24_C18_6 | biotin synthase | ZP_08946418.1 | 3443 | 4534 | #N/A | #N/A |
| 183_24_C18_7 | response regulator receiver modulated metal | ZP_09329251.1 | 4568 | 5758 | #N/A | MACP |
| | d | | | | | |
| 183_24_C18_8 | hypothetical protein AradN_03058 | ZP_08946416.1 | 5802 | 8843 | SEC | PAS |
| 183_24_C18_9 | Molybdopterin-binding protein KYG_10890 | ZP_09329253.1 | 8862 | 9398 | SEC | Oxido |
| 183_24_C18_10 | alkylhydroperoxidase AhpD | ZP_09329254.1 | 9640 | 9993 | #N/A | Oxido |
| 183_24_C18_11 | putative transmembrane protein | ZP_04763118.1 | 10024 | 10215 | #N/A | #N/A |
| 183_24_C18_12 | metallo-beta-lactamase superfamily protein | ZP_09329257.1 | 10313 | 11173 | #N/A | #N/A |
| 183_24_C18_13 | ArsR family regulatory protein | ZP_08950487.1 | 11279 | 11635 | #N/A | #N/A |
| 183_24_C18_14 | hypothetical protein KYG_10920 | ZP_09329259.1 | 11638 | 12069 | #N/A | #N/A |
| 183_24_C18_15 | hypothetical protein KYG_10925 | ZP_09329260.1 | 12117 | 12557 | #N/A | #N/A |
| 183_24_C18_16 | hypothetical protein KYG_10930 | ZP_09329261.1 | 12559 | 12903 | #N/A | #N/A |
| 183_24_C18_17 | site-specific recombinase XerD | ZP_10389702.1 | 12934 | 14007 | #N/A | #N/A |
| 183_24_C18_18 | KfrA domain-containing protein DNA- | YP_004234961.1 | 14125 | 15120 | #N/A | #N/A |
| | binding d | | | | | |
| 183_24_C18_19 | Diguanylate cyclase/phosphodiesterase | ZP_10137153.1 | 15424 | 17808 | #N/A | #N/A |
| | domain | | | | | |
| 183_24_C18_20 | short-chain dehydrogenase/reductase SDR | ZP_08946404.1 | 17987 | 18691 | #N/A | #N/A |
| 183_24_C18_21 | mate efflux family protein | ZP_08946403.1 | 18782 | 20188 | #N/A | #N/A |
| 183_24_C18_22 | MaoC-like protein dehydratase | ZP_08946402.1 | 20185 | 20694 | #N/A | #N/A |
| 183_24_C18_23 | major facilitator transporter | ZP_08946401.1 | 20737 | 22014 | TM | MDES |
| 183_24_C18_24 | MarR family transcriptional regulator | ZP_08946400.1 | 22004 | 22504 | #N/A | #N/A |
| 183_24_C18_25 | thioesterase superfamily protein | ZP_04762462.1 | 22578 | 23129 | #N/A | #N/A |
| 183_24_C18_26 | lactoylglutathione lyase | ZP_08946398.1 | 23126 | 23539 | #N/A | #N/A |
| 183_24_C18_27 | nicotinamidase-like amidase | ZP_10390427.1 | 23665 | 24270 | #N/A | #N/A |
| 183_24_C18_28 | NLP/P60 protein | ZP_04763442.1 | 24545 | 25135 | #N/A | #N/A |
| 183_24_C18_29 | hypothetical protein KYG_21454 | ZP_09331318.1 | 25183 | 25566 | #N/A | #N/A |
| 183_24_C18_30 | putative membrane protein | ZP_10390431.1 | 25776 | 26027 | #N/A | #N/A |
| 183_26_G23_1 | cyanophycin synthetase | ZP_10393306.1 | 1 | 1599 | #N/A | #N/A |
| 183_26_G23_2 | CreA family protein | ZP_08950440.1 | 1596 | 2084 | #N/A | #N/A |
| 183_26_G23_3 | DSBA oxidoreductase | ZP_08950443.1 | 2175 | 2822 | #N/A | Oxido |
| 183_26_G23_4 | hypothetical protein KYG_20310 | ZP_09331096.1 | 3134 | 3418 | #N/A | #N/A |
| 183_26_G23_5 | hypothetical protein PMI14_06112 | ZP_10393302.1 | 3512 | 4894 | #N/A | #N/A |
| 183_26_G23_6 | glucose-6-phosphate isomerase | ZP_09330015.1 | 4943 | 6499 | #N/A | #N/A |
| 183_26_G23_7 | 3-oxoacyl-ACP synthase | YP_005436677.1 | 6649 | 7767 | #N/A | #N/A |
| 183_26_G23_8 | transaldolase | ZP_04763152.1 | 7847 | 8794 | #N/A | #N/A |
| 183_26_G23_9 | RpiR family transcriptional regulator | ZP_08948106.1 | 8925 | 9770 | #N/A | #N/A |
| 183_26_G23_10 | PEBP family protein | ZP_04762018.1 | 9877 | 10371 | #N/A | #N/A |
| 183_26_G23_11 | 5′-nucleotidase | YP_972337.1 | 10422 | 12326 | #N/A | #N/A |
| 183_26_G23_12 | oligopeptide/dipeptide ABC transporter | ZP_08948108.1 | 12514 | 13518 | #N/A | #N/A |
| | ATPase | | | | | |
| 183_26_G23_13 | oligopeptide/dipeptide ABC transporter | ZP_09330000.1 | 13515 | 14495 | #N/A | #N/A |
| | ATPase | | | | | |
| 183_26_G23_14 | binding-protein-dependent transport systems | ZP_09329999.1 | 14669 | 15580 | #N/A | #N/A |
| | i | | | | | |
| 183_26_G23_15 | amidohydrolase | ZP_08948112.1 | 15598 | 16815 | #N/A | #N/A |
| 183_26_G23_16 | binding-protein-dependent transport systems | YP_002552144.1 | 16817 | 17797 | #N/A | #N/A |
| 183_26_G23_17 | family 5 extracellular solute-binding protein | ZP_09329995.1 | 17928 | 19508 | #N/A | #N/A |
| 183_26_G23_18 | porin | ZP_09329994.1 | 19811 | 20767 | #N/A | #N/A |
| 183_26_G23_19 | ubiquinone biosynthesis protein COQ7 | ZP_10391126.1 | 21084 | 21701 | #N/A | #N/A |
| 183_26_G23_20 | OsmC family protein | YP_969302.1 | 21872 | 22321 | #N/A | #N/A |
| 183_26_G23_21 | threonine dehydratase | ZP_09329991.1 | 22634 | 24211 | #N/A | #N/A |
| 183_26_G23_22 | cobalamin synthase | ZP_08948119.1 | 24510 | 25328 | #N/A | #N/A |
| 183_26_G23_23 | phosphoglycerate mutase | YP_984998.1 | 25325 | 25939 | #N/A | #N/A |
| 183_26_G23_24 | methyl-accepting chemotaxis sensory | ZP_09328098.1 | 25932 | 27578 | TM | MACP |
| | transduce | | | | | |
| 183_26_G23_25 | thiamine biosynthesis protein ThiC | ZP_04763400.1 | 27764 | 29659 | #N/A | #N/A |
| 183_26_G23_26 | hypothetical protein PMI12_02416 | ZP_10568386.1 | 29919 | 30095 | #N/A | #N/A |
| 183_26_G23_27 | udp-3-0-acyl n-acetylglucosamine | YP_004128296.1 | 30113 | 31036 | #N/A | #N/A |
| | deacetylase | | | | | |
| 183_26_G23_28 | cell division protein FtsZ | ZP_04763397.1 | 31147 | 32382 | #N/A | #N/A |
| 183_26_G23_29 | cell division protein FtsA | ZP_04763396.1 | 32543 | 33772 | #N/A | #N/A |
| 183_26_G23_30 | polypeptide-transport-associated domain- | ZP_08948139.1 | 33805 | 34593 | #N/A | #N/A |
| | conta | | | | | |
| 183_26_G23_31 | D-alanine/D-alanine ligase | ZP_04763394.1 | 34590 | 35576 | #N/A | #N/A |
| 183_26_G23_32 | UDP-N-acetylmuramate-L-alanine ligase | ZP_09328107.1 | 35576 | 37006 | #N/A | #N/A |
| 183_26_G23_33 | undecaprenyldiphospho- | ZP_09328108.1 | 37003 | 38088 | #N/A | #N/A |
| | muramoylpentapeptide be | | | | | |
| 183_52_O2_1 | hypothetical protein DelCs14_2697 | YP_004488064.1 | 1 | 5196 | #N/A | #N/A |
| 183_52_O2_3 | putative signal peptide protein | YP_345289.1 | 5430 | 5777 | #N/A | #N/A |
| 183_52_O2_4 | hsdR gene product | YP_005974822.1 | 6304 | 9102 | #N/A | #N/A |
| 183_52_O2_5 | hsdM gene product | YP_005974823.1 | 9115 | 10824 | #N/A | #N/A |
| 183_52_O2_6 | restriction modification system DNA | YP_001230860.1 | 10821 | 12416 | #N/A | #N/A |
| | specific | | | | | |
| 183_52_O2_7 | hypothetical protein AZA_26080 | ZP_08870323.1 | 12413 | 14287 | #N/A | #N/A |
| 183_52_O2_8 | hypothetical protein | YP_005974826.1 | 14287 | 15369 | #N/A | #N/A |
| 183_52_O2_9 | hypothetical protein ebA2393 | YP_158360.1 | 15453 | 16082 | #N/A | #N/A |
| 183_52_O2_10 | transcriptional regulator | YP_158359.1 | 16057 | 17226 | #N/A | #N/A |
| 183_52_O2_11 | hypothetical protein NCGM1179_3188 | GAA18352.1 | 17223 | 18596 | #N/A | #N/A |
| 183_52_O2_12 | hypothetical protein ebA2389 | YP_158357.1 | 18593 | 19159 | #N/A | #N/A |
| 183_52_O2_13 | ISxac2 transposase | YP_001172224.1 | 19305 | 19679 | #N/A | #N/A |
| 183_52_O2_14 | hypothetical protein PflSS101_1461 | EIK61223.1 | 19977 | 20654 | #N/A | #N/A |
| 183_52_O2_16 | Uncharacterized protein y4hO | ZP_10759747.1 | 20891 | 21238 | #N/A | #N/A |
| 183_52_O2_17 | prevent-host-death protein | ZP_04934490.1 | 21385 | 21630 | #N/A | #N/A |
| 183_52_O2_18 | plasmid stabilization system protein | ZP_04934489.1 | 21620 | 21904 | #N/A | #N/A |
| 183_52_O2_19 | DNA repair protein RadC | EKA35833.1 | 22164 | 22661 | #N/A | #N/A |
| 183_52_O2_20 | hypothetical protein PAE2_4137 | EKA51883.1 | 22636 | 23583 | #N/A | #N/A |
| 183_52_O2_21 | Phage-like protein endonuclease-like protein | NP_744643.1 | 23673 | 24677 | #N/A | #N/A |
| 183_52_O2_22 | phage/plasmid-related protein | YP_001186681.1 | 24752 | 25720 | #N/A | #N/A |
| 183_52_O2_23 | hypothetical protein PMI22_03690 | ZP_10699063.1 | 25827 | 26156 | #N/A | #N/A |
| 183_52_O2_24 | hypothetical protein Alide2_0008 | YP_004385964.1 | 26498 | 26983 | #N/A | #N/A |
| 183_52_O2_25 | hypothetical protein Despr_1026 | YP_004194489.1 | 26995 | 27666 | #N/A | #N/A |
| 183_52_O2_26 | hypothetical protein NiasoDRAFT_3049 | ZP_09631892.1 | 28373 | 29053 | #N/A | #N/A |
| 183_52_O2_27 | prevent-host-death family protein | ZP_04934490.1 | 29252 | 29497 | #N/A | #N/A |
| 183_52_O2_28 | hypothetical protein PseS9_11520 | ZP_09710870.1 | 29642 | 29914 | #N/A | #N/A |
| 183_52_O2_29 | phage integrase | YP_006482183.1 | 30192 | 31598 | #N/A | #N/A |
| 183_52_O2_30 | electron transfer flavoprotein subunit alpha | EIK53926.1 | 32163 | 33104 | SEC | Oxido |
| 183_52_O2_31 | electron transfer flavoprotein, beta subunit | EJL08347.1 | 33104 | 33853 | #N/A | Oxido |
| 183_52_O2_32 | enoyl-CoA hydratase/isomerase | BAL24591.1 | 34019 | 34807 | #N/A | #N/A |
| 183_52_O2_33 | phbA2 gene product | YP_004974080.1 | 34838 | 35590 | #N/A | #N/A |
| 183_42_E18_1 | addiction module toxin, RelE/StbE family | ZP_02377280.1 | 364 | 645 | #N/A | #N/A |
| | prot | | | | | |
| 183_42_E18_2 | prevent-host-death family protein | YP_006415110.1 | 635 | 883 | #N/A | #N/A |
| 183_42_E18_3 | conserved hypothetical protein | ZP_04763649.1 | 1120 | 1518 | #N/A | #N/A |
| 183_42_E18_4 | transcriptional regulator, ArsR family | ZP_04762984.1 | 1586 | 1930 | #N/A | #N/A |
| 183_42_E18_5 | hypothetical protein | YP_983052.1 | 2079 | 2288 | #N/A | #N/A |
| 183_42_E18_6 | Rhodanese domain protein | ZP_04763120.1 | 2303 | 2584 | #N/A | #N/A |
| 183_42_E18_7 | OsmC family protein | YP_983056.1 | 2608 | 3054 | #N/A | #N/A |
| 183_42_E18_8 | putative transmembrane protein | ZP_04763118.1 | 3089 | 3280 | #N/A | #N/A |
| 183_42_E18_9 | biotin synthase | ZP_08946418.1 | 3381 | 4460 | #N/A | #N/A |
| 183_42_E18_10 | universal stress protein | YP_157145.1 | 4924 | 5808 | #N/A | #N/A |
| 183_42_E18_11 | lactoylglutathione lyase | ZP_04763114.1 | 5829 | 6305 | #N/A | #N/A |
| 183_42_E18_12 | carbamoyl-phosphate synthase 1 chain ATP- | ZP_09329248.1 | 6434 | 8482 | #N/A | #N/A |
| | bind | | | | | |
| 183_42_E18_13 | carboxyl transferase | ZP_04763112.1 | 8504 | 10036 | #N/A | #N/A |
| 183_42_E18_14 | LAO/AO transport system ATPase | ZP_04763111.1 | 10079 | 11107 | #N/A | #N/A |
| 183_42_E18_15 | methylmalonyl-CoA mutase | ZP_08946425.1 | 11104 | 13272 | #N/A | #N/A |
| 183_42_E18_16 | GntR family transcriptional regulator | ZP_08946426.1 | 13394 | 14029 | #N/A | #N/A |
| 183_42_E18_17 | Ferric reductase domain protein | ZP_04763108.1 | 14033 | 15367 | TM | Oxido |
| | transmembrane | | | | | |
| 183_42_E18_18 | AMP-dependent synthetase and ligase | ZP_08946428.1 | 15582 | 17420 | #N/A | #N/A |
| 183_42_E18_19 | Protein of unknown function (DUF3334) | ZP_10390557.1 | 17539 | 18234 | #N/A | #N/A |
| 183_42_E18_20 | saccharopine dehydrogenase | ZP_08946429.1 | 18235 | 19356 | #N/A | #N/A |
| 183_42_E18_21 | AsnC family transcriptional regulator | ZP_09329239.1 | 19498 | 19950 | #N/A | #N/A |
| 183_42_E18_22 | Endonuclease/exonuclease/phosphatase | ZP_09331304.1 | 20036 | 21010 | #N/A | #N/A |
| 183_42_E18_23 | hypothetical protein IMCC1989_1692 | ZP_08330724.1 | 21061 | 21492 | #N/A | #N/A |
| 183_42_E18_24 | phospholipid/glycerol acyltransferase | ZP_09331305.1 | 22174 | 22764 | #N/A | #N/A |
| 183_42_E18_25 | outer membrane protein/peptidoglycan- | ZP_10389736.1 | 22971 | 23486 | #N/A | #N/A |
| | associat | | | | | |
| 183_42_E18_26 | ChaC family protein | YP_004234936.1 | 23544 | 24134 | #N/A | #N/A |
| 183_42_E18_27 | pyridoxamine 5′-phosphate oxidase | ZP_04762481.1 | 24156 | 24812 | #N/A | #N/A |
| 183_42_E18_28 | hypothetical protein AcdelDRAFT_1713 | ZP_04762482.1 | 24822 | 25088 | #N/A | #N/A |
| 183_42_E18_29 | auxin efflux carrier | ZP_08946084.1 | 25093 | 26049 | #N/A | #N/A |
| 183_42_E18_30 | chromate ion transporter | YP_133847.1 | 26152 | 27405 | #N/A | #N/A |
| 183_42_E18_31 | GMP synthase, large subunit | ZP_04762299.1 | 27490 | 29130 | #N/A | #N/A |
| 183_42_E18_32 | inosine-5′-monophosphate dehydrogenase | ZP_09328399.1 | 29208 | 30683 | #N/A | #N/A |
| 183_42_E18_33 | hypothetical protein KYG_06529 | ZP_09328398.1 | 30760 | 31272 | #N/A | #N/A |
| 183_42_E18_34 | hypothetical protein PMI14_04152 | ZP_10391458.1 | 31295 | 31633 | #N/A | #N/A |
| 183_42_E18_35 | cyclase/dehydrase | ZP_09328396.1 | 31626 | 32066 | #N/A | #N/A |
| 183_42_E18_36 | SsrA-binding protein | ZP_03542969.1 | 32201 | 32674 | #N/A | #N/A |
| 183_42_E18_37 | secreted repeat protein | YP_004126579.1 | 32775 | 33140 | #N/A | #N/A |
| 183_42_E18_38 | RNA polymerase subunit sigma-24 | YP_002552721.1 | 33160 | 33666 | #N/A | #N/A |
| 182_10_L09_1 | LemA family protein | ZP_10200892.1 | 3 | 236 | #N/A | #N/A |
| 182_10_L09_2 | Heat shock protein HtpX | ZP_08647540.1 | 334 | 2166 | #N/A | #N/A |
| 182_10_L09_3 | hypothetical protein Tmz1t_2019 | YP_002355665.1 | 2373 | 2813 | #N/A | #N/A |
| 182_10_L09_4 | Putative alpha/beta-Hydrolase | YP_002354168.1 | 3060 | 3941 | #N/A | #N/A |
| 182_10_L09_5 | major facilitator transporter | YP_284361.1 | 4029 | 5195 | #N/A | #N/A |
| 182_10_L09_6 | excinuclease ABC subunit C | YP_002355941.1 | 5297 | 7114 | #N/A | #N/A |
| 182_10_L09_7 | beta-hexosaminidase | YP_002355942.1 | 7315 | 8460 | #N/A | #N/A |
| 182_10_L09_8 | holo-acyl-carrier-protein synthase | YP_002355943.1 | 8457 | 8837 | #N/A | #N/A |
| 182_10_L09_9 | pyridoxine 5′-phosphate synthase | YP_002355944.1 | 8866 | 9621 | #N/A | #N/A |
| 182_10_L09_10 | DNA repair protein RecO | YP_002355945.1 | 9621 | 10373 | #N/A | #N/A |
| 182_10_L09_11 | GTP-binding protein Era | YP_002355946.1 | 10389 | 11321 | #N/A | #N/A |
| 182_10_L09_12 | ribonuclease III | YP_002355947.1 | 11318 | 11989 | #N/A | #N/A |
| 182_10_L09_13 | hypothetical protein Tmz1t_2313 | YP_002355948.1 | 11994 | 12350 | #N/A | #N/A |
| 182_10_L09_14 | lepB gene product | YP_933144.1 | 12423 | 13211 | #N/A | #N/A |
| 182_10_L09_15 | GTP-binding protein LepA | YP_002355950.1 | 13260 | 15056 | #N/A | #N/A |
| 182_10_L09_16 | glutaredoxin | YP_002355951.1 | 15124 | 15399 | #N/A | #N/A |
| 182_10_L09_17 | protease Do | YP_002355952.1 | 15396 | 16847 | #N/A | #N/A |
| 182_10_L09_18 | positive regulator of sigma E, RseC/MucC | YP_002355953.1 | 16844 | 17314 | #N/A | #N/A |
| 182_10_L09_19 | sigma E regulatory protein, MucB/RseB | YP_002355954.1 | 17311 | 18282 | #N/A | #N/A |
| 182_10_L09_20 | anti sigma-E protein, RseA | YP_002355955.1 | 18279 | 18824 | #N/A | #N/A |
| 182_10_L09_21 | algU gene product | YP_933134.1 | 18834 | 19433 | #N/A | #N/A |
| 182_10_L09_22 | L-aspartate oxidase | YP_002355957.1 | 19622 | 21262 | #N/A | HPG |
| 182_10_L09_23 | hypothetical protein | YP_933132.1 | 21343 | 21852 | #N/A | #N/A |
| 182_10_L09_24 | fabF1 gene product | YP_933131.1 | 21880 | 23115 | TAT | Secretion |
| 182_10_L09_25 | acyl carrier protein | YP_002355960.1 | 23217 | 23456 | #N/A | #N/A |
| 182_10_L09_26 | 3-ketoacyl-(acyl-carrier-protein) reductase | YP_002355961.1 | 23548 | 24297 | #N/A | #N/A |
| 182_10_L09_27 | malonyl CoA-acyl carrier protein | YP_002355962.1 | 24301 | 25230 | #N/A | #N/A |
| | transacylas | | | | | |
| 182_10_L09_28 | 3-oxoacyl-(acyl carrier protein) synthase II | YP_002355963.1 | 25267 | 26232 | #N/A | #N/A |
| 182_10_L09_29 | glycerol-3-phosphate acyltransferase PlsX | YP_002889409.1 | 26229 | 27245 | #N/A | #N/A |
| 182_10_L09_30 | rpmF gene product | YP_933125.1 | 27339 | 27518 | #N/A | #N/A |
| 182_10_L09_31 | metal-binding protein | YP_002889411.1 | 27548 | 28072 | #N/A | #N/A |
| 182_10_L09_32 | maf protein | YP_002889412.1 | 28253 | 28828 | #N/A | #N/A |
| 182_10_L09_33 | uroporphyrin-III C/tetrapyrrole methyltransf | YP_002889413.1 | 28825 | 29574 | SEC | Oxido |
| 182_10_L09_34 | HAD-superfamily hydrolase | YP_002889414.1 | 29656 | 30312 | #N/A | #N/A |
| 182_07_C02_1 | hypothetical protein ebB27 | YP_157502.1 | 3 | 281 | #N/A | #N/A |
| 182_07_C02_2 | hypothetical protein NE1441 | NP_841482.1 | 278 | 457 | #N/A | #N/A |
| 182_07_C02_3 | hypothetical protein ebA893 | YP_157504.1 | 483 | 2396 | #N/A | #N/A |
| 182_07_C02_4 | cysteine synthase B | YP_002890006.1 | 2545 | 3435 | #N/A | #N/A |
| 182_07_C02_5 | tetratricopeptide repeat protein | YP_002890007.1 | 3493 | 4668 | #N/A | #N/A |
| 182_07_C02_6 | hypothetical protein Tmz1t_3033 | YP_002890008.1 | 4674 | 4982 | #N/A | #N/A |
| 182_07_C02_7 | integration host factor subunit beta | YP_002890009.1 | 5070 | 5357 | #N/A | #N/A |
| 182_07_C02_8 | 30S ribosomal protein S1 | YP_002890010.1 | 5369 | 7072 | #N/A | #N/A |
| 182_07_C02_9 | bifunctional 3-phosphoshikimate 1- | YP_002890011.1 | 7161 | 9113 | #N/A | #N/A |
| | carboxyvin | | | | | |
| 182_07_C02_10 | prephenate dehydrogenase | YP_002890012.1 | 9205 | 10092 | #N/A | #N/A |
| 182_07_C02_11 | histidinol-phosphate aminotransferase | YP_002890013.1 | 10106 | 11203 | #N/A | #N/A |
| 182_07_C02_12 | chorismate mutase | YP_002890014.1 | 11382 | 12449 | #N/A | #N/A |
| 182_07_C02_13 | phosphoserine aminotransferase | YP_002890015.1 | 12520 | 13617 | #N/A | #N/A |
| 182_07_C02_14 | DNA gyrase subunit A | YP_002890016.1 | 13617 | 16283 | #N/A | #N/A |
| 182_07_C02_15 | heat shock protein GrpE | YP_002890017.1 | 16421 | 17023 | #N/A | #N/A |
| 182_07_C02_16 | molecular chaperone DnaK | YP_002890018.1 | 17174 | 19090 | #N/A | #N/A |
| 182_07_C02_17 | chaperone protein DnaJ | YP_002890019.1 | 19187 | 20311 | #N/A | #N/A |
| 182_07_C02_18 | hypothetical protein AZL_009250 | YP_003448107.1 | 20468 | 20959 | #N/A | #N/A |
| 182_07_C02_19 | cysteinyl-tRNA synthetase | YP_002890021.1 | 21014 | 22399 | #N/A | #N/A |
| 182_07_C02_20 | cyclophilin type peptidyl-prolyl cis-trans i | YP_002890022.1 | 22638 | 23228 | #N/A | #N/A |
| 182_07_C02_21 | cyclophilin type peptidyl-prolyl cis-trans i | YP_002890023.1 | 23271 | 23765 | #N/A | #N/A |
| 182_07_C02_22 | lpxH gene product | YP_932559.1 | 23812 | 24579 | #N/A | #N/A |
| 182_07_C02_23 | hypothetical protein Tmz1t_0120 | YP_002353815.1 | 24742 | 25392 | #N/A | #N/A |
| 182_07_C02_24 | purC gene product | YP_934376.1 | 25550 | 26485 | #N/A | #N/A |
| 182_07_C02_25 | sugar phosphate permease | YP_005029621.1 | 26559 | 27821 | #N/A | #N/A |
| 182_07_C02_26 | hypothetical protein Tmz1t_1482 | YP_002355137.1 | 28000 | 28773 | #N/A | #N/A |
| 182_07_C02_27 | oligopeptidase A | YP_002355136.1 | 28935 | 31043 | #N/A | #N/A |
| 182_07_C02_28 | PAS/PAC sensor-containing diguanylate | YP_158656.1 | 31065 | 33251 | #N/A | PAS |
| | cycl | | | | | |
| 182_07_C02_29 | methyl-accepting chemotaxis sensory | YP_002354091.1 | 33248 | 33631 | #N/A | MACP |
| | transduc | | | | | |
| 182_13_A07_1 | MACP | EHY78511.1 | 268 | 2289 | TM | MACP |
| 182_13_A07_2 | hypothetical protein A458_15285 | YP_006458707.1 | 2407 | 3087 | #N/A | #N/A |
| 182_13_A07_3 | hypothetical protein A458_15280 | YP_006458706.1 | 3100 | 3831 | #N/A | #N/A |
| 182_13_A07_4 | hypothetical protein A458_15275 | YP_006458705.1 | 3890 | 4246 | #N/A | #N/A |
| 182_13_A07_5 | sodium/sulfate symporter family protein | YP_004715329.1 | 4461 | 6308 | #N/A | #N/A |
| 182_13_A07_6 | alpha/beta hydrolase | EHY78513.1 | 6537 | 7493 | #N/A | #N/A |
| 182_13_A07_7 | transcription elongation factor | YP_006458702.1 | 7490 | 7966 | #N/A | #N/A |
| 182_13_A07_8 | DNA topoisomerase III | YP_006458701.1 | 8071 | 10011 | #N/A | #N/A |
| 182_13_A07_9 | hypothetical protein A458_15250 | YP_006458700.1 | 10200 | 10412 | #N/A | #N/A |
| 182_13_A07_10 | proton-glutamate symporter | YP_004715322.1 | 10540 | 11769 | #N/A | #N/A |
| 182_13_A07_11 | hypothetical protein A458_15240 | YP_006458698.1 | 11964 | 12287 | #N/A | #N/A |
| 182_13_A07_12 | hypothetical protein PstZobell_07400 | EHY77247.1 | 12359 | 13084 | #N/A | #N/A |
| 182_13_A07_13 | ABC transporter permease | YP_001173397.1 | 13610 | 14536 | #N/A | #N/A |
| 182_13_A07_14 | ABC transporter permease | YP_005939690.1 | 14602 | 15708 | #N/A | #N/A |
| 182_13_A07_15 | ABC transporter ATP-binding protein | YP_005939689.1 | 15721 | 17277 | #N/A | #N/A |
| 182_13_A07_16 | transcriptional regulator | EHY77252.1 | 17529 | 18449 | #N/A | #N/A |
| 182_13_A07_17 | gamma-carboxymuconolactone | YP_006458691.1 | 18451 | 18906 | #N/A | #N/A |
| | decarboxylase | | | | | |
| 182_13_A07_18 | short-chain dehydrogenase | EHY76094.1 | 18917 | 19654 | #N/A | #N/A |
| 182_13_A07_19 | tRNA-specific adenosine deaminase | YP_001173390.1 | 19777 | 20247 | #N/A | #N/A |
| 182_13_A07_20 | ABC transporter permease | EHY76096.1 | 20284 | 21078 | #N/A | #N/A |
| 182_13_A07_21 | ABC transporter ATP-binding protein | EHY76097.1 | 21065 | 21868 | #N/A | #N/A |
| 182_13_A07_22 | hypothetical protein | YP_005939681.1 | 21873 | 22850 | #N/A | #N/A |
| 182_13_A07_23 | putative alkyl salicylate esterase | YP_001173386.1 | 22901 | 23650 | #N/A | #N/A |
| 182_13_A07_24 | non-heme iron-dependent enzyme | EHY76100.1 | 23643 | 24623 | #N/A | Oxido |
| 182_13_A07_25 | PAS/PAC sensor hybrid histidine kinase | YP_006458683.1 | 24916 | 27147 | SEC | PAS |
| 182_13_A07_26 | PAS domain S-box | YP_006458682.1 | 27386 | 29071 | #N/A | PAS |
| 182_13_A07_27 | circadian oscillation regulator | EHY79520.1 | 29055 | 30581 | #N/A | #N/A |
| 182_13_A07_28 | putative ABC1 protein | EHY79521.1 | 31019 | 32320 | #N/A | #N/A |
| 182_13_A07_29 | short chain dehydrogenase/reductase family | EHY79522.1 | 32341 | 33042 | #N/A | Oxido |
| | oxidor | | | | | |
| 182_13_A07_30 | PAS domain S-box | YP_005939664.1 | 33015 | 35147 | #N/A | PAS |
| 182_13_A07_31 | gamma-glutamyltransferase | EHY79524.1 | 35567 | 37240 | #N/A | #N/A |
| 182_13_A07_32 | glyoxalase/bleomycin resistance | YP_006458675.1 | 37373 | 37765 | #N/A | HPG |
| | protein/diox | | | | | |
| 182_13_A07_34 | hypothetical protein PST_2282 | YP_001172783.1 | 38308 | 38778 | #N/A | #N/A |
| 182_13_A07_35 | hypothetical protein Pext1s1_03389 | ZP_10435433.1 | 39005 | 39409 | #N/A | #N/A |
| 182_13_A07_36 | LysR family transcriptional regulator | YP_006458671.1 | 39862 | 40545 | #N/A | #N/A |
| 183_29_M04_1 | K+ potassium transporter | ZP_08949167.1 | 1 | 1551 | #N/A | #N/A |
| 183_29_M04_2 | benzoate transporter | ZP_04764009.1 | 1697 | 2887 | SEC | MDES |
| 183_29_M04_3 | glutathione synthetase | YP_983953.1 | 2925 | 3881 | #N/A | #N/A |
| 183_29_M04_4 | integrase catalytic subunit | YP_551887.1 | 9978 | 10829 | #N/A | #N/A |
| 183_29_M04_5 | transposase is3/is911 family protein | YP_004154633.1 | 10883 | 11209 | #N/A | #N/A |
| 183_29_M04_6 | DSBA oxidoreductase, Twin-arginine | YP_984674.1 | 11257 | 11910 | TAT | Oxido |
| | translocation pathway signal | | | | | |
| 183_29_M04_7 | sporulation domain-containing protein | YP_968796.1 | 11965 | 12582 | #N/A | #N/A |
| 183_29_M04_8 | arginyl-tRNA synthetase | ZP_09331017.1 | 12597 | 14294 | #N/A | #N/A |
| 183_29_M04_9 | hypothetical protein Acav_0473 | YP_004232963.1 | 14348 | 14806 | #N/A | #N/A |
| 183_29_M04_10 | transcriptional regulator, LysR family | ZP_04763015.1 | 14803 | 15735 | #N/A | #N/A |
| 183_29_M04_11 | coenzyme A transferase | ZP_08950393.1 | 15857 | 17803 | #N/A | #N/A |
| 183_29_M04_12 | malate synthase G | ZP_09327224.1 | 18008 | 20209 | #N/A | #N/A |
| 183_29_M04_13 | putative monovalent cation/H+ antiporter | ZP_09327225.1 | 20320 | 20661 | #N/A | #N/A |
| | subu | | | | | |
| 183_29_M04_14 | putative monovalent cation/H+ antiporter | ZP_09327226.1 | 20672 | 20947 | #N/A | #N/A |
| | subu | | | | | |
| 183_29_M04_15 | putative K(+)/H(+) antiporter subunit E | ZP_09327227.1 | 20944 | 21519 | #N/A | #N/A |
| 183_29_M04_16 | putative monovalent cation/H+ antiporter | ZP_09327228.1 | 21516 | 23150 | #N/A | #N/A |
| | subu | | | | | |
| 183_29_M04_17 | putative monovalent cation/H+ antiporter | ZP_09327229.1 | 23150 | 23542 | #N/A | #N/A |
| | subu | | | | | |
| 183_29_M04_18 | putative monovalent cation/H+ antiporter | ZP_09327230.1 | 23598 | 26441 | #N/A | #N/A |
| | subu | | | | | |
| 183_29_M04_19 | 4-oxalocrotonate tautomerase | ZP_09327232.1 | 27298 | 27486 | #N/A | #N/A |
| 183_29_M04_20 | emrB/QacA subfamily drug resistance | ZP_09327233.1 | 27661 | 29133 | TM | MDES |
| | transport | | | | | |
| 183_29_M04_21 | class-II glutamine amidotransferase | ZP_09327234.1 | 29155 | 29922 | #N/A | #N/A |
| 183_29_M04_22 | glyoxalase/bleomycin resistance | ZP_09327922.1 | 29949 | 30329 | TM | HPG |
| | protein/dioxy | | | | | |
| 183_29_M04_23 | hypothetical protein KYG_01427 | ZP_09327395.1 | 30741 | 31250 | #N/A | #N/A |
| 183_29_M04_24 | hypothetical protein Rfer_4013 | YP_525242.1 | 32011 | 32952 | #N/A | #N/A |
| 183_29_M04_25 | transposase, IS4 family protein | YP_984420.1 | 33103 | 34191 | #N/A | #N/A |
| 183_29_M04_26 | hypothetical protein Tmz1t_3596 | YP_002890566.1 | 34340 | 34744 | #N/A | #N/A |
| 183_29_M04_27 | hypothetical protein | ACG50166.1 | 34968 | 37064 | #N/A | #N/A |
| 183_29_M04_28 | putative transposon resolvase | YP_003034068.1 | 37080 | 37703 | #N/A | #N/A |
| 183_29_M04_29 | transposase | NP_061389.1 | 37795 | 40803 | #N/A | #N/A |
| 183_29_M04_30 | hypothetical protein Tmz1t_3596 | YP_002890566.1 | 40800 | 41213 | #N/A | #N/A |
| 183_38_D19_1 | parvulin-like peptidyl-prolyl isomerase | ZP_08570556.1 | 3 | 1949 | #N/A | #N/A |
| 183_38_D19_2 | ABC-type antimicrobial peptide transport | ZP_08570001.1 | 2032 | 2817 | #N/A | #N/A |
| | sy st | | | | | |
| 183_38_D19_3 | oligopeptide/dipeptide ABC transporter, | ZP_08570002.1 | 2810 | 3808 | #N/A | #N/A |
| | ATP-b | | | | | |
| 183_38_D19_4 | ABC-type antimicrobial peptide transport | ZP_08570003.1 | 3808 | 4701 | #N/A | #N/A |
| | sy st | | | | | |
| 183_38_D19_5 | ABC-type antimicrobial peptide transport | ZP_08570004.1 | 4703 | 5722 | #N/A | #N/A |
| | sy st | | | | | |
| 183_38_D19_6 | dipeptide transport system substrate-binding | ZP_09988451.1 | 5719 | 7329 | #N/A | #N/A |
| 183_38_D19_7 | psp operon transcriptional activator PspF | ZP_08570006.1 | 7326 | 8423 | #N/A | #N/A |
| 183_38_D19_8 | phage shock protein A | ZP_09988448.1 | 8608 | 9276 | #N/A | #N/A |
| 183_38_D19_9 | phage shock protein B | ZP_09988447.1 | 9302 | 9544 | #N/A | #N/A |
| 183_38_D19_10 | phage shock protein C | ZP_09988446.1 | 9531 | 9941 | #N/A | #N/A |
| 183_38_D19_11 | hypothetical protein AGRI_06402 | ZP_10351220.1 | 9987 | 11363 | #N/A | #N/A |
| 183_38_D19_12 | UPF0283 membrane protein | ZP_09988444.1 | 11360 | 12379 | #N/A | #N/A |
| 183_38_D19_13 | methionine gamma-lyase | ZP_08570012.1 | 12512 | 13717 | #N/A | #N/A |
| 183_38_D19_14 | phenylalanine 4-monooxygenase | ZP_10351216.1 | 13894 | 14685 | #N/A | #N/A |
| 183_38_D19_15 | pterin-4-alpha-carbinolamine dehydratase | ZP_10351215.1 | 14729 | 15070 | #N/A | #N/A |
| 183_38_D19_16 | transcriptional regulator of aroF, aroG, tyrA | ZP_09988440.1 | 15219 | 16781 | #N/A | #N/A |
| 183_38_D19_17 | 4-hydroxyphenylpyruvate dioxygenase | ZP_10493291.1 | 16999 | 18072 | #N/A | #N/A |
| 183_38_D19_18 | homogentisate 1,2-dioxygenase | ZP_08570017.1 | 18065 | 19204 | #N/A | #N/A |
| 183_38_D19_19 | 2-keto-4-pentenoate hydratase/2-oxohepta- | ZP_08570018.1 | 19272 | 20294 | #N/A | #N/A |
| | 3-en | | | | | |
| 183_38_D19_20 | maleylacetoacetate isomerase | ZP_09988436.1 | 20380 | 21018 | #N/A | #N/A |
| 183_38_D19_21 | response regulator | ZP_06039558.1 | 21220 | 22398 | #N/A | #N/A |
| 183_38_D19_23 | lytic murein transglycosylase | ZP_10351206.1 | 22819 | 24411 | #N/A | #N/A |
| 183_38_D19_24 | hydroxyacylglutathione hydrolase | ZP_08570023.1 | 24543 | 25322 | #N/A | #N/A |
| 183_38_D19_25 | SAM-dependent methyltransferase | ZP_08570024.1 | 25385 | 26119 | #N/A | #N/A |
| 183_38_D19_26 | hypothetical protein | YP_942205.1 | 26180 | 26626 | #N/A | #N/A |
| 183_38_D19_27 | acetyltransferase | ZP_08570026.1 | 26610 | 27194 | #N/A | #N/A |
| 183_38_D19_28 | hypothetical protein Rhein_1400 | ZP_08570029.1 | 27506 | 28039 | #N/A | #N/A |
| 183_38_D19_29 | DNA/RNA helicase, superfamily II | ZP_08570030.1 | 28188 | 29513 | #N/A | #N/A |
| 183_38_D19_30 | cold shock protein | ZP_08570031.1 | 29799 | 30011 | #N/A | #N/A |
| 183_38_D19_31 | nucleotidyltransferase/DNA polymerase | ZP_08570032.1 | 30225 | 31280 | #N/A | #N/A |
| | involve | | | | | |
| 183_38_D19_32 | glycine hydroxymethyltransferase | ZP_09990430.1 | 31559 | 32812 | #N/A | #N/A |
| 183_38_D19_33 | transcriptional regulator NrdR | ZP_08570034.1 | 32881 | 33333 | #N/A | #N/A |
| 183_38_D19_34 | riboflavin biosynthesis protein RibD | ZP_08570035.1 | 33337 | 34458 | #N/A | #N/A |
| 183_38_D19_35 | riboflavin synthase | ZP_09990427.1 | 34461 | 35111 | #N/A | #N/A |
| 183_38_D19_36 | 3,4-dihydroxy-2-butanone 4-phosphate | ZP_09990426.1 | 35160 | 36269 | #N/A | #N/A |
| | synthase | | | | | |
| 183_38_D19_37 | 6,7-dimethyl-8-ribityllumazine synthase | ZP_08570038.1 | 36428 | 36892 | #N/A | #N/A |
| 183_38_D19_38 | transcription antitermination factor NusB | ZP_08570039.1 | 36902 | 37315 | #N/A | #N/A |
| 183_38_D19_39 | thiamine-monophosphate kinase | ZP_08570040.1 | 37344 | 38303 | #N/A | #N/A |
| 183_38_D19_40 | phosphatidylglycerophosphatase A | ZP_09757413.1 | 38303 | 38779 | #N/A | #N/A |
| 183_38_D19_41 | diguanylate cyclase (GGDEF) domain- | ZP_08570042.1 | 38807 | 40009 | SEC | PAS |
| | containing | | | | | |
| 182_19_A11_2 | diguanylate cyclase/phosphodiesterase | YP_001186817.1 | 141 | 1979 | #N/A | #N/A |
| 182_19_A11_3 | FKBP-type peptidylprolyl isomerase | EIK51391.1 | 2349 | 3086 | #N/A | #N/A |
| 182_19_A11_4 | recombination associated protein | YP_004379126.1 | 3739 | 4656 | #N/A | #N/A |
| 182_19_A11_5 | flagellar hook protein FlgE | EIK53823.1 | 4756 | 6153 | SEC | Secretion |
| 182_19_A11_6 | flagellar basal body rod modification protei | YP_006523702.1 | 6190 | 6873 | #N/A | Secretion |
| 182_19_A1l_7 | flgC gene product | YP_001188336.1 | 6886 | 7329 | SEC | Secretion |
| 182_19_A11_8 | flagellar basal body rod protein FlgB | YP_004713701.1 | 7332 | 7736 | #N/A | Secretion |
| 182_19_A11_9 | glmS gene product | YP_003899504.1 | 7960 | 9789 | #N/A | #N/A |
| 182_19_A11_10 | UDP-glucose 4-epimerase | ZP_08824132.1 | 9804 | 10820 | #N/A | #N/A |
| 182_19_A11_11 | glutamyl-tRNA synthetase | ZP_10760857.1 | 10845 | 12338 | #N/A | #N/A |
| 182_19_A11_12 | LysR family transcriptional regulator | EJO93868.1 | 12403 | 13317 | #N/A | #N/A |
| 182_19_A11_13 | secretion protein HlyD family protein | ZP_09708733.1 | 13422 | 14453 | TM | Secretion |
| 182_19_A11_14 | EmrB/QacA family drug resistance | YP_001347371.1 | 14446 | 15981 | TAT | MDES |
| | transporter | | | | | |
| 182_19_A11_15 | glycine/D-amino acid oxidase | ZP_10990193.1 | 16187 | 17467 | #N/A | HPG |
| 182_19_A11_16 | hypothetical protein A471_09819 | EJO94022.1 | 17545 | 17946 | #N/A | #N/A |
| 182_19_A11_17 | TPR repeat-containing protein | YP_005428973.1 | 17946 | 18896 | #N/A | #N/A |
| 182_19_A11_18 | nitrite reductase | ZP_08817914.1 | 19113 | 20759 | SEC | Oxido |
| 182_19_A11_19 | cytochrome c551/c552 | YP_005029489.1 | 20832 | 21188 | TAT | Oxido |
| 182_19_A11_20 | tetraheme protein NirT | YP_001174001.1 | 21268 | 21870 | TM | Oxido |
| 182_19_A11_21 | denitrification system component | YP_002889595.1 | 21922 | 22800 | #N/A | #N/A |
| | cytochrome | | | | | |
| 182_19_A11_22 | TPR repeat-containing protein | YP_006532722.1 | 22859 | 24058 | #N/A | #N/A |
| 182_19_A11_23 | tRNA-dihydrouridine synthase A | EJX14582.1 | 24273 | 25292 | #N/A | #N/A |
| 182_19_A11_24 | transaldolase B | EJO95866.1 | 25344 | 26297 | #N/A | #N/A |
| 182_19_A11_25 | anti-sigma-factor antagonist | YP_001268884.1 | 26286 | 26774 | #N/A | #N/A |
| 182_19_A11_26 | response regulator receiver protein | YP_001187423.1 | 26771 | 27961 | #N/A | MACP |
| 182_19_A11_27 | type IV pilus assembly PilZ | ZP_09282631.1 | 28154 | 28459 | #N/A | Secretion |
| 182_19_A11_28 | VacJ family lipoprotein | ZP_09709915.1 | 28456 | 29169 | #N/A | #N/A |
| 182_19_A11_29 | RND family efflux transporter MFP subunit | YP_005090623.1 | 29335 | 30486 | SEC | MDES |
| 182_19_A11_30 | macB gene product | YP_932115.1 | 30490 | 32436 | #N/A | #N/A |
| 182_19_A11_31 | RND efflux system, outer membrane | YP_691970.1 | 32426 | 32797 | #N/A | #N/A |
| 182_06_L14_1 | hypothetical protein PSJM300_10595 | YP_006524541.1 | 2 | 220 | #N/A | #N/A |
| 182_06_L14_2 | hypothetical protein PstZobell_17634 | EHY79247.1 | 234 | 911 | #N/A | #N/A |
| 182_06_L14_3 | antirestriction protein family protein | YP_004380665.1 | 1053 | 1565 | #N/A | #N/A |
| 182_06_L14_4 | hypothetical protein PST_0625 | YP_001171173.1 | 1863 | 3059 | #N/A | #N/A |
| 182_06_L14_5 | XRE family transcriptional regulator | YP_001171172.1 | 3059 | 3310 | #N/A | #N/A |
| 182_06_L14_6 | hypothetical protein PSJM300_10590 | YP_006524540.1 | 3685 | 3933 | #N/A | #N/A |
| 182_06_L14_7 | hypothetical protein PSJM300_10585 | YP_006524539.1 | 3934 | 4416 | #N/A | #N/A |
| 182_06_L14_8 | ifsy-2 prophage protein | ZP_10670406.1 | 4510 | 5193 | #N/A | #N/A |
| 182_06_L14_9 | error-prone DNA polymerase | EHY77418.1 | 5280 | 8369 | #N/A | #N/A |
| 182_06_L14_10 | DNA-specific endonuclease I | ZP_04934525.1 | 8840 | 9529 | #N/A | #N/A |
| 182_06_L14_11 | PAS/PAC sensor hybrid histidine kinase | YP_005940175.1 | 9671 | 11902 | SEC | PAS |
| 182_06_L14_12 | hypothetical protein CF510_08712 | EIE46546.1 | 12132 | 12932 | #N/A | #N/A |
| 182_06_L14_14 | hypothetical protein | YP_002800206.1 | 13469 | 13801 | #N/A | #N/A |
| 182_06_L14_16 | hypothetical protein Aasi_0901 | YP_001957997.1 | 14375 | 15694 | #N/A | #N/A |
| 182_06_L14_17 | hypothetical protein HMPREF9551_05665 | ZP_07192991.1 | 15676 | 16353 | #N/A | #N/A |
| 182_06_L14_18 | ABC-type transporter, ATPase and | YP_006524532.1 | 16350 | 18029 | #N/A | #N/A |
| | permease co | | | | | |
| 182_06_L14_19 | Zn-dependent hydrolase | YP_006523933.1 | 18139 | 19035 | SEC | Oxido |
| 182_06_L14_20 | AraC family transcriptional regulator | ZP_09282862.1 | 19160 | 20149 | #N/A | #N/A |
| 182_06_L14_21 | isochorismatase hydrolase | YP_006523935.1 | 20256 | 20804 | #N/A | #N/A |
| 182_06_L14_22 | AraC family transcriptional regulator | YP_006523936.1 | 21288 | 22250 | #N/A | #N/A |
| 182_06_L14_23 | 3-demethylubiquinone-9 3- | YP_006523925.1 | 22438 | 22917 | #N/A | #N/A |
| | methyltransferase | | | | | |
| 182_06_L14_24 | amino-acid ABC transporter ATP-binding | YP_006523926.1 | 22998 | 23756 | #N/A | #N/A |
| | prote | | | | | |
| 182_06_L14_25 | cystine ABC transporter, permease protein, | YP_005939005.1 | 23756 | 24424 | #N/A | #N/A |
| | P | | | | | |
| 182_06_L14_26 | cystine transporter subunit | YP_001172847.1 | 24421 | 25215 | #N/A | #N/A |
| 182_06_L14_27 | D-cysteine desulfhydrase | YP_006523929.1 | 25320 | 26324 | #N/A | #N/A |
| 182_06_L14_28 | transcriptional activator | YP_455420.1 | 26423 | 27145 | #N/A | #N/A |
| 182_06_L14_29 | hypothetical protein PSJM300_10525 | YP_006524529.1 | 27333 | 27599 | #N/A | #N/A |
| 182_06_L14_30 | hypothetical protein Nwat_3173 | YP_003762209.1 | 28295 | 29581 | #N/A | #N/A |
| 182_06_L14_31 | conserved hypothetical protein | ZP_08371866.1 | 29600 | 31267 | #N/A | #N/A |
| 182_06_L14_32 | putative chromosome segregation ATPase | CAJ87561.1 | 31264 | 33141 | #N/A | #N/A |
| 182_06_L14_33 | hypothetical protein eco1045 | CAJ87560.1 | 33151 | 33663 | #N/A | #N/A |

Oxido = Oxidoreductase activity; Secretion = Secretion apparatus or signal; MACP = Methyl-accepting chemotaxis protein; PAS = PAS domain containing sensor; HPG = Hydrogen peroxide generating; and MDES = Multidrug efflux superfamily
SEC = Sec signal peptide predicted; TM = TM segment predicted; and TAT = Tat signal peptide predicted

GC-MS profiles of transposon mutants are shown in FIG. 12, where the chromatogram compares two transposon mutants (i.e. position 4949 and position 55060) identified by screening with the PemrR-GFP biosensor, wherein both are known to be interrupting putative oxidoreductase open reading frames. The data was normalized to an empty fosmid clone (i.e. 182_08_C21). Lignin related compounds 2,4-dihydroxybenzoic acid, 1,4-dihydroxy-2,6-dimethoxybenzene and benzoic acid are marked by A, B and C. There are clear differences shown between the two transposon mutants and the empty fosmid clone.

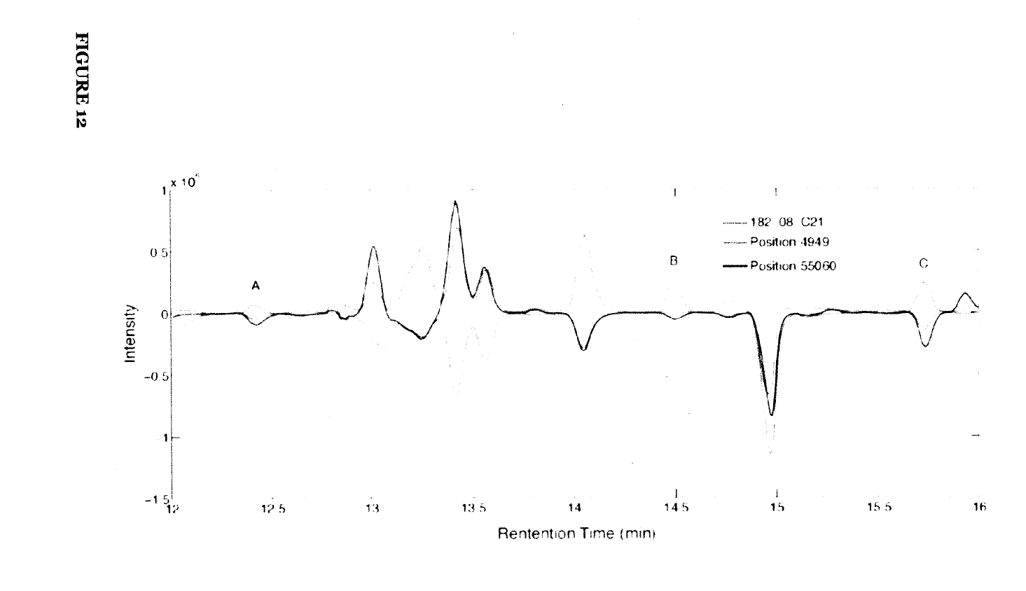

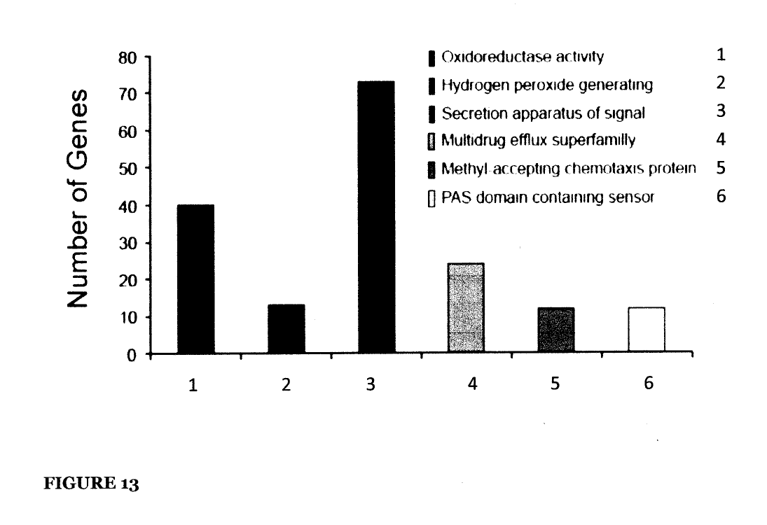

FIG. 13 provides a graphical representation of the relative proportions of genes grouped into six functional classes, implicated in lignin transformation phenotypes (out of 813 total genes) in the active fosmids identified in the exemplary screen. It is interesting to note that these 6 functional classes are consistently represented in the isolated fosmids and with the exception of the secretion apparatus and perhaps the oxidoreductase, these genes are represented quite consistently within the active fosmids identified in this exemplary screen.

### Example 5: Identification and Testing of MIE p10c20

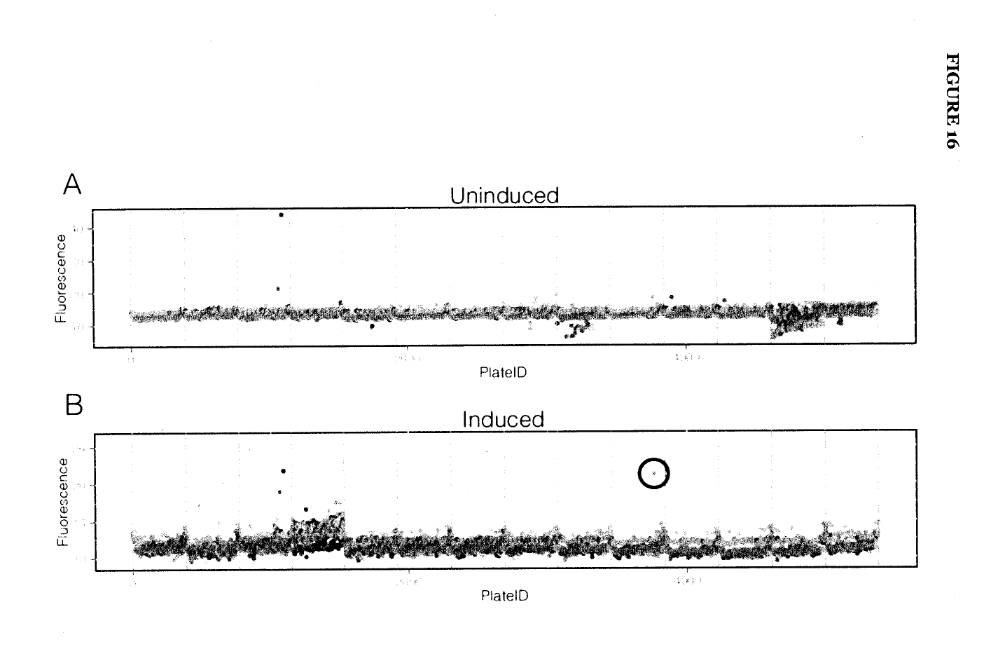

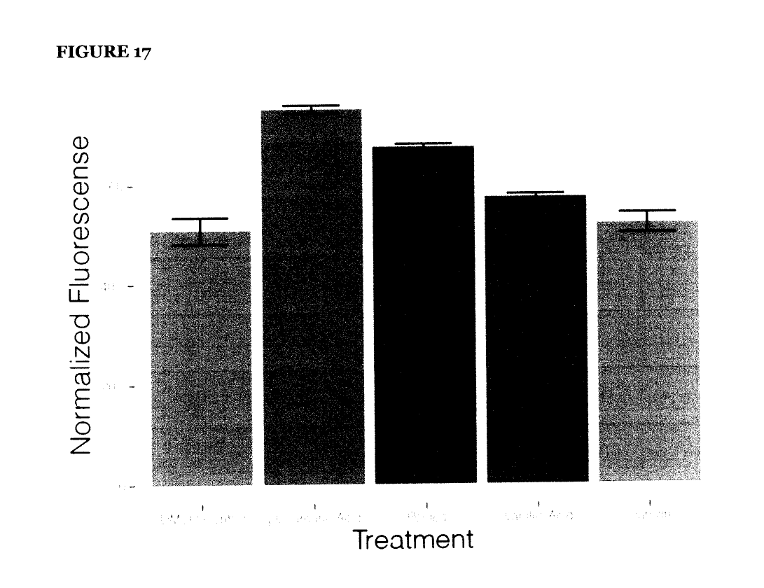

A metagenomic DNA library was “retroffited” as described herein and shown in FIG. 15 to identify a metabolite induced element (MIE). In FIG. 16 fluoresence plots are shown for a retroffited metagenomic library assayed for fluorescence emitted by the fluorescent marker. FIG. 16 (A) shows the fluorescence emitted by an uninduced (i.e. no metabolite of interest is added) library and (B) shows the fluorescence emitted by an induced (i.e. where the metabolite of interest is added) library. Induction was by a pool of pCoumaric acid, Vanillic acid and Vanillin. The circled data point that represents a fosmid clone harboring a candidate MIE (p10c20) which was selected for further investigation. In FIG. 17 a bar graph is shown of an assay to validate the responsiveness of the fosmid clone identified in FIG. 16 (p10c20), wherein the MIE p10c20 was found to be most responsive to 1 mM pCoumaric acid, which makes the reporter system encoded in p10c20 potentially useful to detect heterologous metabolite secretion of chemical transformation resulting in the production of pCoumaric acid.

Although various embodiments of the invention are disclosed herein, many adaptations and modifications may be made within the scope of the invention in accordance with the common general knowledge of those skilled in this art. Such modifications include the substitution of known equivalents for any aspect of the invention in order to achieve the same result in substantially the same way. Numeric ranges are inclusive of the numbers defining the range. The word “comprising” is used herein as an open-ended term, substantially equivalent to the phrase “including, but not limited to”, and the word “comprises” has a corresponding meaning. As used herein, the singular forms “a”, “an” and “the” include plural referents unless the context clearly dictates otherwise. Thus, for example, reference to “a thing” includes more than one such thing. Citation of references herein is not an admission that such references are prior art to an embodiment of the present invention. Any priority document(s) and all publications, including but not limited to patents and patent applications, cited in this specification are incorporated herein by reference as if each individual publication were specifically and individually indicated to be incorporated by reference herein and as though fully set forth herein. The invention includes all embodiments and variations substantially as hereinbefore described and with reference to the examples and drawings.

### REFERENCES

1. Okuta et al. Gene (1998) 212:221-228.
2. Henne et al. Appl. Environ. Microbiol. (1999) 65:3901-3907.
3. Uchiyama et al. Nature Biotechnology (2005) 23(1):88-93.
4. Uchiyama and Miyazaki Appl. Environ. Microbiol. (2010) 76(21):7029-7035.
5. Uchiyama and Miyazaki PLOS ONE (2013) 8(9):e75795.
6. Zakzeski, J. et al. The Catalytic Valorization of Lignin for the Production of Renewable Chemicals. Chem. Rev. 110, 3552-3599 (2010).
7. Ruiz-Duenas, F. J & Martinez, A. T. Microbial degradation of lignin: how a bulky recalcitrant polymer is efficiently recycled in nature and how we can take advantage of this. Micr Biotechnol. 2, 164-177 (2009).
8. Boerjan, W., Ralph, J. & Baucher, M. Lignin Biosynthesis. Annu. Rev. Plant Biol. 54, 519-546 (2003).
9. Floudas, D. et al. The Paleozoic Origin of Enzymatic Lignin Decomposition Reconstructed from 31 Fungal Genomes. Science 336, 1715-1719 (2012).
10. Singh, R. et al. Improved manganese-oxidizing activity of DypB, a peroxidase from a lignolytic bacterium. ACS Chem. Biol. 8, 700-706 (2013).
11. Khudayakoc, J. I. et al. Global transcriptome response to ionic liquid by a tropical rain forest soil bacterium, Enterobacter lignolyticus. PNAS. doi:10.1073/pnas.1112750109 (2012).
12. Brown, M. E., Barros, T. & Chang, C. Y. Identification and characterization of a multifunctional dye peroxidase from a lignin reactive bacterium. ACS Chem Bio. 7, 2074-2081 (2012).
13. Bugg, T. D. H. et al. Pathways for degradation of lignin in bacteria and fungi. Nat. Prod. Rep. 28, 1883-1896 (2011).
14. Rodgers, C. et al. Designer laccases: a vogue for high-potential fungal enzymes? Trends Biotechnol. 2, 63-72 (2009).
15. Ahmad, M. et al. Development of novel assays for lignin degradation: comparative analyses of bacterial and fungal lignin degraders. Mol. BioSyst. 6, 815-821 (2010).
16. Zaslaver, A. et al. A comprehensive library of fluorescent transcriptional reporters for Escherichia coli. Nat. Meth. 3, 623-628 (2006).
17. Brooun, A., Tomashek, J. J. & Lewis, K. Purification and Ligand Binding of EmrR, a Regulator of a Multridrug Transporter. J. Bacteriol. 181, 5131-5133 (1999).
18. Xiong, A. et al. The EmrR Protein Represses the Escherichia coli emrRAB Multidrug Resistance operon by directly binding to its promoter region. Antimicrob. Agents Chemother. 44, 2905-2907 (2000).
19. Strapoc, D. et al. Methane-Producing Microbial Community in a Coal Bed of the Illinois Basin. Appl. Environ. Microbiol. 74, 2424-2432 (2008).
20. An, D. et al. Metagenomics of hydrocarbon resources environments indicates aerobic taxa and genes to be unexpectedly common. Environ. Sci. Technol. DOI: 10.1021/es4020184 (2013).
21. Martinez, A., Bradley, A. S., Waldbauer, J. R., Summons, R. E. & Delong, E. F. Proteorhodopsin photosystem gene expression enables photophosphorylation in a heterologous host. PNAS 104, 5590-5595 (2007).
22. Arato, C., Pye, E. K. and Gjennestad, G. The lignol approach to biorefining of woody biomass to produce ethanol and chemicals. Appl Biochem Biotech. 123, 871-882 (2005).
23. Arfi, Y. et al. Characterization of salt-adapted secreted lignocellulolytic enzymes from the mangrove fungus Pestalotiopsis sp. Nat. Commun. doi: 10.1038/ncomms2850 (2013).
24. Yakovlev, I. A. et al. Genes associated with lignin degradation in the polyphagous white-rot pathogen Heterobasidion irregular show substrate-specific regulation. Fungal Genet. Biol. 56, 17-24 (2013).
25. He, S. et al. Comparative metagenomic and metatransciptomic analysis of hindgut paunch microbiota in wood- and dung feeding higher termites. PLoS ONE. 8(4): e61126 (2012).
26. Shapiro, B. J. et al. Population genomic of early events in ecological differentiation of bacteria. Science. 336, 48-51 (2012).
27. Polz, M. F., Alm, E. J. & Hanage, W. P. Horizontal gene transfer and the evolution of bacterial and archaeal population structure. Trends Genet. 29, 170-175 (2013).
28. Oliver, K. M., Degman, P. H., Hunter, M. S. & Moran, N. A. Bacteriophages encode factors required for protection in symbiotic mutualism. Science. 325, 992-994 (2009).
29. Moran, N. A., Degman, P. H., Santos, S. R., Dunbar, H. E. & Ochman, H. The players in mutualistic symbiosis: insects, bacteria, viruses and virulence genes. PNAS. 102, 16919-16926 (2005).
30. Lee, S. & Hallam, S. J. Extraction of high molecular weight DNA from soils and sediments. J. Vis. Exp. (33), e1569, doi:10.3791/1569 (2009).

1-44. (canceled)

45. A method comprising:
(a) randomly inserting a mobile genetic element into a first library to produce a randomly inserted first library, wherein the mobile genetic element comprises a promoter-less reporter gene;
(b) screening the randomly inserted first library by adding a metabolite of interest;
(c) detecting reporter gene expression following the addition of the metabolite of interest to identify a metabolite induced element (MIE);
(d) preparing a reporter strain, the reporter strain comprising:
(i) the MIE; and
(ii) a reporter gene adjacent the MIE;
(e) co-culturing heterologous host cells expressing a second library with the reporter strain; and
(f) detecting the reporter gene activity in the co-culture.

46. The method of claim 45, further comprising testing the MIE for specificity and sensitivity to the metabolite of interest prior to co-culturing the heterologous host cells expressing a functional library with the reporter strain.

47. The method of claim 46, further comprising engineering the MIE to obtain the desired substrate specificity and sensitivity following testing the MIE for specificity and sensitivity to the metabolite of interest.

48. The method of claim 46, wherein the functional library is a fosmid library.

49. The method of claim 45, wherein the second library is a functional library, and further comprising mutagenesis of the functional library host cells, producing reporter strain activity, and further screening for production of the metabolite of interest.

50. The method of claim 45, wherein the first library is a functional library, and wherein the MIE is obtained from the functional library.

51. The method of claim 45, wherein the reporter strain is a bacterial cell.

52. The method of claim 45, wherein the heterologous host cells expressing a functional library are bacterial cells.

53. The method of claim 45, further comprising isolating the co-culture having reporter gene activity.

54. The method of claim 53, further comprising culturing the host cells having reporter gene activity to produce the metabolite of interest.

55. A method comprising:
(a) randomly inserting a mobile genetic element into a first library to produce a randomly inserted first library, wherein the mobile genetic element comprises a promoter-less reporter gene;
(b) screening the randomly inserted first library by adding a metabolite of interest;
(c) detecting reporter gene expression following the addition of the metabolite of interest to identify a metabolite induced element (MIE); and
(d) preparing a reporter strain, the reporter strain comprising:
(i) the MIE; and
(ii) a reporter gene adjacent the MIE.

56. The method of claim 55, further comprising the step of:
(e) co-culturing heterologous host cells expressing a second library with the reporter strain.

57. The method of claim 56, further comprising the step of:
(f) detecting the reporter gene activity in the co-culture.

58. The method of claim 55, further comprising testing the MIE for specificity to the metabolite of interest prior to co-culturing the heterologous host cells expressing a functional metagenomic library with the reporter strain.

59. The method of claim 58, further comprising engineering the MIE to obtain the desired substrate specificity following testing the MIE for specificity to the metabolite of interest.

60. The method of claim 55, wherein the functional library is a fosmid library.

61. The method of claim 55, further comprising mutagenesis of functional library host cells producing reporter strain activity and further screening for production of the metabolite of interest.

62. The method of claim 55, wherein the reporter strain is a bacterial cell.

63. The method of claim 55, further comprising testing the MIE for sensitivity to the metabolite of interest prior to co-culturing the heterologous host cells expressing a functional metagenomic library with the reporter strain.

64. The method of claim 45, further comprising engineering the MIE to obtain the desired substrate sensitivity following testing the MIE for sensitivity to the metabolite of interest.


SEQUENCE LISTING (5 sequences)

SEQ ID NO: 1 - DNA, 25 bp, Artificial Sequence
  primer
gcggaattccgcagcattatcatcc

SEQ ID NO: 2 - DNA, 30 bp, Artificial Sequence
  primer
gcgaagcttcctgcaggtctggacatttat

SEQ ID NO: 3 - DNA, 31 bp, Artificial Sequence
  primer
gcggaattcatggatagttcgtttacgccca

SEQ ID NO: 4 - DNA, 31 bp, Artificial Sequence
  primer
gcgaagcttttagctcatcgcttcgagaacc

SEQ ID NO: 5 - DNA, 235 bp, Escherichia coli
cgcagcattatcatcccaacactgcttagtgcgctggcctatgggctcgcctggaaagtgatggcgattatataacccacaagaatcatttttctaaaacaatacatttactttatttgtcactgtcgttactatatcggctgaaattaatgaggtcatacccaaatggatagttcgtttacgcccattgaacaaatgctaaaatttcgcgccagccgccacgaagattttcctt